(19) United States
(12) Patent Application Publication
(10) Pub. No.: US 2025/0195431 A1
(43) Pub. Date: Jun. 19, 2025

(54) LIPID NANOPARTICLES FOR TARGETED DELIVERY OF MRNA

(71) Applicant: Trustees of Tufts College, Medford, MA (US)

(72) Inventors: Qiaobing Xu, Lexington, MA (US); Changfeng Huang, Arlington, MA (US)

(21) Appl. No.: 18/845,749

(22) PCT Filed: Mar. 13, 2023
(86) PCT No.: PCT/US23/15119

§ 371 Date: Sep. 10, 2024

Related U.S. Application Data
(60) Provisional application No. 63/318,971, filed on Mar. 11, 2022.

(51) Int. Cl. A61K 9/1272; A61K 9/51; A61K 31/7105; A61K 38/45; A61K 39/00; A61K 39/215; A61K 48/00; A61P 3/00; A61P 9/10; A61P 31/14; C12N 7/00; C12N 9/80; C12N 15/11

(52) U.S. Cl. CPC A61K 9/1272; A61K 9/5123; A61K 31/7105; A61K 38/45; A61K 39/215; A61K 48/005; A61P 3/00; A61P 9/10; A61P 31/14; C12N 7/00; C12N 9/80; C12N 15/111; C12Y 204/01017; A61K 2039/53; A61K 2039/55555; C12N 2310/20; C12N 2770/20034; C12Y 305/04004

(57) ABSTRACT

Disclosed are compositions including a pharmaceutical agent and a lipid composition; wherein the pharmaceutical agent is assembled with the lipid composition; and the lipid composition comprises a lipidoid having structural Formula (I):

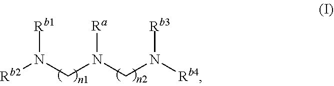

or a pharmaceutically acceptable salt thereof, wherein R^{a}, R^{b1}, R^{b2}, R^{b3}, R^{b4}, n1 and n2 are as described herein.

### RELATED APPLICATIONS

This application claims the benefit of priority to U.S. Provisional Patent Application No. 63/318,971, filed Mar. 11, 2022; the contents of which are hereby incorporated by reference in their entirety.

### GOVERNMENT SUPPORT

This invention was made with government support under grant numbers EB027170 and TR002636 awarded by the National Institutes of Health. The government has certain rights in the invention.

### BACKGROUND

Directed lipid nanoparticles (LNPs) delivery of mRNA and other therapeutic payloads has been limited by the technical challenge of achieving safe, effective, and specific in vivo delivery of mRNA and other therapeutic payloads.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### SUMMARY

Disclosed are lipidoids (or lipids) and lipid nanoparticles (LNPs) comprising them for delivery of mRNA and other therapeutic payloads to the liver. While LNPs have recently been approved for delivery of siRNA to the liver, further innovation is necessary to capture the full therapeutic potential of this remarkable technology.

In one aspect, disclosed is a composition, comprising: a pharmaceutical agent and a lipid composition; wherein the pharmaceutical agent is assembled with the lipid composition; and the lipid composition comprises a lipidoid having structural Formula (I):

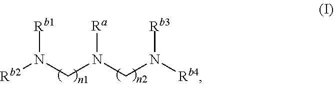

or a pharmaceutically acceptable salt thereof;
wherein:
R^{a} is a substituted or unsubstituted C₁-C₆ alkyl or C₂-C₆ hydroxyalkyl;
n1 and n2 are each independently 1, 2, 3, or 4; and
R^{b1}, R^{b2}, R^{b3} and R^{b4} are each independently

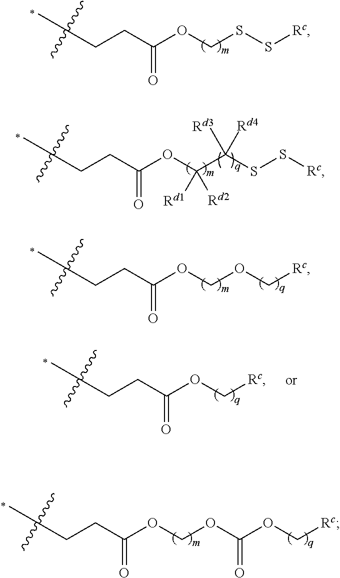

* indicates the point of attachment to N;
each R^{c} is independently an alkyl or an alkenyl;
R^{d1}, R^{d2}, R^{d3} and R^{d4} are each independently H or C₁-C₄ alkyl, wherein at least one of R^{d1}, R^{d2}, R^{d3} and R^{d4} is not H;
each m is independently an integer from 1-10; and
each q is independently an integer from 1-10.

In one aspect, disclosed is a composition, comprising: a pharmaceutical agent and a lipid composition; wherein the pharmaceutical agent is assembled with the lipid composition; and the lipid composition comprises a lipidoid comprising an amine head group and at least one hydrophobic tail R^{Lipid}. In some embodiments, the amine head group has a structure of

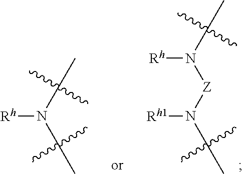

in which R^{h} and R^{h1} independently, is H, C1-20 alkyl, C2-C20 alkenyl, C2-C20 alkynyl, C3-C20 cycloalkyl or heterocycloalkyl, C1-C20 heteroalkyl, C₃-C20 aryl or heteroaryl, or a R^{Lipid}, and Z is a C1-C20 bivalent aliphatic radical, a C1-C20 bivalent heteroaliphatic radical, a bivalent aryl radical, or a bivalent heteroaryl radical. In some embodiments, R^{Lipid} is selected from the group consisting of

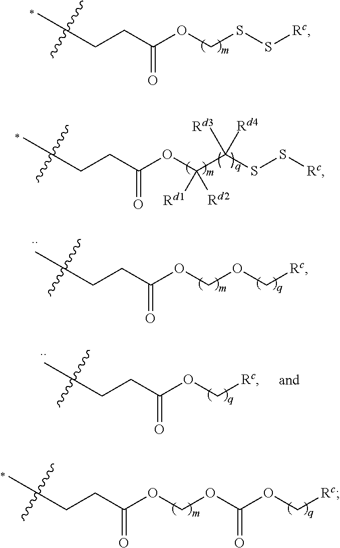

* indicates the point of attachment to N;
each R^{c} is independently an alkyl or an alkenyl;
R^{d1}, R^{d2}, R^{d3} and R^{d4} are each independently H or C₁-C₄ alkyl, wherein at least one of R^{d1}, R^{d2}, R^{d3} and R^{d4} is not H;
each m is independently an integer from 1-10; and
each q is independently an integer from 1-10.

In some embodiments, R^{h} is C1-C10 alkyl. In some embodiments, R^{h} is substituted C1-C10 alkyl. In some embodiments, the substituent is hydroxyl, alkoxy, amine, amide, cycloamine, cyclic amine, guanidine or salt thereof.

In some embodiments, R^{h1} is C1-C10 alkyl. In some embodiments, R^{h1} is substituted C1-C10 alkyl. In some embodiments, the substituent is hydroxyl, alkoxy, amine, amide, cycloamine, cyclic amine, guanidine or salt thereof.

In some embodiments, Z is a C1-C20 bivalent aliphatic radical. In some embodiments, Z is a substituted C₁-C₂₀ bivalent aliphatic radical. In some embodiments, Z is a C1-C20 hydroxyalkyl.

### BRIEF DESCRIPTION OF THE DRAWINGS

FIGS. 1A-1J depict in vivo delivery of f. luciferase mRNA by the various LNPs of the present disclosure. FIGS. 1A-1C show quantification of f. luciferase bioluminescence intensity. FIGS. 1D-1I show whole body bioluminescence from IVIS imaging of adult BALB/c mice at about 6 hours post single i.v. injections of F. luciferase mRNA/LNPs. FIG. 1J is a scheme for synthesizing the ionizable lipids provided herein.

FIGS. 2A-2F shows the in vivo optimization of lipid 88 LNP formulations using 5omU-Fluc mRNA (mLuc). FIG. 2A shows the structure of lipidoids 88 and its TEM image. FIG. 2B shows a schematic illustration of in vivo screening of LNP88 formulations with help lipids (DOPE, DOPC, DSPC) via tail-based intravenous (I.V.) injection. FIG. 2C shows in vivo images of living mice injected with TriLink mLuc loaded a series of lipid 88 formulations, and a commercial lipid ALC0315. Images were taken by IVIS imaging system at 6 hr post I.V. injection. FIG. 2D shows the quantification of expression of Firefly luciferase (Fluc) at the liver-like region of mice, p/sec/cm2/sr. FIG. 2E shows in vivo images of living mice injected with mLuc loaded multiple lipid 88 formulations, and commercial lipid ALC0315 for further screening based on the ratio 16:4:2:1 (L88:Chol:DOPC:DMG-PEG, w w). Images were taken by IVIS imaging system at 6 hr post I.V. injection. FIG. 2F shows the quantification of expression of Fluc at the liver-like region of mice in further screening of lipid 88 formulations (L88:Chol:DOPC:DMG-PEG), p/see/cm2/sr.

FIGS. 3A-3D shows the in vivo optimization of Lipid 88 formulations in mice following intramuscular (I.M.) administration. FIG. 3A shows images of in vivo bioluminescence measurements in mice from 6 hrs, 24 hrs, 48 hrs, and 80 hrs following intramuscular (n=2 mice) injection of mLuc-LNP with varying ratios of helper lipids. FIG. 3B shows the quantification of bioluminescence over time in mice treated with Lipid 88 and 113O12B mRNA/LNP delivery respectively. FIG. 3C shows TEM imaging of the optimized Lipid 88 and 113O12B formulations with and without mRNA loading. Scale represents 100 nm. FIG. 3D shows the quantification of Firefly luciferase expression levels (photo/sec/cm2/sr) in mice with varying ratios of mLuc to LNP88 (1:10-1:30) from 6 hrs to 80 hrs after delivery. ±SD (n=2).

FIGS. 4A-4F shows the biodistribution of LNP88 in neonates via different administrative route. FIG. 4A shows representative images of bioluminescence distribution in neonates (2 days after birth) treated with LNP88/mLuc over time via I.M, I.P and F.V. injection. FIG. 4B shows the quantification of total flux of Fluc luminescence (p/sec/cm²/sr) in neonates over time via I.M, I.P and F.V. injection. FIG. 4C shows the quantification of hepatic expression of Fluc in neonates over time via I.M, I.P and F.V. route. FIG. 4D shows the quantification of thigh expression of Fluc in neonates over time via I.M, I.P and F.V. route. FIG. 4E shows representative ex vivo image of mLuc/LNP88 biodistribution in neonatal organs captured by using the IVIS imaging system at 55 hrs post-delivery (I.M, I.P. and F.V.). FIG. 4F shows quantification of Fluc luminescence (p/sec/cm²/sr) in each organ at 55 hrs.

FIGS. 5A-5G shows spike mRNA UTR and LNP optimization based on T Cell response and IgG titer in mice. FIG. 5A shows the chemical structures of active lipids used for mRNA delivery in this study (Lipid 88, ALC0315, Lipid 65, Lipid 10 and 113O12B). FIG. 5B shows a schematic of UTR and LNP efficacy evaluation, delivering spike mRNA with a range of UTR inserts and LNP formulations via intramuscular injection. Following two doses of spike mRNA/LNP, serum IgG titer was determined by enzyme-linked immunosorbent assay (ELISA) and T cell response was measured by enzyme-linked immunosorbent spot (ELISPOT). FIG. 5C shows representative T cell response ELISpot images for 1 μg mouse cohorts, listing each LNP vehicle and mRNA cargo with LNP88/mRNA formulations circled in black. FIG. 5D shows a comparison of spike mRNA expression in T cells using each 5′UTR-3′UTR combination (a-globin-Mmb-globin, 70 nt (AG)-Apo A-II and Ces1d-AP3B1) based on the spot number of IFN-γ-secreting T cells following LNP88 delivery, 5 mice/group. FIG. 5E shows a comparison of delivery efficiency of LNPs of interest (LNP88, ALC0315, 113012B, L10, L65) for 70 nt (AG)-Spp-Apo A-II mRNA targeting immune cells, 5 mice/group. FIG. 5F shows a comparison of the impact of each UTR combination on mice IgG anti-spike antibody titer, measured by ELISA. FIG. 5G shows a mice IgG anti-spike antibody titer analysis to compare the delivery efficiency of LNPs, measured by ELISA. Significance was statistically performed using a one-way ANOVA nonparametric Brown-Forsythe test. *p<0.05, **p<0.005, ***p<0.0005.

FIGS. 6A-6B shows that LNP88 protects Golden Syrian hamsters from Omicron BQ.1 challenge. FIG. 6A shows the SARS-COV-2 genome copy number in lung tissue at 2 DPI and 4 DPI. FIG. 6B shows the SARS-COV-2 genome copy number in nasal wash at 2 DPI and 4 DPI. Significance was statistically determined by one-way ANOVA nonparametric Brown-Forsythe test, ns, no significance, *p<0.05. Source data are provided as a Source Data file.

FIGS. 7A-7G shows the delivery of mRNA encoding Ug1a1 using lipid 88 (LNP88) in the correction of Cigler-Najjar (CN) syndrome. FIG. 7A shows Ug1a1 activity in liver lysate of WT and mutant mice. FIG. 7B shows a schematic of mUg1a1/LNP88 nanocomplex delivery to neonates with Ug1a1−/− mutation via F.V. and I.P. route. FIG. 7C shows representative images of neonatal growth over time until the Ug1a1−/− mutant died. FIG. 7D shows the weight change (%) monitoring in WT, Ugt1a1−/− mutant and mUgt1a1/LNP88 treated group. FIG. 7E shows the analysis of Ug1a1 activity in liver lysate from WT, Ugt1a1−/− mutant and mUgt1a1/LNP88 treated group. FIG. 7F shows images of neonatal brains. The brain from a 5-day-old WT, Ugt1−/− homozygous neonates are shown compared with a mUg1a1/LNP88 treated Ugt1−/− littermate. FIG. 7G Quantification of bilirubin in neonatal mice brains (5-day-old WT, Ugt1−/− neonates and mUg1a1/LNP88 treated Ugt1−/− littermate). *p<0.05, **p<0.005, ****p<0.00005, ns, no significance.

FIG. 8 shows that delivering mRNA encoding ABE8e and sgRNA_SA or sgRNA_SD (these sgRNAs are compatible with ABE8e and induce splice disruptions in Exon6) for base editing using lipids (306O12B, L88 and L10). Lipid88 showed highest transfection and editing efficiency (almost 60%) among three lipids.

FIG. 9 shows the sizing and zeta potential of fresh LNPs (made of L88) loaded with mRNA encoding ABE8e and sgRNA measured by dynamic light scattering.

FIG. 10 shows a single dose of 3 mg/kg that was compared to a double dose of 3 mg/kg (dose spaced 48 hours apart) of mRNA encoding ABE8e and sgRNA_SA or sgRNA_SD using lipid 88. Results showed that maximal editing was reached with single dose. Significance measured with one way ANOVA. *P<0.05 **P<0.01 ***P<0.001 ****P<0.0001.

FIG. 11 shows the serum levels of angptl3, cholesterol and triglyceride after single administration of LNP (made of L88) loaded with mRNA encoding ABE8e and either sgRNA (sgRNA_SA or sgRNA_SD). Blood taken on day 7. Significant decrease in serum levels observed. Significance measured with one way ANOVA *P<0.05 **P<0.01 ***P<0.001 ****P<0.0001.

FIG. 12 shows the toxicity of treatment that was measured 48 hours post injection of LNP (made of L88) loaded with mRNA encoding ABE8e and sgRNA (sgRNA_SA or sgRNA_SD). Alanine transaminase (ALT), Aspartate transaminase (AST), and Interluekin-6 (IL-6) were measured. No significant increase in toxicity measured. Significance measured with one way ANOVA *P<0.05 **P<0.01 ***P<0.001 ****P<0.0001.

FIG. 13 shows the editing %, ANGPTL3, LDL-C, and Triglycerides 7 days after injection of LNP (made of L88) loaded with mRNA encoding ABE8e and sgRNA (sgRNA_SA or sgRNA_SD). Significance measured by one-way ANOVA.

FIG. 14 shows ANGPTL3, LDL-C, and Triglycerides levels 30 days after injection of LNP (made of L88) loaded with mRNA encoding ABE8e and sgRNA (sgRNA_SA or sgRNA_SD). Significance measured by one-way ANOVA.

FIG. 15 shows LDL-C, 100 days after injection of LNP (made of L88) loaded with mRNA encoding ABE8e and sgRNA (sgRNA_SA or sgRNA_SD). Significance measured by one-way ANOVA.

FIGS. 16A-16G shows the In vivo optimization of mLuc-eGFP 3′UTRs in mice with Ces1d or 70 nt (GG) as the 5′UTR via S.C. route. FIG. 16A shows the overview of UTR optimization in mice. In the mRNA construct, mFLuc-eGFP was capped with CleanCap AG or ARCA cap, tailed with 120 bases of polyadenosine, and Ces1d or 70 nt (GG) as the 5′UTR. The 3′UTR will be inserted with UTRs of interest to assess their performance in vivo. All 5′UTRs and 3′UTRs in this study were screened following in vitro UTR optimization. UTR-modified mFLuc-eGFP was delivered in mice by subcutaneously injection to evaluate the effects of each UTR on Fluc expression. FIG. 16B shows the quality check of mFLuc-eGFP mRNA containing each screened 3′UTR (Mmβ-globin, ha-globin, C3, TIAM1, P450 2E1, AP3B1 and WIPI2) and a consistent 5′UTR (Ces1d). FIG. 16C shows the in vivo bioluminescence levels from 6 hrs to 30 hrs following subcutaneous injection of mLuc-eGFP/LNP88 with Ces1d as the 5′UTR. Scales represent radiance in photons/see/cm2/sr. FIG. 16D shows the quantification of Firefly luciferase (FLuc) expression over time localized in various organ systems (liver, lymph node, and local site). ±SD (n=2). FIG. 16E shows the quality check of mLuc-eGFP mRNA with each screened 3′UTR (Mmβ-globin, MS10433, Apo A-II, ha-globin, AP3B1, POTEE, S0_M_T1012, YY2 TF, OXR1) and a consistent 5′UTR (70 nt (GG)). FIG. 16F shows the bioluminescence measurements from 6 hrs to 30 hrs following subcutaneous injection of mLuc-eGFP/LNP with 70 nt as the 5′UTR. Scale represents radiance in photons/sec/cm²/sr. FIG. 16G shows the quantification of FLuc expression over time at the location of bioluminescence distribution in mice (liver, lymph node and local). ±SD (n=2).

FIGS. 17A-17G demonstrate that LNP88 protects Golden Syrian hamsters from WA1/2020 and Omicron BA.1 challenge. FIG. 17A shows a comparison of delivery efficiency of two batches of LNP88 in mice using mLuc as reporter over time (5 hrs, 28 hrs and 75 hrs). FIG. 17B shows the quantification of FLuc expression levels in radiance (photons/sec/cm²/sr) in mice over time. FIG. 17C shows a schematic of the hamster immunization timeline, including two doses Ces1d-Spp-Ap3B1 mRNA/LNP88 prior to serum collection and SARS-COV-2 challenge. FIG. 17D shows Hamster body weight change (%) following WA1/2020 challenge (4.0×10⁴ PFU/hamster, n=8). FIG. 17E shows hamster body weight change (%) after Omicron BA.1 challenge (1.3×10⁴ PFU/hamster, n=2). FIG. 17F shows a SARS-COV-2 S-specific IgG titer in hamster serum one week post-booster (n=16 per group) serum determined by enzyme-linked immunosorbent assay (ELISA). FIG. 17G shows a comparison of lung viral titers in WA1/2020 or Omicron BA. 1-infected hamsters with or without immunization. Batch I and Batch II were set up to confirm reproducibility. Batch I cohorts include hamsters vaccinated with saline or eSV1 (5 μg/hamster) followed by infection with WA1/2020 (4.0 × 10⁴ PFU/hamster, n=4) and hamsters vaccinated with saline or eSV1 (5 μg/hamster) followed by infection with Omicron BA.1 (1.3×10⁴ PFU/hamster, n=2). Batch I cohorts hamsters vaccinated with saline or eSV1 (5 μg/hamster) followed by infection with WA1/2020 (4.0×10⁴ PFU/hamster, n=5) and hamsters vaccinated with saline or eSV1 (5 μg/hamster) followed by infection with Omicron BA.1 (2.3×10⁴ PFU/hamster, n=5). LOD: 100 pfu/g lung. Significance for FIGS. 17C-17G was statistically determined by one-way ANOVA nonparametric Brown-Forsythe test, ns, no significance, *p<0.05, **p<0.005, ***p<0.0005, ****p<0.00005.

### DETAILED DESCRIPTION

Provided herein are compositions and methods related to lipid nanoparticles (LNPs) comprising ionizable lipids for delivery of nucleic acids (e.g., mRNA and/or sgRNA). In some embodiments, the LNPs comprising the ionizable lipids provided herein have improved in vivo delivery of the nucleic acids compared to LNPs not comprising the ionizable lipids provided herein. In some embodiments, the LNPs comprising the ionizable lipids have more targeted in vivo delivery of the nucleic acids to an organ (e.g., liver, spleen, or lymph nodes) or a cell (e.g., immune cells or hepatocytes) compared to LNPs not comprising the ionizable lipids provided herein.

One aspect of the present disclosure relates to LNPs comprising ionizable lipids that comprise an mRNA encoding a viral antigen (e.g., a spike protein). In some embodiments, the LNPs comprising ionizable lipids can be used for vaccinating against a virus (e.g., a SARS-CoV2 variant of concern, a SARS-CoV2 Omicron variant).

Another aspect of the disclosure relates to LNPs comprising ionizable lipids that comprise an mRNA modified with regulatory sequences (e.g., untranslated regions, UTRs). In some embodiments, the regulatory sequences improve delivery of the mRNA formulated LNPs (e.g., mRNA/LNPs) and/or improve the expression of peptide encoded by the mRNA. In some embodiments, the untranslated regions (UTRs) comprise 5′ UTRs and/or 3′ UTRs. In some cases, the 3′ UTRs comprise Mmβ-globin, ha-globin, C3, TIAM1, P450 2E1, AP3B1, MS10433, Apo A-II, POTEE, S0_M_T1012, YY2 TF, OXR1 or WIPI2, and the 5′ UTRs comprise 70 nt (GG) or Ces1d.

Another aspect of the disclosure relates to LNPs comprising ionizable lipids that comprise an mRNA encoding a therapeutic protein. In some embodiments, the LNPs comprise mRNA encoding UGTLAL for the treatment of Cigler-Najjar (CN) syndrome in a subject.

Another aspect of the disclosure relates to LNPs comprising ionizable lipids that comprise more than one types of nucleic acids. In some embodiments, the LNPs comprise an mRNA encoding a protein for gene editing (e.g. an adenine base editor; e.g., ABE8e) and a short RNA (e.g., sgRNA).

### Compositions

In one aspect, provided herein is a composition comprising a pharmaceutical agent and a lipid composition; wherein the pharmaceutical agent is assembled with the lipid composition; and the lipid composition comprises a lipidoid having structural Formula (I):

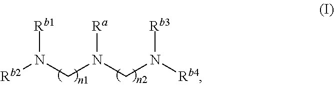

or a pharmaceutically acceptable salt thereof;
wherein:
R^{a} is a substituted or unsubstituted C₁-C₆ alkyl or C₂-C₆ hydroxyalkyl;
n1 and n2 are each independently 1, 2, 3, or 4; and
R^{b1}, R^{b2}, R^{b3} and R^{b4} are each independently

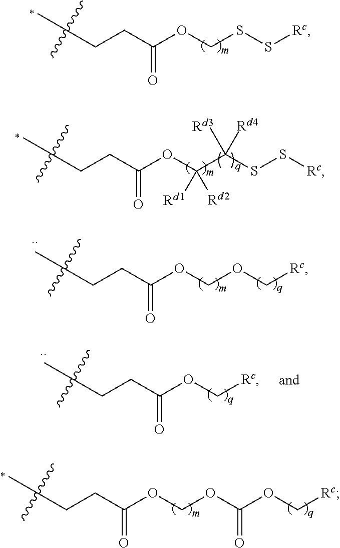

* indicates the point of attachment to N;
each R^{c} is independently an alkyl or an alkenyl;
R^{d1}, R^{d2}, R^{d3} and R^{d4} are each independently H or C₁-C₄ alkyl, wherein at least one of R^{d1}, R^{d2}, R^{d3} and R^{d4} is not H;
each m is independently an integer from 1-10; and
each q is independently an integer from 1-10.

In some embodiments, the composition is for preferential delivery to a target organ or a target cell. In some embodiments, the composition is for modifying an expression profile of a target gene or a gene product thereof in the target organ or the target cell. In some embodiments, the target gene or the gene product is a target protein or a functional variant thereof, or a target transcript. In some embodiments, the pharmaceutical agent is a therapeutic agent, a gene modulating agent, or a vaccine.

In some embodiments, R^{a} is C₁-C₄ alkyl. In some embodiments, R^{a} is C₁-C₃ alkyl. In some embodiments, R^{a} is C₁ alkyl. In some embodiments, R^{a} is substituted C₁-C₄ alkyl. In some embodiments, R^{a} is C₁-C₃ substituted alkyl. In some embodiments, R^{a} is substituted C₁ alkyl.

In some embodiments, R^{a} is C₂-C₅ hydroxyalkyl. In some embodiments, R^{a} is substituted C₂-C₅ hydroxyalkyl.

In some embodiments, n1 is 1, 2 or 3. In some embodiments, n1 is 2 or 3.

In some embodiments, n2 is 1, 2 or 3. In some embodiments, n2 is 2 or 3.

In some embodiments, n1 and n2 are identical.

In some embodiments, at least one of R^{b1}, R^{b2}, R^{b3} and R^{b4} is

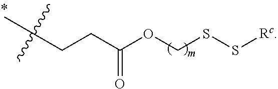

In some embodiments, R^{b1}, R^{b2}, R^{b3} and R^{b4} are each independently

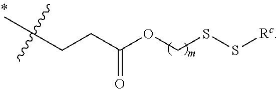

In some embodiments, none of R^{b1}, R^{b2}, R^{b3} and R^{b4} is

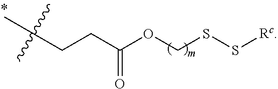

In some embodiments, R^{b1}, R^{b2}, R^{b3} and R^{b4} are each independently

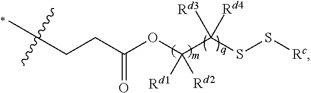

In some embodiments, R^{b1}, R^{b2}, R^{b3} and R^{b4} are each independently

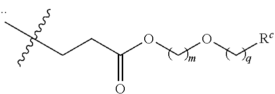

In some embodiments, R^{b1}, R^{b2}, R^{b3} and R^{b4} are each independently

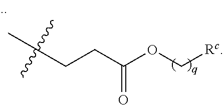

In some embodiments, R^{b1}, R^{b2}, R^{b3} and R^{b4} are each independently

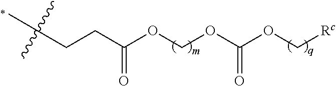

In some embodiments, each R^{c} is independently C₄-C₁₆ alkyl or C₄-C₁₆ alkenyl.

In some embodiments, R^{c} is independently C₄-C₁₂ alkyl or C₄-C₁₂ alkenyl.

In some embodiments, each R^{c} is independently C₄-C₁₆ alkyl. In some embodiments, each R^{c} is independently C₄-C₁₂ alkyl. In some embodiments, each R^{c} is independently C₄-C₁₀ alkyl.

In some embodiments, each R^{c} is independently C₄-C₁₂ alkyl.

In some embodiments, each m is independently 10. In some embodiments, each m is independently 9. In some embodiments, each m is independently 8. In some embodiments, each m is independently an integer from 1 to 8. In some embodiments, each m is independently an integer from 1 to 6. In some embodiments, each m is independently an integer from 1 to 4. In some embodiments, each m is independently an integer from 1, 2, or 3. In some embodiments, each m is independently 1 or 2. In some embodiments, each m is 1.

In some embodiments, each q is independently 10. In some embodiments, each q is independently 9. In some embodiments, each q is independently 8. In some embodiments, each q is independently an integer from 1 to 8. In some embodiments, each q is independently an integer from 1 to 6. In some embodiments, each q is independently an integer from 1 to 4. In some embodiments, each q is independently an integer from 1, 2, or 3. In some embodiments, each q is independently 1 or 2. In some embodiments, each q is 2.

In some embodiments, R^{b1}, R^{b2}, R^{b3} and R^{b4} are each independently

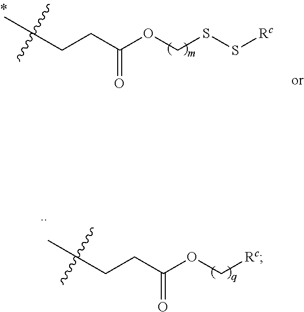

and
at least one of R^{b1}, R^{b2}, R^{b3} and R^{b4} is

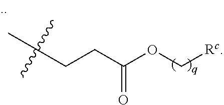

In some embodiments, at least two of R^{b1}, R^{b2}, R^{b3} and R^{b4} are each independently

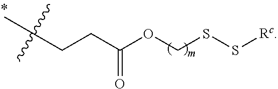

In some embodiments, at least three of R^{b1}, R^{b2}, R^{b3} and R^{b4} are each independently

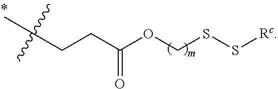

In some embodiments, all four of R^{b1}, R^{b2}, R^{b3} and R^{b4} are each independently

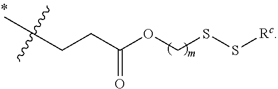

In some embodiments, at least two (e.g., at least three, or all four) of R^{b1}, R^{b2}, R^{b3} and R^{b4} are each independently

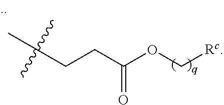

In some embodiments, at least three of R^{b1}, R^{b2}, R^{b3} and R^{b4} are each independently

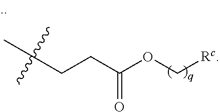

In some embodiments, all four of R^{b1}, R^{b2}, R^{b3} and R^{b4} are each independently

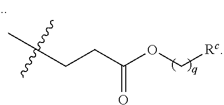

In some embodiments, the lipidoid has structural Formula (II_{A}), (II_{B}), (II_{C}), (II_{D}), (II_{E}), (II_{F}), (II_{G}), or (II_{H}):

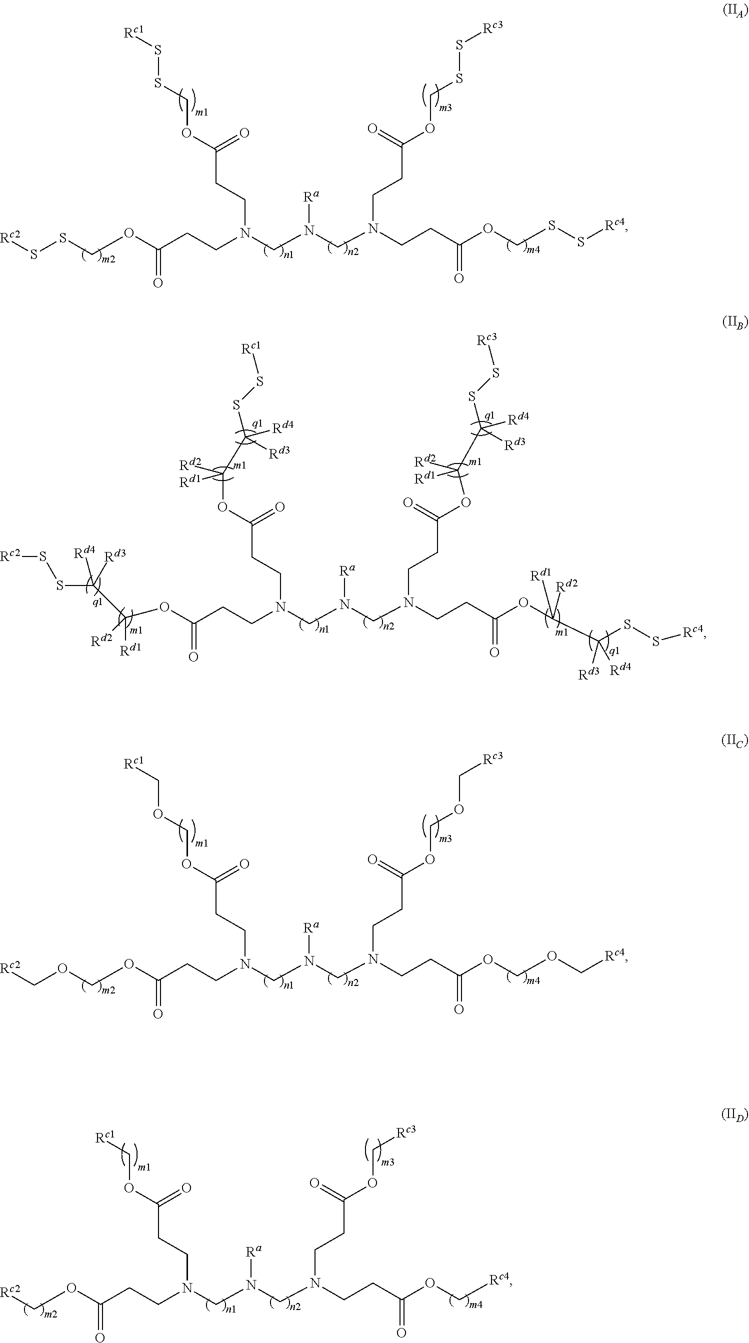

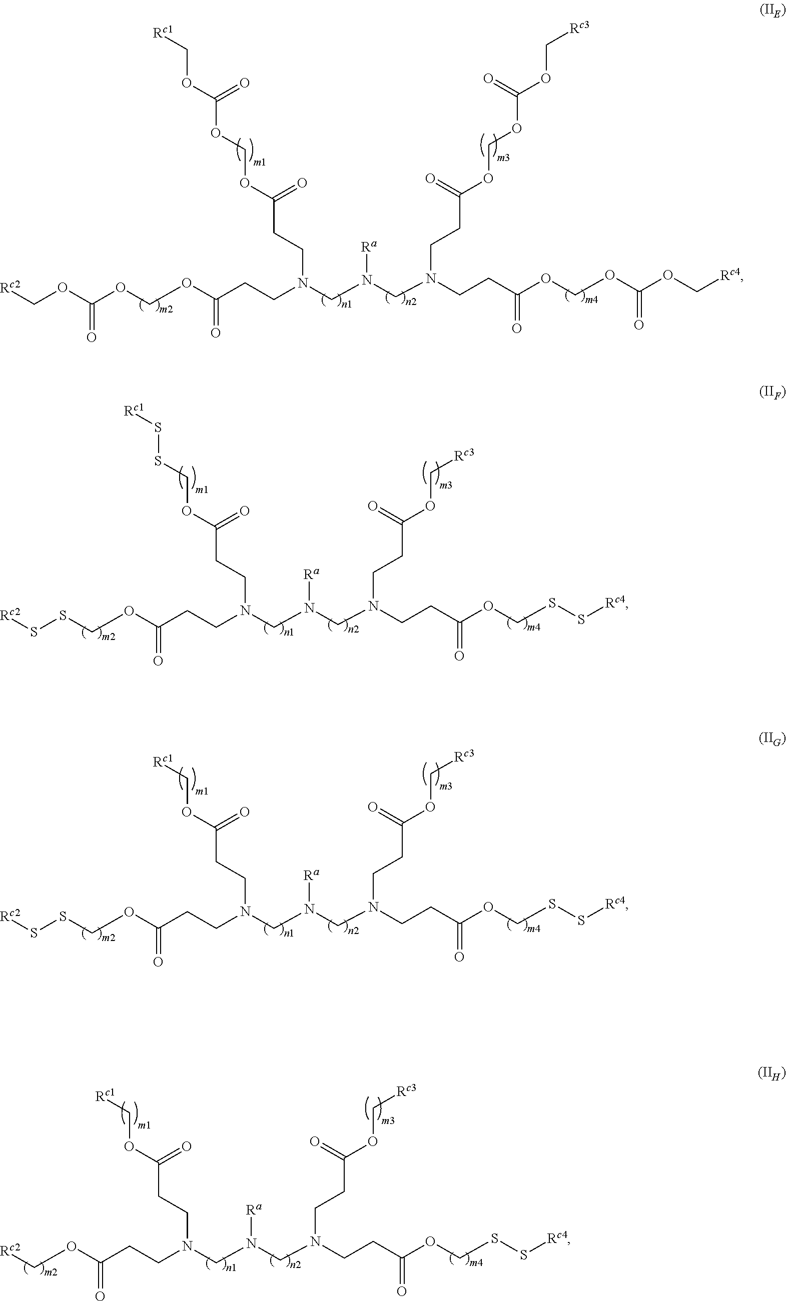

or a pharmaceutically acceptable salt thereof, wherein:
n1 and n2 are each independently 1, 2, 3, or 4;
m1, m2, m3, and m4, when present, are each independently 1, 2, or 3;
q1, q2, q3 and q4, when present, are each independently 1, 2, 3, or 4; and
R^{c1}, R^{c2}, R^{c3} and R^{c4}, when present, are each independently C₄-C₂₀ alkyl or C₄-C₂₀ alkenyl.

In one aspect, disclosed herein is a composition, comprising: a pharmaceutical agent and a lipid composition; wherein the pharmaceutical agent is assembled with the lipid composition; and the lipid composition comprises a lipidoid comprising an amine head group and at least one hydrophobic tail R^{Lipid}. In some embodiments, the amine head group has a structure of

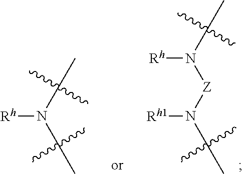

in which R^{h} and R^{h1} independently, is H, C1-20 alkyl, C2-C20 alkenyl, C2-C20 alkynyl, C3-C20 cycloalkyl or heterocycloalkyl, C1-C20 heteroalkyl, C3-C20 aryl or heteroaryl, or a R^{Lipid}, and Z is a C1-C20 bivalent aliphatic radical, a C1-C20 bivalent heteroaliphatic radical, a bivalent aryl radical, or a bivalent heteroaryl radical. In some embodiments, R^{Lipid} is selected from the group consisting of

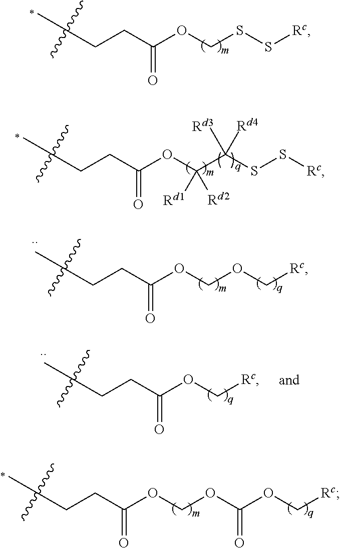

* indicates the point of attachment to N;
each R^{c} is independently an alkyl or an alkenyl;
R^{d1}, R^{d2}, R^{d3} and R^{d4} are each independently H or C₁-C₄ alkyl, wherein at least one of R^{d1}, R^{d2}, R^{d3} and R^{d4} is not H;
each m is independently an integer from 1-10; and
each q is independently an integer from 1-10.

In some embodiments, R^{h} is C1-C10 alkyl. In some embodiments, R^{h} is substituted C1-C10 alkyl. In some embodiments, the substituent is hydroxyl, alkoxy, amine, amide, cycloamine, cyclic amine, guanidine or salt thereof. In some embodiments, R^{h} is R^{Lipid}.

In some embodiments, R^{h1} is C1-C10 alkyl. In some embodiments, R^{h1} is substituted C1-C10 alkyl. In some embodiments, the substituent is hydroxyl, alkoxy, amine, amide, cycloamine, cyclic amine, guanidine or salt thereof. In some embodiments, R^{h1} is R^{Lipid}.

In some embodiments, Z is a C1-C20 bivalent aliphatic radical. In some embodiments, Z is a substituted C1-C20 bivalent aliphatic radical. In some embodiments, Z is a C1-C20 hydroxyalkyl.

In some embodiments, the lipidoid comprises two R^{Lipid} tails independently selected from the group consisting of

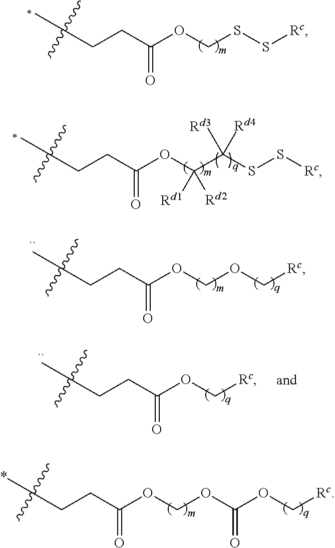

In some embodiments, the lipidoid comprises three R^{Lipid} tails independently selected from the group consisting of

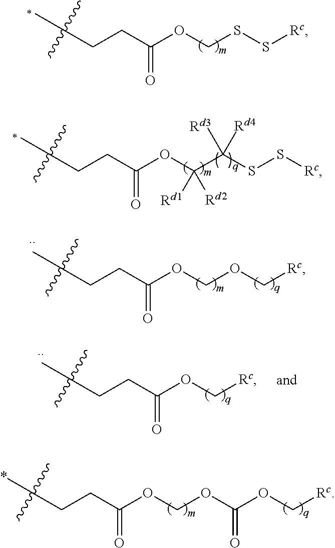

In some embodiments, the lipidoid comprises four R^{Lipid} tails independently selected from the group consisting of

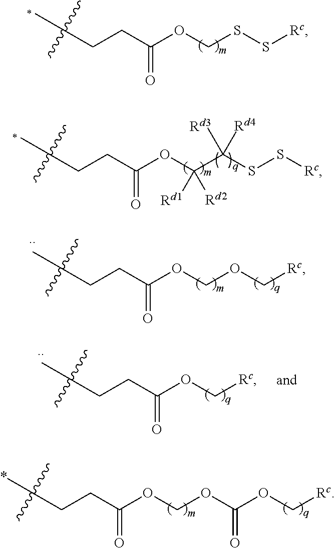

In some embodiments, the amine head group is selected from the TABLE 1.

TABLE 1
Exemplary Amine Head Group

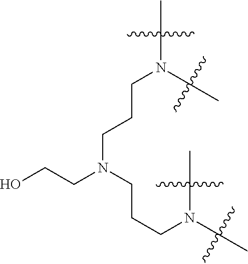 [SMILES: *N(*)CCCN(CCO)CCCN(*)*]

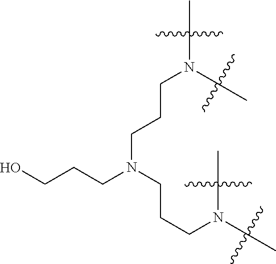 [SMILES: *N(*)CCCN(CCCO)CCCN(*)*]

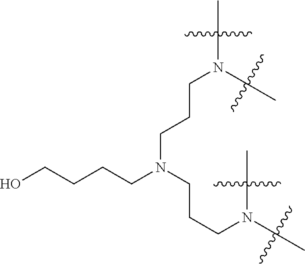 [SMILES: *N(*)CCCN(CCCCO)CCCN(*)*]

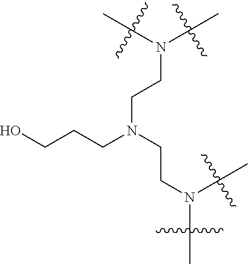 [SMILES: *N(*)CCN(CCCO)CCN(*)*]

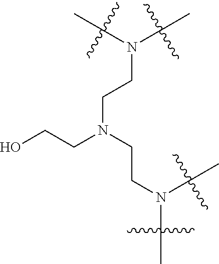 [SMILES: *N(*)CCN(CCO)CCN(*)*]

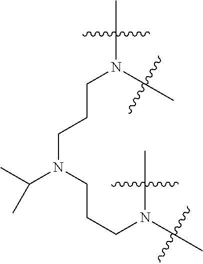 [SMILES: *N(*)CCCN(CCCN(*)*)C(C)C]

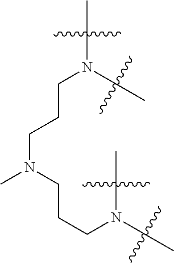 [SMILES: *N(*)CCCN(C)CCCN(*)*]

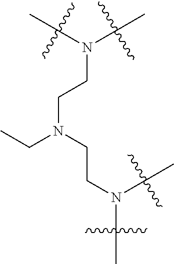 [SMILES: *N(*)CCN(CC)CCN(*)*]

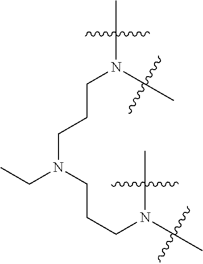 [SMILES: *N(*)CCCN(CC)CCCN(*)*]

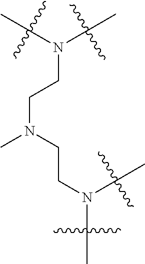 [SMILES: *N(*)CCN(C)CCN(*)*]

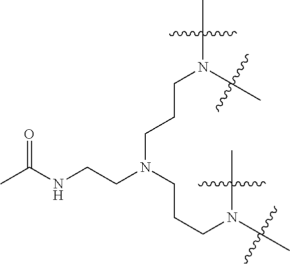 [SMILES: *N(*)CCCN(CCCN(*)*)CCNC(C)=O]

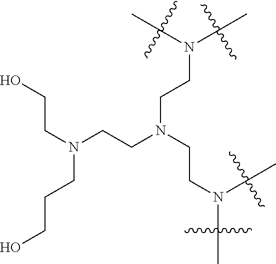 [SMILES: *N(*)CCN(CCN(*)*)CCN(CCO)CCCO]

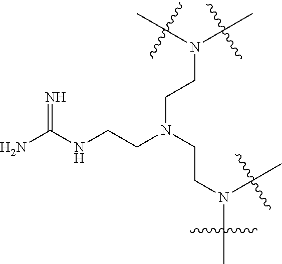 [SMILES: *N(*)CCN(CCNC(=N)N)CCN(*)*]

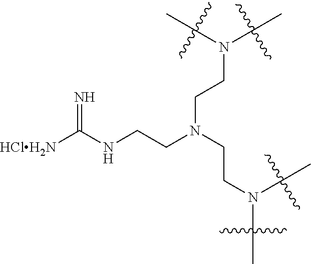

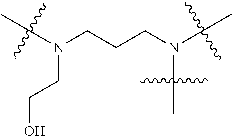 [SMILES: *N(*)CCCN(*)CCO]

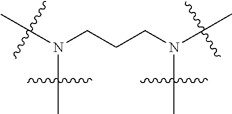 [SMILES: *N(*)CCCN(*)*]

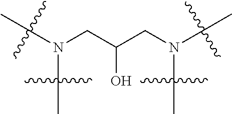 [SMILES: *N(*)CC(O)CN(*)*]

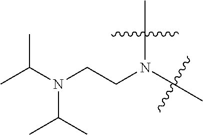 [SMILES: *N(*)CCN(C(C)C)C(C)C]

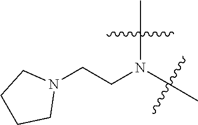 [SMILES: *N(*)CCN1CCCC1]

In some embodiments, the lipidoid is selected from TABLE 2.

TABLE 2
Exemplary Lipidoid

| | |
|---|---|
| 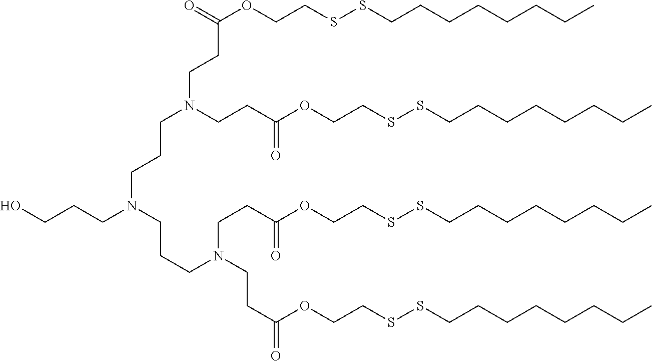 [SMILES: CCCCCCCCSSCCOC(=O)CCN(CCCN(CCCO)CCCN(CCC(=O)OCCSSCCCCCCCC)CCC(=O)OCCSSCCCCCCCC)CCC(=O)OCCSSCCCCCCCC] | 1 |
| 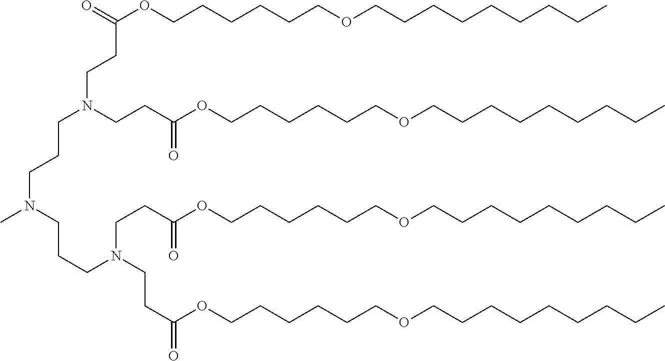 [SMILES: CCCCCCCCCOCCCCCCOC(=O)CCN(CCCN(C)CCCN(CCC(=O)OCCCCCCOCCCCCCCCC)CCC(=O)OCCCCCCOCCCCCCCCC)CCC(=O)OCCCCCCOCCCCCCCCC] | 2 |
| 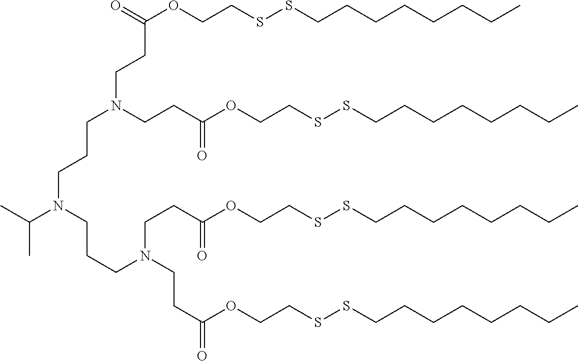 [SMILES: CCCCCCCCSSCCOC(=O)CCN(CCCN(CCCN(CCC(=O)OCCSSCCCCCCCC)CCC(=O)OCCSSCCCCCCCC)C(C)C)CCC(=O)OCCSSCCCCCCCC] | 3 |
| 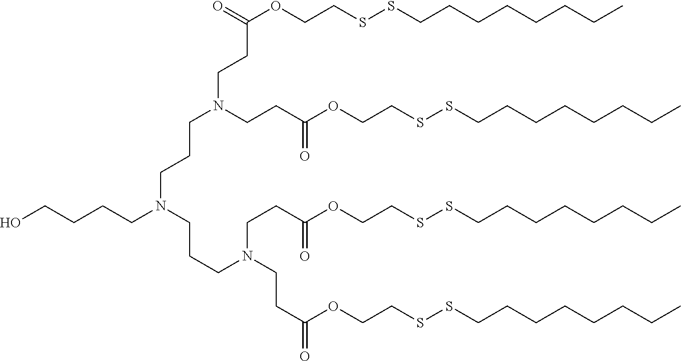 [SMILES: CCCCCCCCSSCCOC(=O)CCN(CCCN(CCCCO)CCCN(CCC(=O)OCCSSCCCCCCCC)CCC(=O)OCCSSCCCCCCCC)CCC(=O)OCCSSCCCCCCCC] | 4 |
| 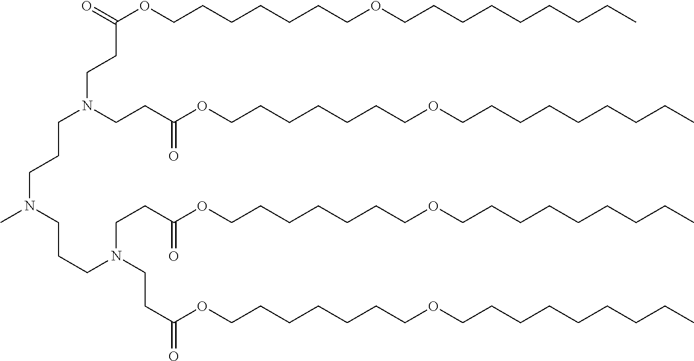 [SMILES: CCCCCCCCCOCCCCCCCOC(=O)CCN(CCCN(C)CCCN(CCC(=O)OCCCCCCCOCCCCCCCCC)CCC(=O)OCCCCCCCOCCCCCCCCC)CCC(=O)OCCCCCCCOCCCCCCCCC] | 5 |
| 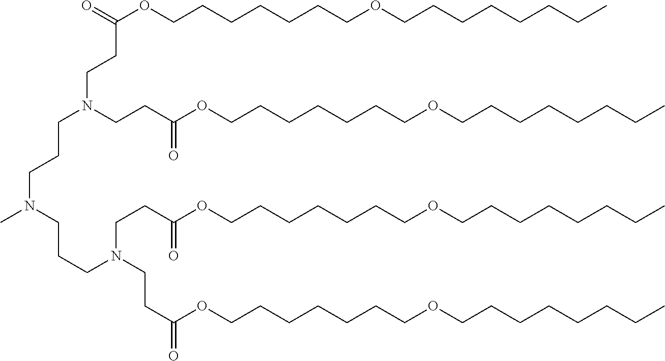 [SMILES: CCCCCCCCOCCCCCCCOC(=O)CCN(CCCN(C)CCCN(CCC(=O)OCCCCCCCOCCCCCCCC)CCC(=O)OCCCCCCCOCCCCCCCC)CCC(=O)OCCCCCCCOCCCCCCCC] | 6 |
| 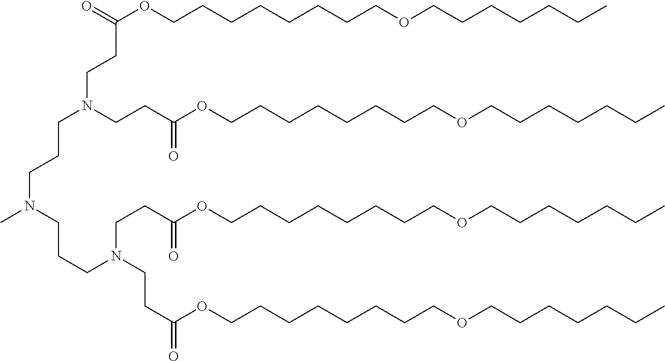 [SMILES: CCCCCCCOCCCCCCCCOC(=O)CCN(CCCN(C)CCCN(CCC(=O)OCCCCCCCCOCCCCCCC)CCC(=O)OCCCCCCCCOCCCCCCC)CCC(=O)OCCCCCCCCOCCCCCCC] | 7 |
| 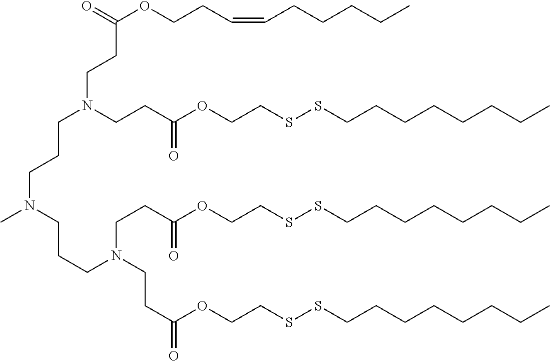 [SMILES: CCCCC/C=C\CCOC(=O)CCN(CCCN(C)CCCN(CCC(=O)OCCSSCCCCCCCC)CCC(=O)OCCSSCCCCCCCC)CCC(=O)OCCSSCCCCCCCC] | 8 |
| 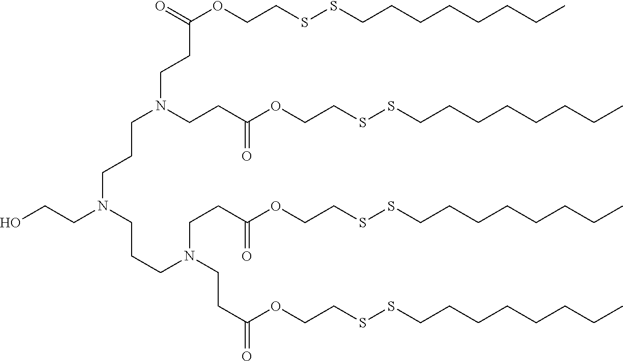 [SMILES: CCCCCCCCSSCCOC(=O)CCN(CCCN(CCO)CCCN(CCC(=O)OCCSSCCCCCCCC)CCC(=O)OCCSSCCCCCCCC)CCC(=O)OCCSSCCCCCCCC] | 9 |
| 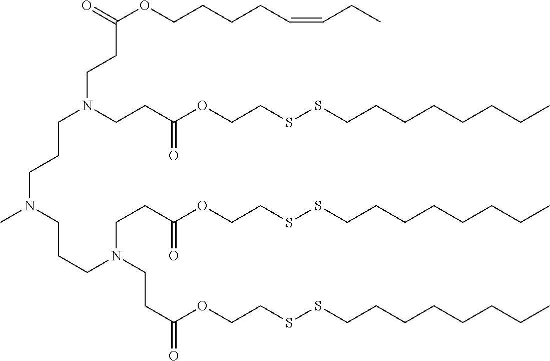 [SMILES: CC/C=C\CCCCOC(=O)CCN(CCCN(C)CCCN(CCC(=O)OCCSSCCCCCCCC)CCC(=O)OCCSSCCCCCCCC)CCC(=O)OCCSSCCCCCCCC] | 10 |
| 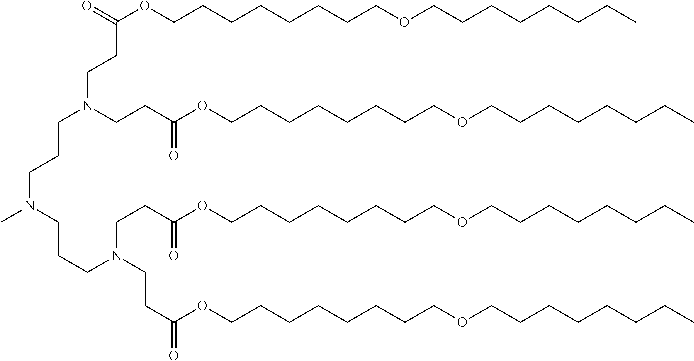 [SMILES: CCCCCCCCOCCCCCCCCOC(=O)CCN(CCCN(C)CCCN(CCC(=O)OCCCCCCCCOCCCCCCCC)CCC(=O)OCCCCCCCCOCCCCCCCC)CCC(=O)OCCCCCCCCOCCCCCCCC] | 11 |
| 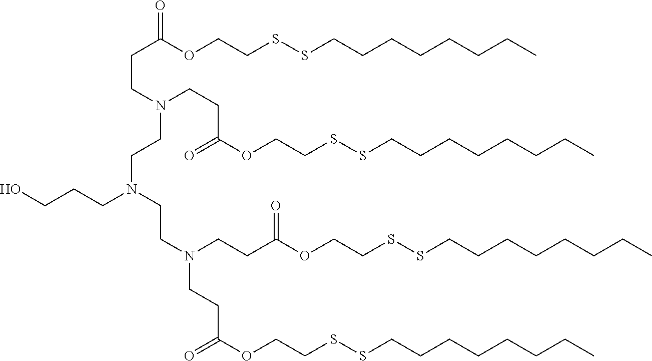 [SMILES: CCCCCCCCSSCCOC(=O)CCN(CCC(=O)OCCSSCCCCCCCC)CCN(CCCO)CCN(CCC(=O)OCCSSCCCCCCCC)CCC(=O)OCCSSCCCCCCCC] | 12 |
| 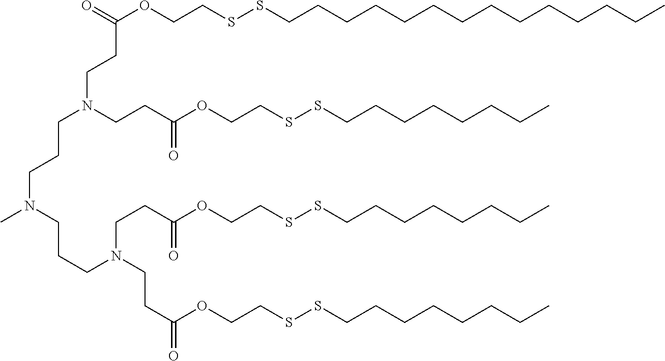 [SMILES: CCCCCCCCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCC] | 13 |
| 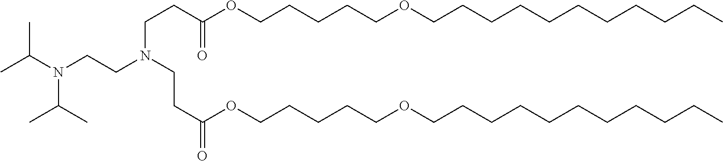 [SMILES: CCCCCCCCCCCOCCCCCOC(=O)CCN(CCC(=O)OCCCCCOCCCCCCCCCCC)CCN(C(C)C)C(C)C] | 14 |
| 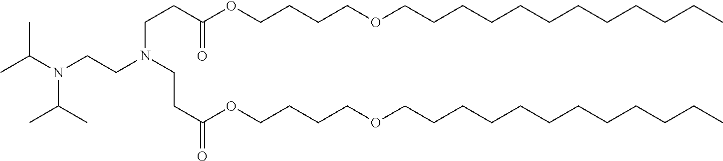 [SMILES: CCCCCCCCCCCCOCCCCOC(=O)CCN(CCC(=O)OCCCCOCCCCCCCCCCCC)CCN(C(C)C)C(C)C] | 15 |
| 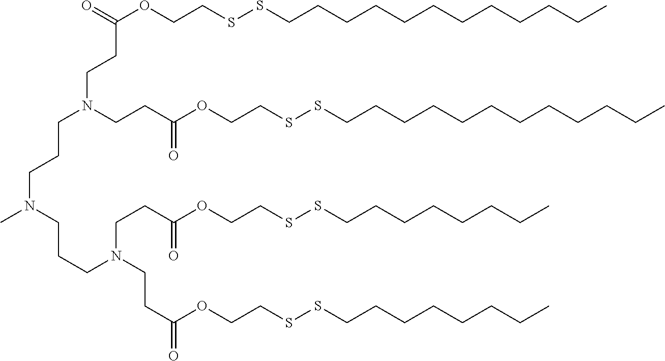 [SMILES: CCCCCCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCCCCCC] | 16 |
| 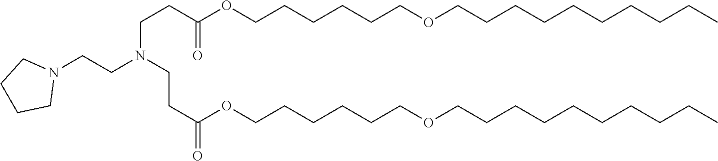 [SMILES: CCCCCCCCCCOCCCCCCOC(=O)CCN(CCC(=O)OCCCCCCOCCCCCCCCCC)CCN1CCCC1] | 17 |
| 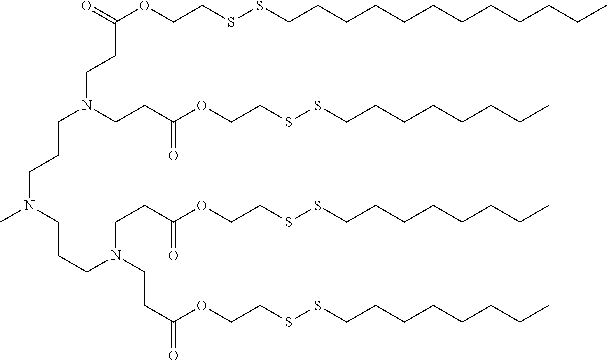 [SMILES: CCCCCCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCC] | 18 |
| 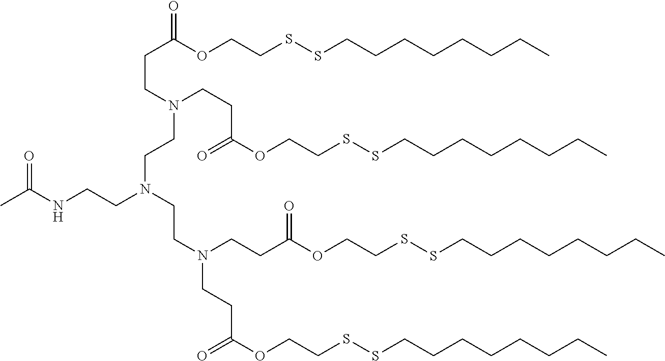 [SMILES: CCCCCCCCSSCCOC(=O)CCN(CCC(=O)OCCSSCCCCCCCC)CCN(CCNC(C)=O)CCN(CCC(=O)OCCSSCCCCCCCC)CCC(=O)OCCSSCCCCCCCC] | 19 |
| 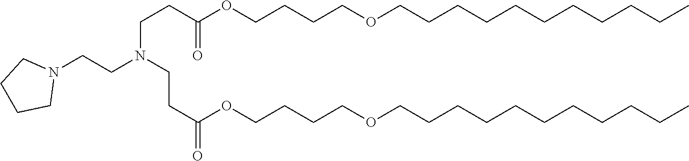 [SMILES: CCCCCCCCCCCOCCCCOC(=O)CCN(CCC(=O)OCCCCOCCCCCCCCCCC)CCN1CCCC1] | 20 |
| 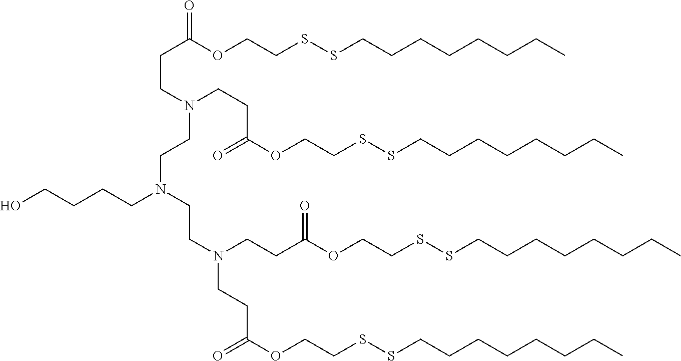 [SMILES: CCCCCCCCSSCCOC(=O)CCN(CCC(=O)OCCSSCCCCCCCC)CCN(CCCCO)CCN(CCC(=O)OCCSSCCCCCCCC)CCC(=O)OCCSSCCCCCCCC] | 21 |
| 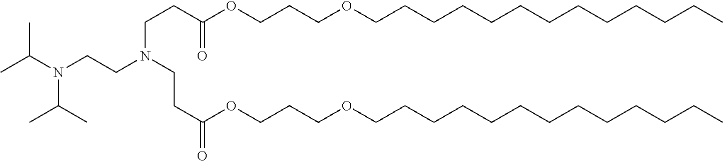 [SMILES: CCCCCCCCCCCCCOCCCOC(=O)CCN(CCC(=O)OCCCOCCCCCCCCCCCCC)CCN(C(C)C)C(C)C] | 22 |
| 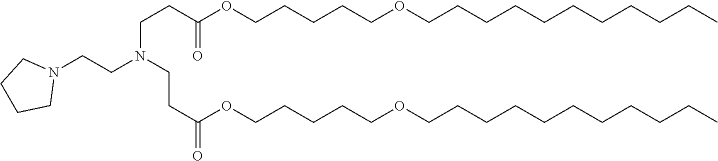 [SMILES: CCCCCCCCCCCOCCCCCOC(=O)CCN(CCC(=O)OCCCCCOCCCCCCCCCCC)CCN1CCCC1] | 23 |
| 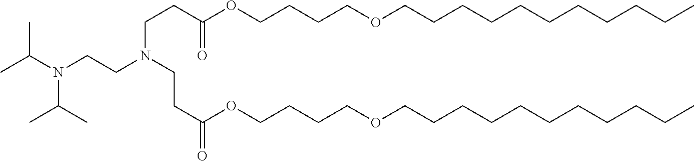 [SMILES: CCCCCCCCCCCOCCCCOC(=O)CCN(CCC(=O)OCCCCOCCCCCCCCCCC)CCN(C(C)C)C(C)C] | 24 |
| 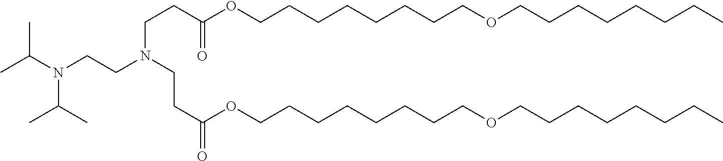 [SMILES: CCCCCCCCOCCCCCCCCOC(=O)CCN(CCC(=O)OCCCCCCCCOCCCCCCCC)CCN(C(C)C)C(C)C] | 25 |
| 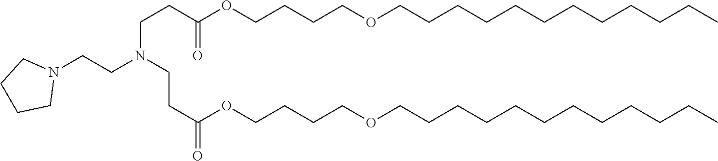 [SMILES: CCCCCCCCCCCCOCCCCOC(=O)CCN(CCC(=O)OCCCCOCCCCCCCCCCCC)CCN1CCCC1] | 26 |
| 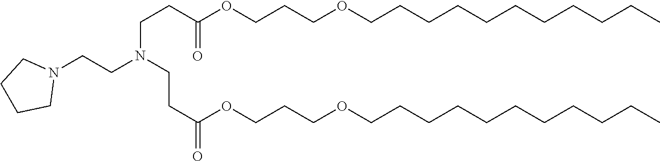 [SMILES: CCCCCCCCCCCOCCCOC(=O)CCN(CCC(=O)OCCCOCCCCCCCCCCC)CCN1CCCC1] | 27 |
| 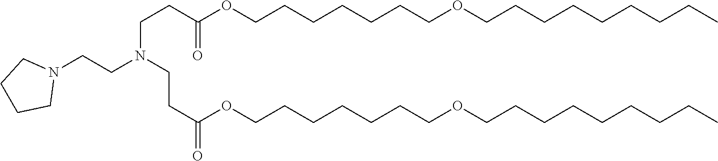 [SMILES: CCCCCCCCCOCCCCCCCOC(=O)CCN(CCC(=O)OCCCCCCCOCCCCCCCCC)CCN1CCCC1] | 28 |
| 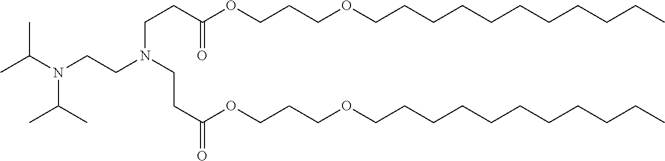 [SMILES: CCCCCCCCCCCOCCCOC(=O)CCN(CCC(=O)OCCCOCCCCCCCCCCC)CCN(C(C)C)C(C)C] | 29 |
| 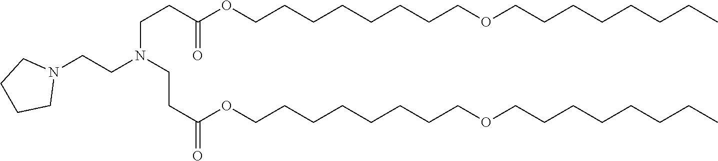 [SMILES: CCCCCCCCOCCCCCCCCOC(=O)CCN(CCC(=O)OCCCCCCCCOCCCCCCCC)CCN1CCCC1] | 30 |
| 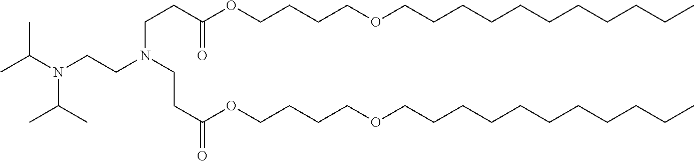 [SMILES: CCCCCCCCCCCOCCCCOC(=O)CCN(CCC(=O)OCCCCOCCCCCCCCCCC)CCN(C(C)C)C(C)C] | 31 |
| 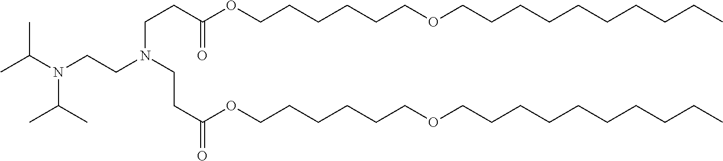 [SMILES: CCCCCCCCCCOCCCCCCOC(=O)CCN(CCC(=O)OCCCCCCOCCCCCCCCCC)CCN(C(C)C)C(C)C] | 32 |
| 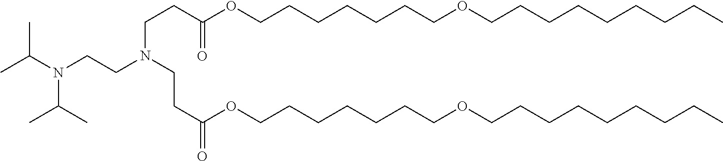 [SMILES: CCCCCCCCCOCCCCCCCOC(=O)CCN(CCC(=O)OCCCCCCCOCCCCCCCCC)CCN(C(C)C)C(C)C] | 33 |
| 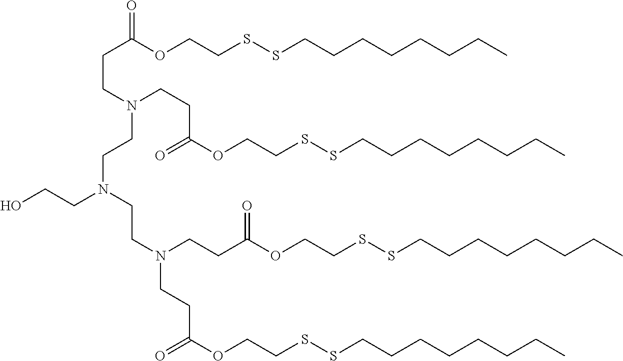 [SMILES: CCCCCCCCSSCCOC(=O)CCN(CCC(=O)OCCSSCCCCCCCC)CCN(CCO)CCN(CCC(=O)OCCSSCCCCCCCC)CCC(=O)OCCSSCCCCCCCC] | 34 |
| 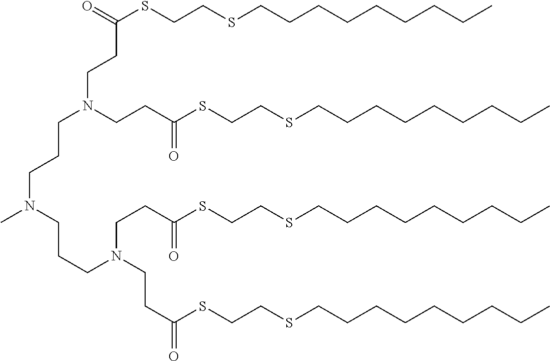 [SMILES: CCCCCCCCCSCCSC(=O)CCN(CCCN(C)CCCN(CCC(=O)SCCSCCCCCCCCC)CCC(=O)SCCSCCCCCCCCC)CCC(=O)SCCSCCCCCCCCC] | 35 |
| 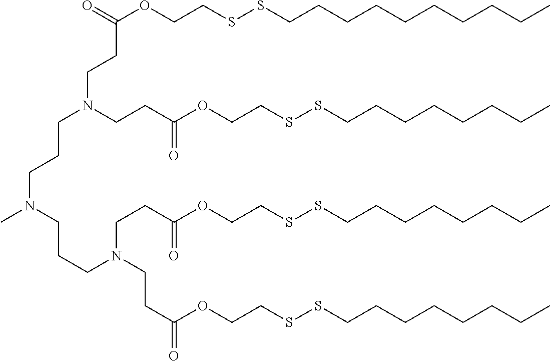 [SMILES: CCCCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCC] | 36 |
| 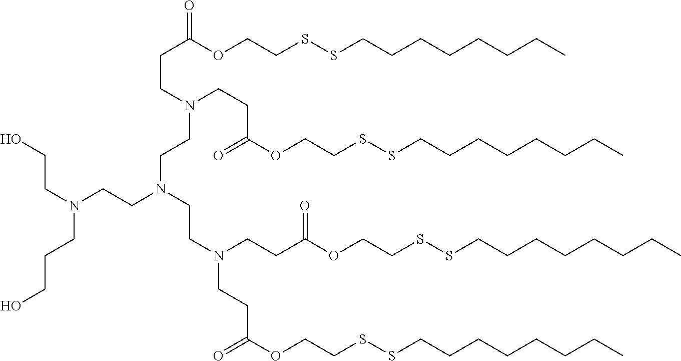 [SMILES: CCCCCCCCSSCCOC(=O)CCN(CCC(=O)OCCSSCCCCCCCC)CCN(CCN(CCO)CCCO)CCN(CCC(=O)OCCSSCCCCCCCC)CCC(=O)OCCSSCCCCCCCC] | 37 |
| 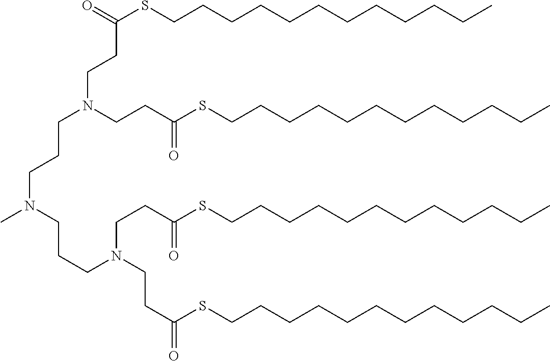 [SMILES: CCCCCCCCCCCCSC(=O)CCN(CCCN(C)CCCN(CCC(=O)SCCCCCCCCCCCC)CCC(=O)SCCCCCCCCCCCC)CCC(=O)SCCCCCCCCCCCC] | 38 |
| 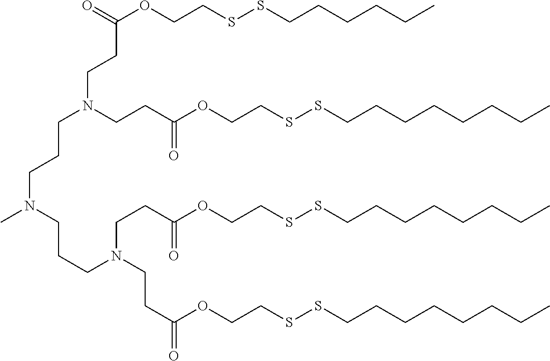 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCC] | 39 |
| 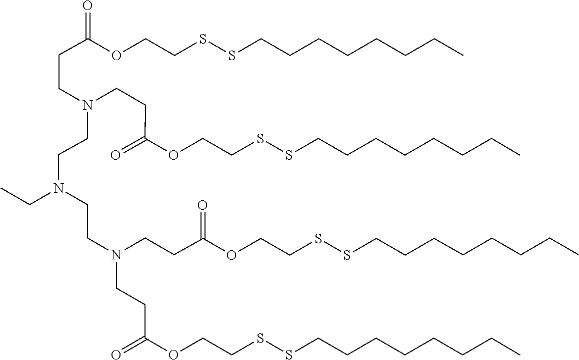 [SMILES: CCCCCCCCSSCCOC(=O)CCN(CCC(=O)OCCSSCCCCCCCC)CCN(CC)CCN(CCC(=O)OCCSSCCCCCCCC)CCC(=O)OCCSSCCCCCCCC] | 40 |
| 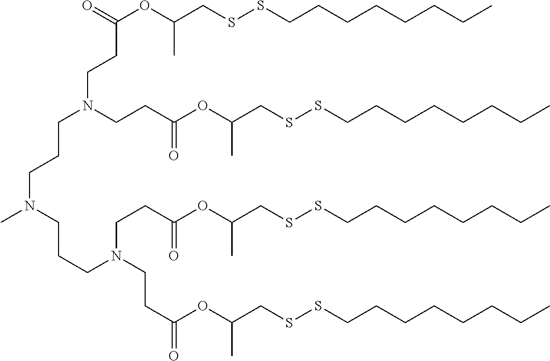 [SMILES: CCCCCCCCSSCC(C)OC(=O)CCN(CCCN(C)CCCN(CCC(=O)OC(C)CSSCCCCCCCC)CCC(=O)OC(C)CSSCCCCCCCC)CCC(=O)OC(C)CSSCCCCCCCC] | 41 |
| 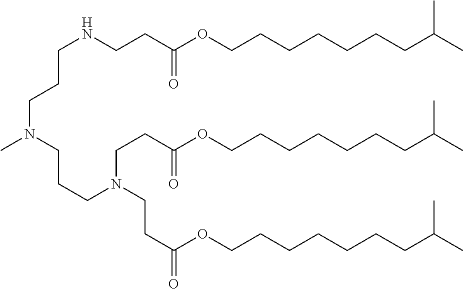 [SMILES: CC(C)CCCCCCCOC(=O)CCNCCCN(C)CCCN(CCC(=O)OCCCCCCCC(C)C)CCC(=O)OCCCCCCCC(C)C] | 42 |
| 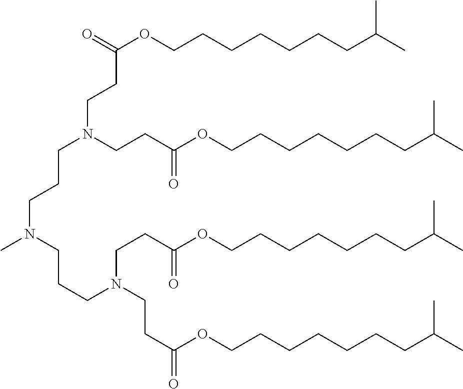 [SMILES: CC(C)CCCCCCCOC(=O)CCN(CCCN(C)CCCN(CCC(=O)OCCCCCCCC(C)C)CCC(=O)OCCCCCCCC(C)C)CCC(=O)OCCCCCCCC(C)C] | 43A |
| 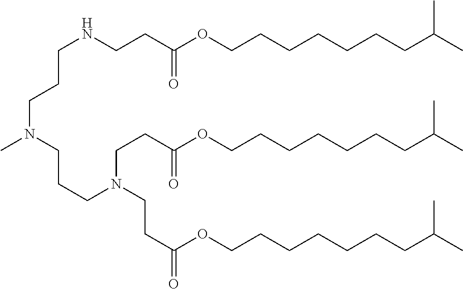 [SMILES: CC(C)CCCCCCCOC(=O)CCNCCCN(C)CCCN(CCC(=O)OCCCCCCCC(C)C)CCC(=O)OCCCCCCCC(C)C] | 43B |
| 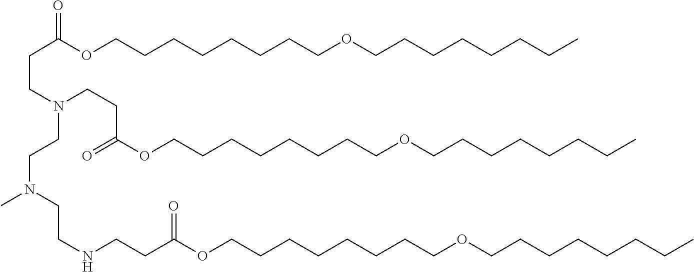 [SMILES: CCCCCCCCOCCCCCCCCOC(=O)CCNCCN(C)CCN(CCC(=O)OCCCCCCCCOCCCCCCCC)CCC(=O)OCCCCCCCCOCCCCCCCC] | 44 |
| 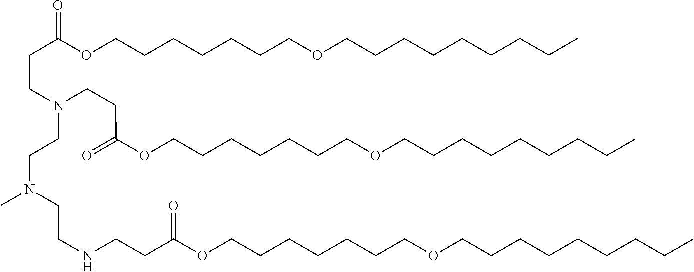 [SMILES: CCCCCCCCCOCCCCCCCOC(=O)CCNCCN(C)CCN(CCC(=O)OCCCCCCCOCCCCCCCCC)CCC(=O)OCCCCCCCOCCCCCCCCC] | 45 |
| 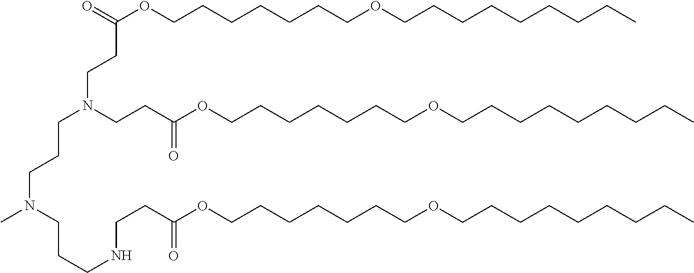 [SMILES: CCCCCCCCCOCCCCCCCOC(=O)CCNCCCN(C)CCCN(CCC(=O)OCCCCCCCOCCCCCCCCC)CCC(=O)OCCCCCCCOCCCCCCCCC] | 46 |
| 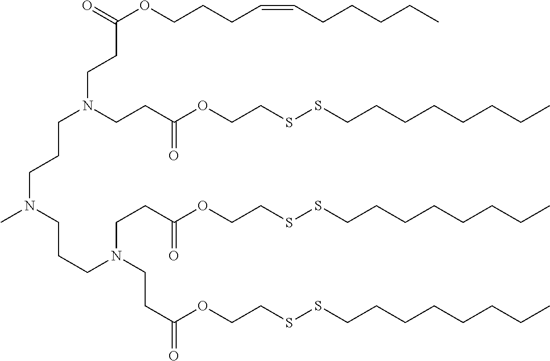 [SMILES: CCCCC/C=C\CCCOC(=O)CCN(CCCN(C)CCCN(CCC(=O)OCCSSCCCCCCCC)CCC(=O)OCCSSCCCCCCCC)CCC(=O)OCCSSCCCCCCCC] | 47 |
| 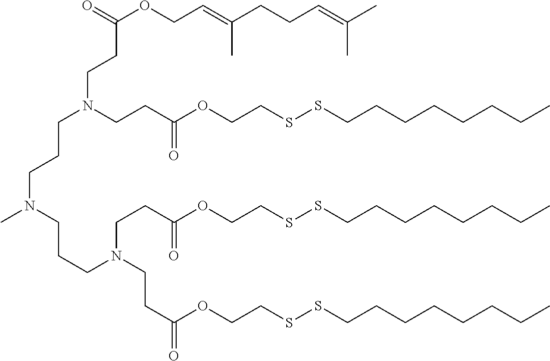 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OC/C=C(\C)CCC=C(C)C] | 48 |
| 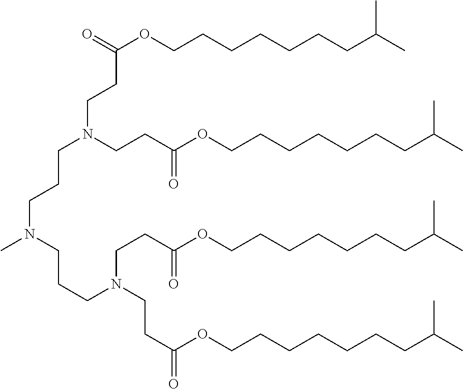 [SMILES: CC(C)CCCCCCCOC(=O)CCN(CCCN(C)CCCN(CCC(=O)OCCCCCCCC(C)C)CCC(=O)OCCCCCCCC(C)C)CCC(=O)OCCCCCCCC(C)C] | 49 |
| 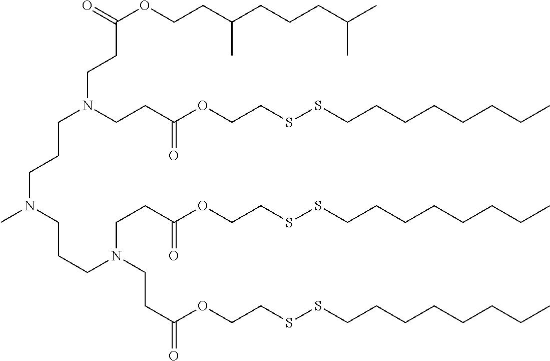 [SMILES: CCCCCCCCSSCCOC(=O)CCN(CCCN(C)CCCN(CCC(=O)OCCSSCCCCCCCC)CCC(=O)OCCC(C)CCCC(C)C)CCC(=O)OCCSSCCCCCCCC] | 50 |
| 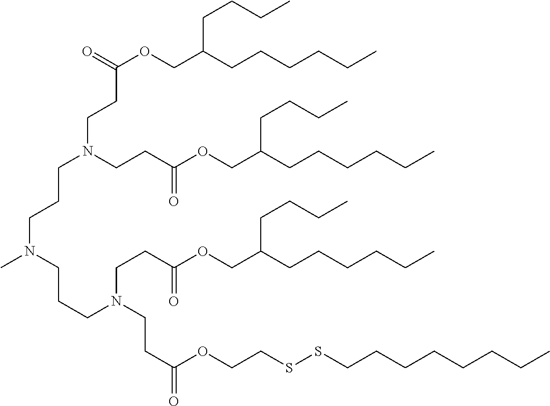 [SMILES: CCCCCCCCSSCCOC(=O)CCN(CCCN(C)CCCN(CCC(=O)OCC(CCCC)CCCCCC)CCC(=O)OCC(CCCC)CCCCCC)CCC(=O)OCC(CCCC)CCCCCC] | 51 |
| 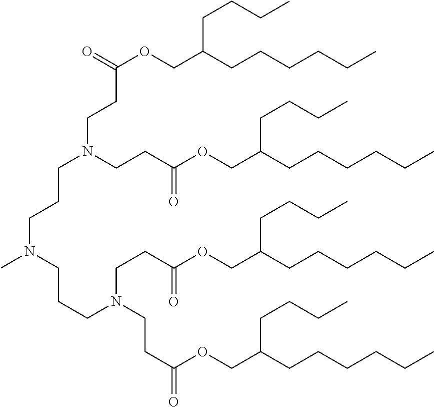 [SMILES: CCCCCCC(CCCC)COC(=O)CCN(CCCN(C)CCCN(CCC(=O)OCC(CCCC)CCCCCC)CCC(=O)OCC(CCCC)CCCCCC)CCC(=O)OCC(CCCC)CCCCCC] | 52 |
| 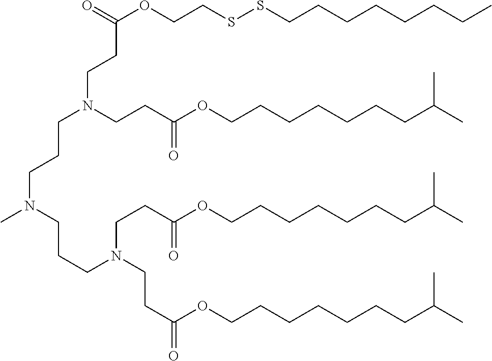 [SMILES: CCCCCCCCSSCCOC(=O)CCN(CCCN(C)CCCN(CCC(=O)OCCCCCCCC(C)C)CCC(=O)OCCCCCCCC(C)C)CCC(=O)OCCCCCCCC(C)C] | 53 |
| 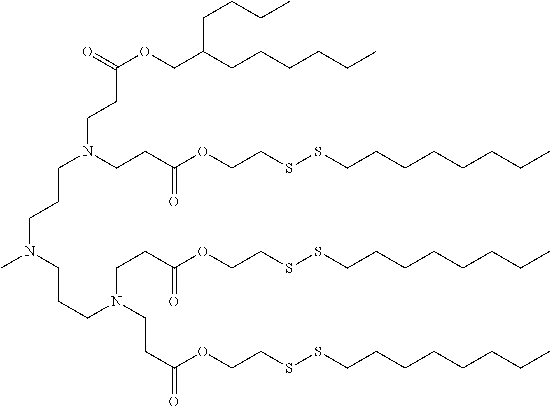 [SMILES: CCCCCCCCSSCCOC(=O)CCN(CCCN(C)CCCN(CCC(=O)OCCSSCCCCCCCC)CCC(=O)OCC(CCCC)CCCCCC)CCC(=O)OCCSSCCCCCCCC] | 54 |
| 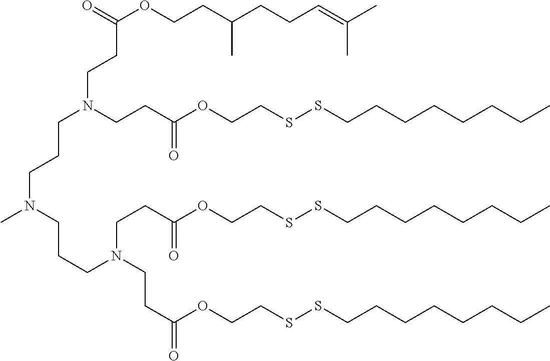 [SMILES: CCCCCCCCSSCCOC(=O)CCN(CCCN(C)CCCN(CCC(=O)OCCSSCCCCCCCC)CCC(=O)OCCC(C)CCC=C(C)C)CCC(=O)OCCSSCCCCCCCC] | 55 |
| 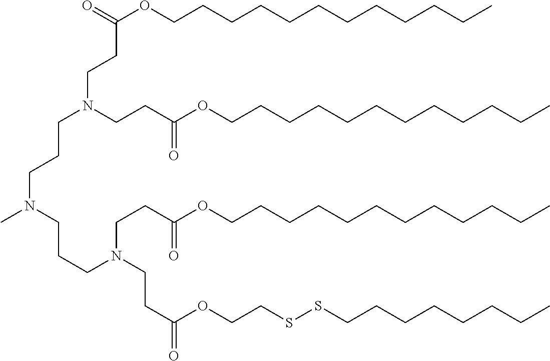 [SMILES: CCCCCCCCCCCCOC(=O)CCN(CCCN(C)CCCN(CCC(=O)OCCCCCCCCCCCC)CCC(=O)OCCSSCCCCCCCC)CCC(=O)OCCCCCCCCCCCC] | 56 |
| 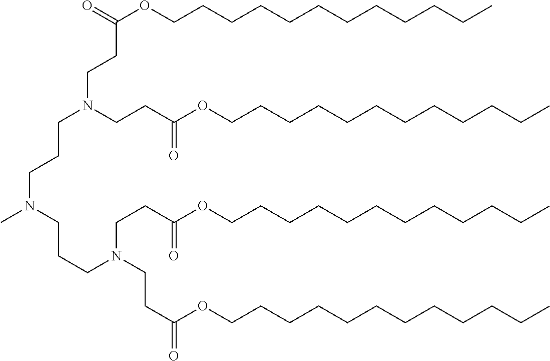 [SMILES: CCCCCCCCCCCCOC(=O)CCN(CCCN(C)CCCN(CCC(=O)OCCCCCCCCCCCC)CCC(=O)OCCCCCCCCCCCC)CCC(=O)OCCCCCCCCCCCC] | 57 |
| 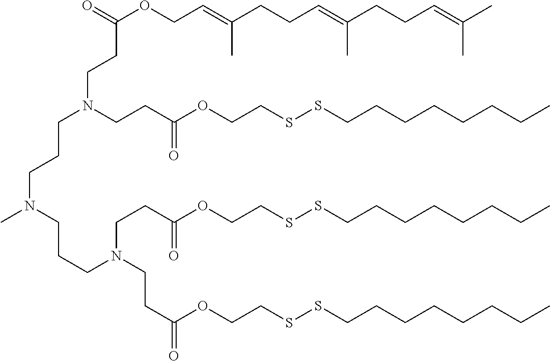 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OC/C=C(\C)CC/C=C(\C)CCC=C(C)C] | 58 |
| 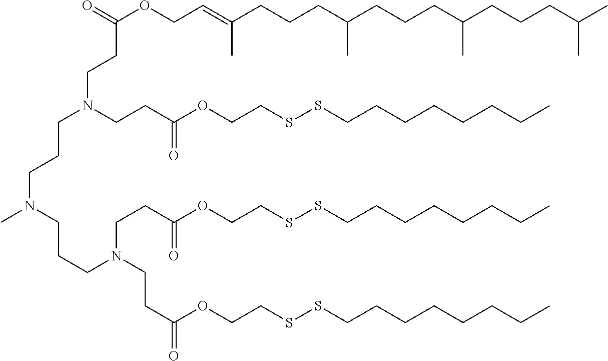 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OC/C=C(\C)CCCC(C)CCCC(C)CCCC(C)C] | 59 |
| 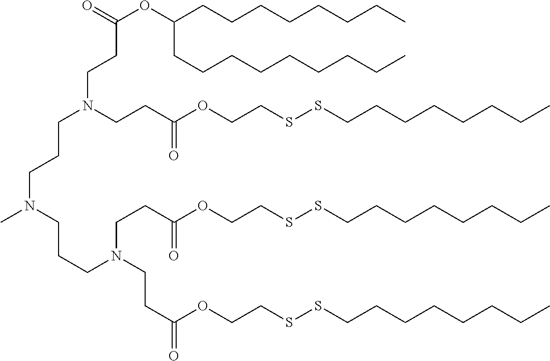 [SMILES: CCCCCCCCCC(CCCCCCCC)OC(=O)CCN(CCCN(C)CCCN(CCC(=O)OCCSSCCCCCCCC)CCC(=O)OCCSSCCCCCCCC)CCC(=O)OCCSSCCCCCCCC] | 60 |
| 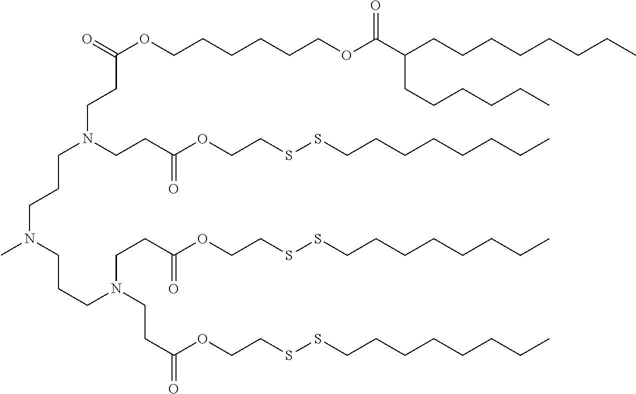 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OCCCCCCOC(=O)C(CCCCCC)CCCCCCCC] | 61 |
| 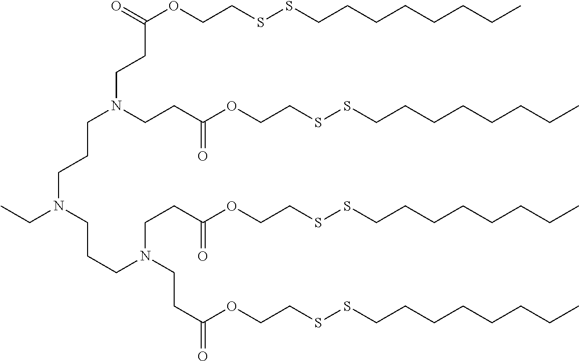 [SMILES: CCCCCCCCSSCCOC(=O)CCN(CCCN(CC)CCCN(CCC(=O)OCCSSCCCCCCCC)CCC(=O)OCCSSCCCCCCCC)CCC(=O)OCCSSCCCCCCCC] | 62 |
| 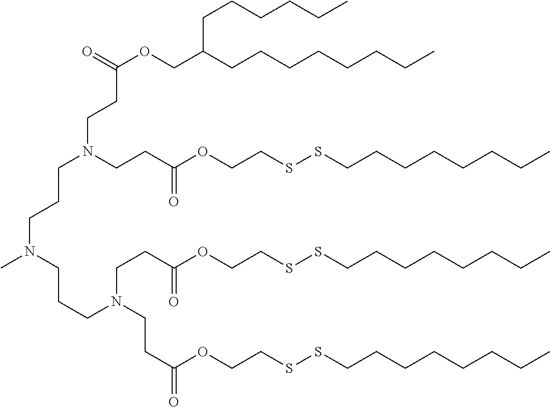 [SMILES: CCCCCCCCSSCCOC(=O)CCN(CCCN(C)CCCN(CCC(=O)OCCSSCCCCCCCC)CCC(=O)OCC(CCCCCC)CCCCCCCC)CCC(=O)OCCSSCCCCCCCC] | 64 |
| 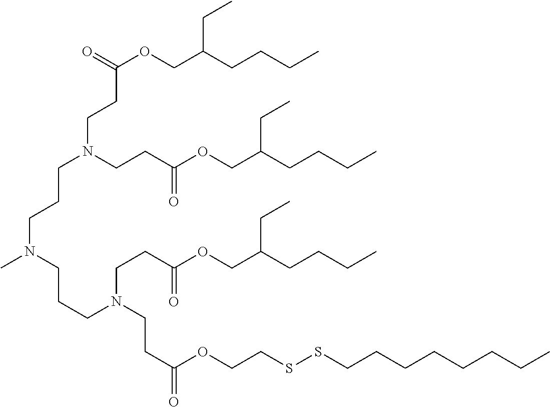 [SMILES: CCCCCCCCSSCCOC(=O)CCN(CCCN(C)CCCN(CCC(=O)OCC(CC)CCCC)CCC(=O)OCC(CC)CCCC)CCC(=O)OCC(CC)CCCC] | 65 |
| 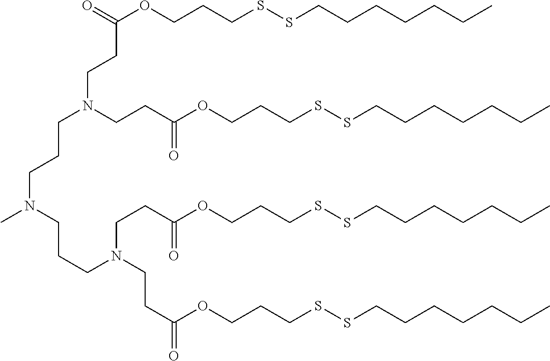 [SMILES: CCCCCCCSSCCCOC(=O)CCN(CCCN(C)CCCN(CCC(=O)OCCCSSCCCCCCC)CCC(=O)OCCCSSCCCCCCC)CCC(=O)OCCCSSCCCCCCC] | 66 |
| 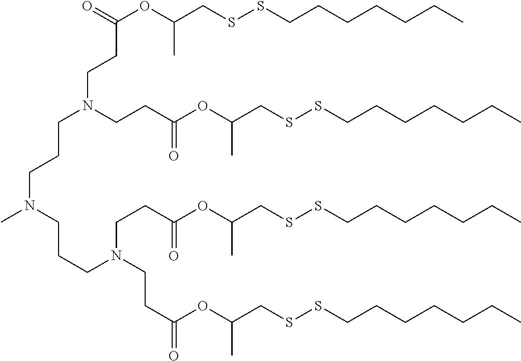 [SMILES: CCCCCCCSSCC(C)OC(=O)CCN(CCCN(C)CCCN(CCC(=O)OC(C)CSSCCCCCCC)CCC(=O)OC(C)CSSCCCCCCC)CCC(=O)OC(C)CSSCCCCCCC] | 67 |
| 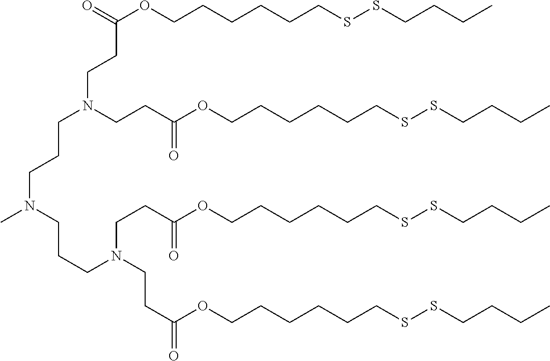 [SMILES: CCCCSSCCCCCCOC(=O)CCN(CCCN(C)CCCN(CCC(=O)OCCCCCCSSCCCC)CCC(=O)OCCCCCCSSCCCC)CCC(=O)OCCCCCCSSCCCC] | 68 |
| 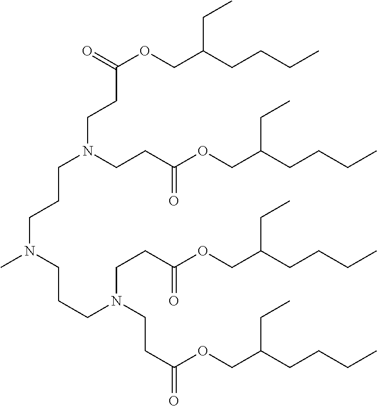 [SMILES: CCCCC(CC)COC(=O)CCN(CCCN(C)CCCN(CCC(=O)OCC(CC)CCCC)CCC(=O)OCC(CC)CCCC)CCC(=O)OCC(CC)CCCC] | 69 |
| 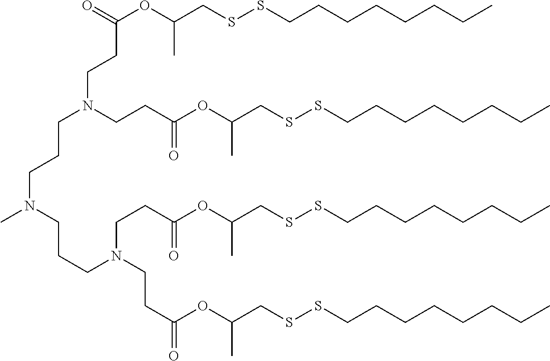 [SMILES: CCCCCCCCSSCC(C)OC(=O)CCN(CCCN(C)CCCN(CCC(=O)OC(C)CSSCCCCCCCC)CCC(=O)OC(C)CSSCCCCCCCC)CCC(=O)OC(C)CSSCCCCCCCC] | 70 |
| 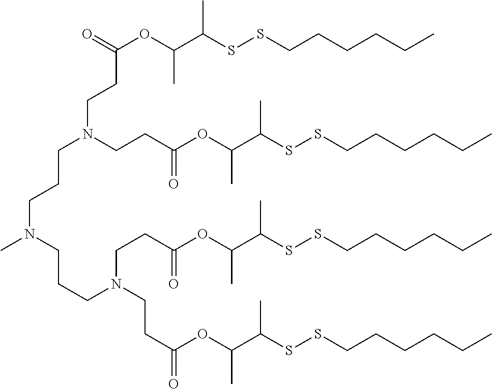 [SMILES: CCCCCCSSC(C)C(C)OC(=O)CCN(CCCN(C)CCCN(CCC(=O)OC(C)C(C)SSCCCCCC)CCC(=O)OC(C)C(C)SSCCCCCC)CCC(=O)OC(C)C(C)SSCCCCCC] | 71 |
| 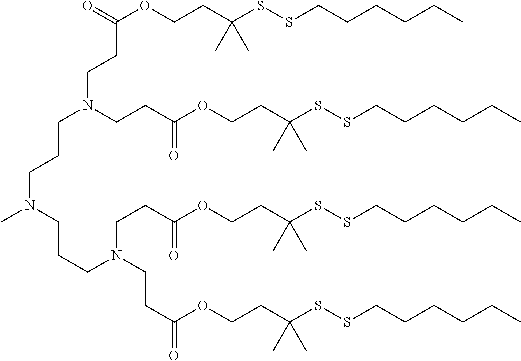 [SMILES: CCCCCCSSC(C)(C)CCOC(=O)CCN(CCCN(C)CCCN(CCC(=O)OCCC(C)(C)SSCCCCCC)CCC(=O)OCCC(C)(C)SSCCCCCC)CCC(=O)OCCC(C)(C)SSCCCCCC] | 72 |
| 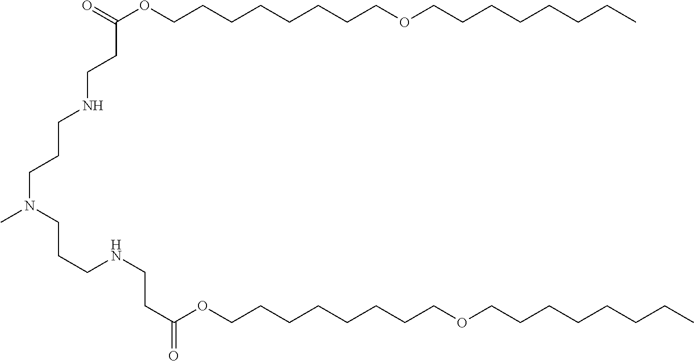 [SMILES: CCCCCCCCOCCCCCCCCOC(=O)CCNCCCN(C)CCCNCCC(=O)OCCCCCCCCOCCCCCCCC] | 73 |
| 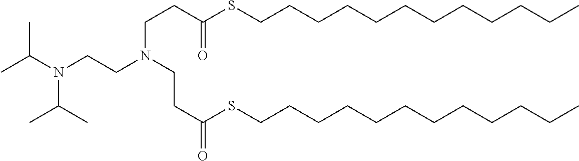 [SMILES: CCCCCCCCCCCCSC(=O)CCN(CCC(=O)SCCCCCCCCCCCC)CCN(C(C)C)C(C)C] | 74 |
| 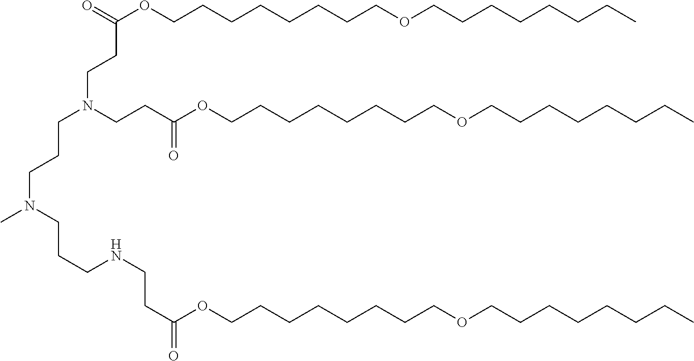 [SMILES: CCCCCCCCOCCCCCCCCOC(=O)CCNCCCN(C)CCCN(CCC(=O)OCCCCCCCCOCCCCCCCC)CCC(=O)OCCCCCCCCOCCCCCCCC] | 75 |
| 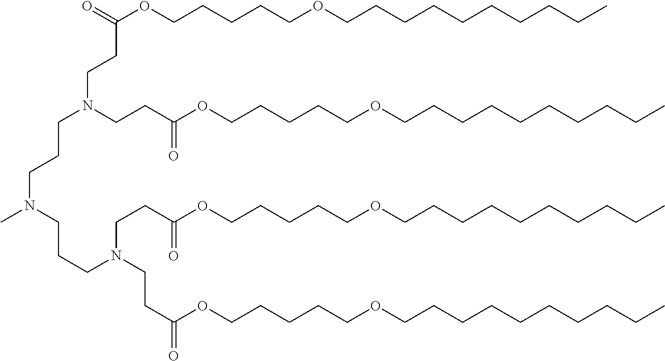 [SMILES: CCCCCCCCCCOCCCCCOC(=O)CCN(CCCN(C)CCCN(CCC(=O)OCCCCCOCCCCCCCCCC)CCC(=O)OCCCCCOCCCCCCCCCC)CCC(=O)OCCCCCOCCCCCCCCCC] | 77 |
| 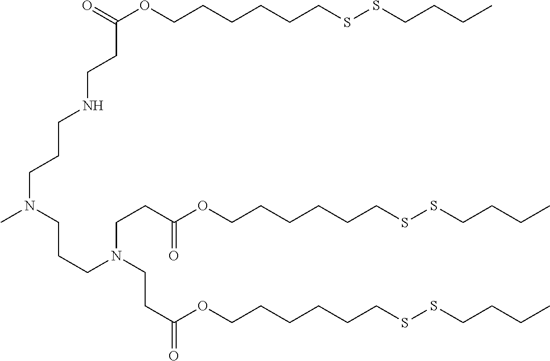 [SMILES: CCCCSSCCCCCCOC(=O)CCNCCCN(C)CCCN(CCC(=O)OCCCCCCSSCCCC)CCC(=O)OCCCCCCSSCCCC] | 78 |
| 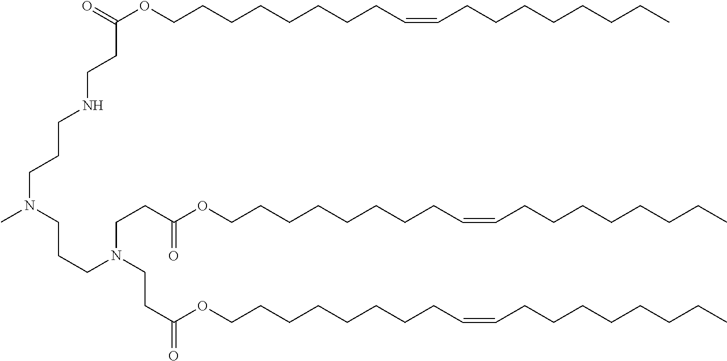 [SMILES: CCCCCCCC/C=C\CCCCCCCCOC(=O)CCNCCCN(C)CCCN(CCC(=O)OCCCCCCCC/C=C\CCCCCCCC)CCC(=O)OCCCCCCCC/C=C\CCCCCCCC] | 79 |
| 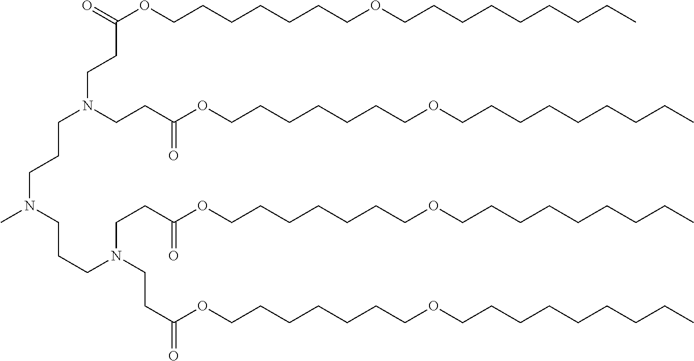 [SMILES: CCCCCCCCCOCCCCCCCOC(=O)CCN(CCCN(C)CCCN(CCC(=O)OCCCCCCCOCCCCCCCCC)CCC(=O)OCCCCCCCOCCCCCCCCC)CCC(=O)OCCCCCCCOCCCCCCCCC] | 80 |
| 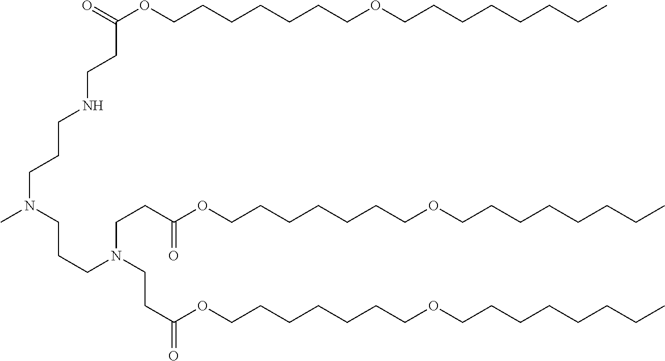 [SMILES: CCCCCCCCOCCCCCCCOC(=O)CCNCCCN(C)CCCN(CCC(=O)OCCCCCCCOCCCCCCCC)CCC(=O)OCCCCCCCOCCCCCCCC] | 81 |
| 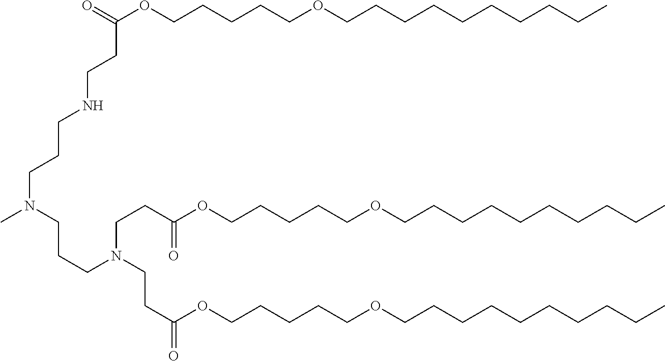 [SMILES: CCCCCCCCCCOCCCCCOC(=O)CCNCCCN(C)CCCN(CCC(=O)OCCCCCOCCCCCCCCCC)CCC(=O)OCCCCCOCCCCCCCCCC] | 82 |
| 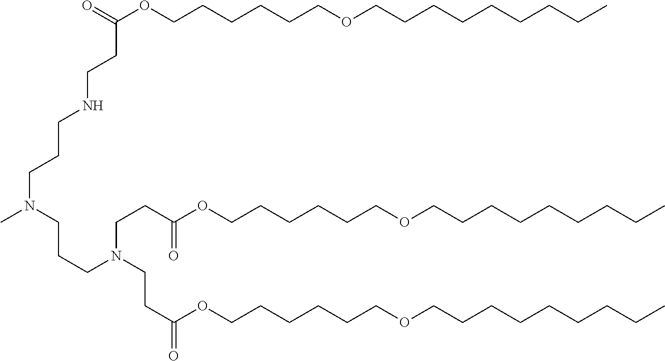 [SMILES: CCCCCCCCCOCCCCCCOC(=O)CCNCCCN(C)CCCN(CCC(=O)OCCCCCCOCCCCCCCCC)CCC(=O)OCCCCCCOCCCCCCCCC] | 83 |
| 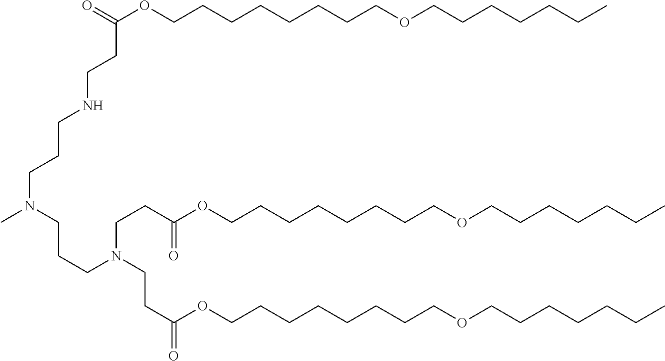 [SMILES: CCCCCCCOCCCCCCCCOC(=O)CCNCCCN(C)CCCN(CCC(=O)OCCCCCCCCOCCCCCCC)CCC(=O)OCCCCCCCCOCCCCCCC] | 84 |
| 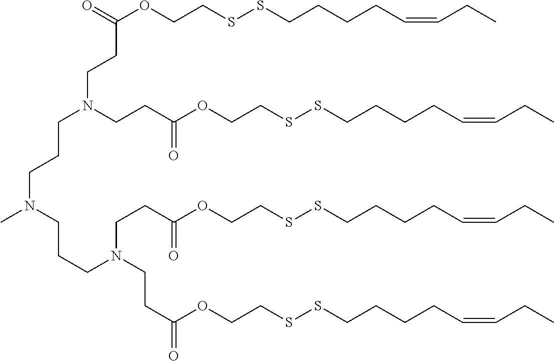 [SMILES: CC/C=C\CCCCSSCCOC(=O)CCN(CCCN(C)CCCN(CCC(=O)OCCSSCCCC/C=C\CC)CCC(=O)OCCSSCCCC/C=C\CC)CCC(=O)OCCSSCCCC/C=C\CC] | 85A |
| 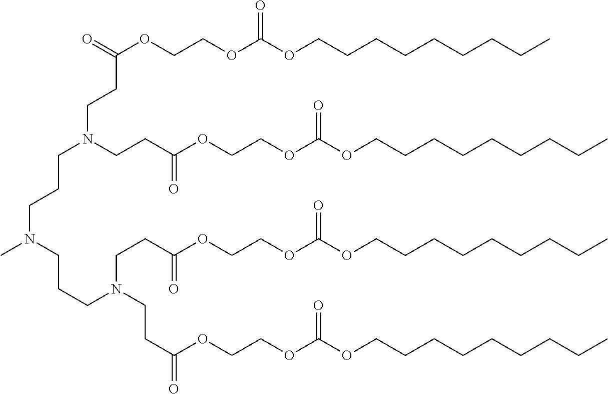 [SMILES: CCCCCCCCCOC(=O)OCCOC(=O)CCN(CCCN(C)CCCN(CCC(=O)OCCOC(=O)OCCCCCCCCC)CCC(=O)OCCOC(=O)OCCCCCCCCC)CCC(=O)OCCOC(=O)OCCCCCCCCC] | 85B |
| 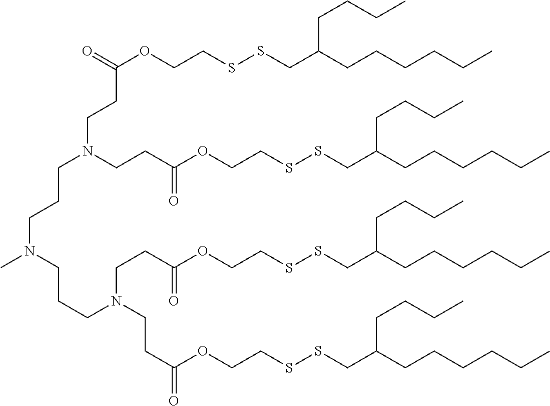 [SMILES: CCCCCCC(CCCC)CSSCCOC(=O)CCN(CCCN(C)CCCN(CCC(=O)OCCSSCC(CCCC)CCCCCC)CCC(=O)OCCSSCC(CCCC)CCCCCC)CCC(=O)OCCSSCC(CCCC)CCCCCC] | 86A |
| 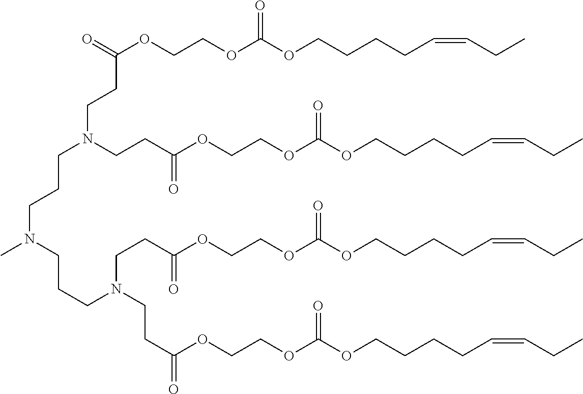 [SMILES: CC/C=C\CCCCOC(=O)OCCOC(=O)CCN(CCCN(C)CCCN(CCC(=O)OCCOC(=O)OCCCC/C=C\CC)CCC(=O)OCCOC(=O)OCCCC/C=C\CC)CCC(=O)OCCOC(=O)OCCCC/C=C\CC] | 86B |
| 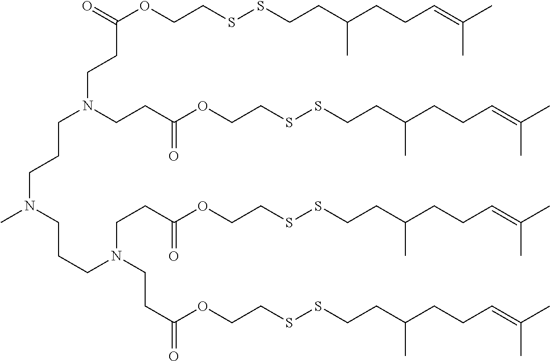 [SMILES: CC(C)=CCCC(C)CCSSCCOC(=O)CCN(CCCN(C)CCCN(CCC(=O)OCCSSCCC(C)CCC=C(C)C)CCC(=O)OCCSSCCC(C)CCC=C(C)C)CCC(=O)OCCSSCCC(C)CCC=C(C)C] | 87A |
| 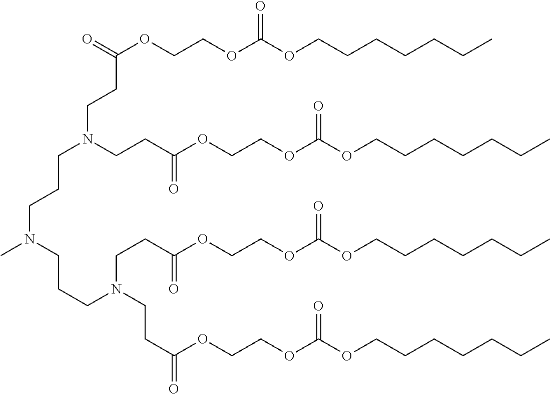 [SMILES: CCCCCCCOC(=O)OCCOC(=O)CCN(CCCN(C)CCCN(CCC(=O)OCCOC(=O)OCCCCCCC)CCC(=O)OCCOC(=O)OCCCCCCC)CCC(=O)OCCOC(=O)OCCCCCCC] | 87B |
| 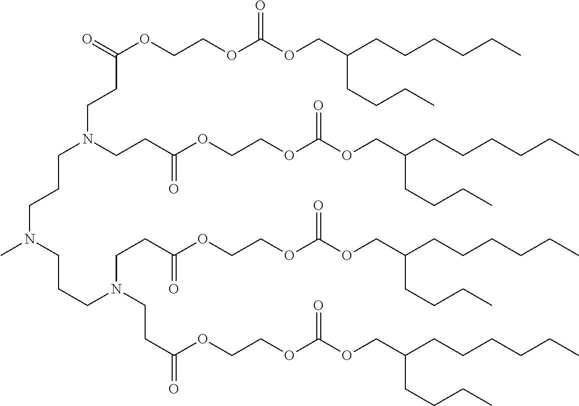 [SMILES: CCCCCCC(CCCC)COC(=O)OCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] | 88 |
| 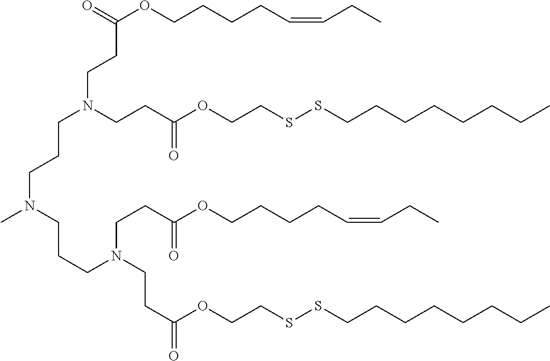 [SMILES: CC/C=C\CCCCOC(=O)CCN(CCCN(C)CCCN(CCC(=O)OCCCC/C=C\CC)CCC(=O)OCCSSCCCCCCCC)CCC(=O)OCCSSCCCCCCCC] | 89 |
| 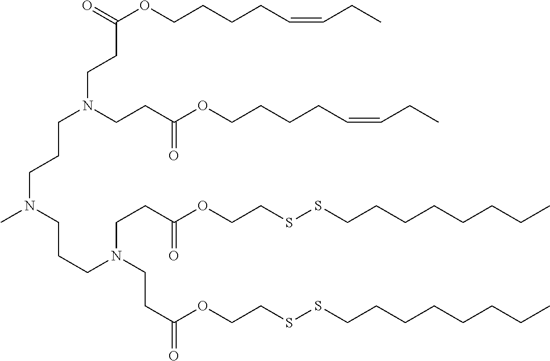 [SMILES: CC/C=C\CCCCOC(=O)CCN(CCCN(C)CCCN(CCC(=O)OCCSSCCCCCCCC)CCC(=O)OCCSSCCCCCCCC)CCC(=O)OCCCC/C=C\CC] | 90 |
| 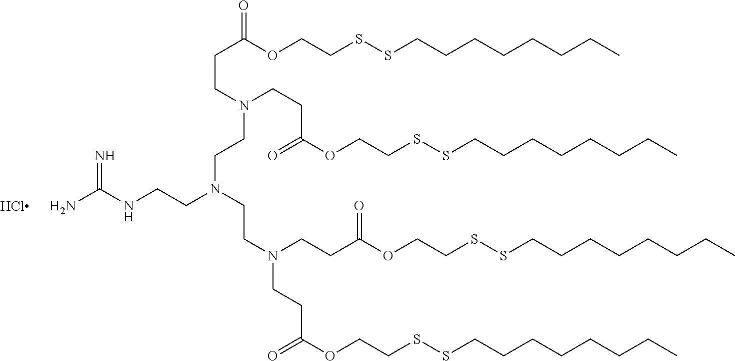 [SMILES: CCCCCCCCSSCCOC(=O)CCN(CCC(=O)OCCSSCCCCCCCC)CCN(CCNC(=N)N)CCN(CCC(=O)OCCSSCCCCCCCC)CCC(=O)OCCSSCCCCCCCC] | 91 |
| 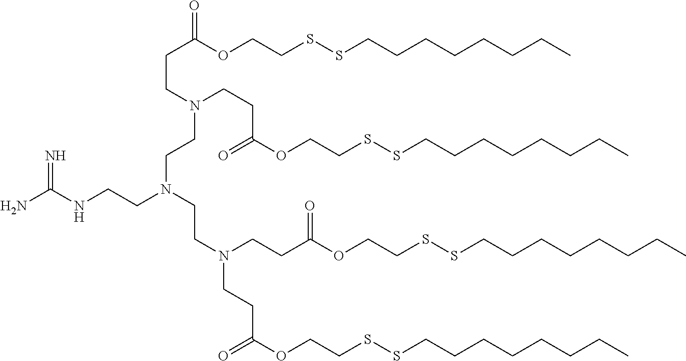 [SMILES: CCCCCCCCSSCCOC(=O)CCN(CCC(=O)OCCSSCCCCCCCC)CCN(CCNC(=N)N)CCN(CCC(=O)OCCSSCCCCCCCC)CCC(=O)OCCSSCCCCCCCC] | 92 |
| 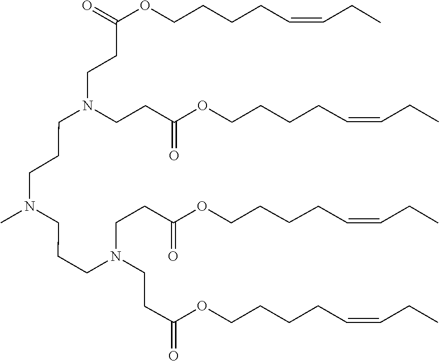 [SMILES: CC/C=C\CCCCOC(=O)CCN(CCCN(C)CCCN(CCC(=O)OCCCC/C=C\CC)CCC(=O)OCCCC/C=C\CC)CCC(=O)OCCCC/C=C\CC] | 93 |
| 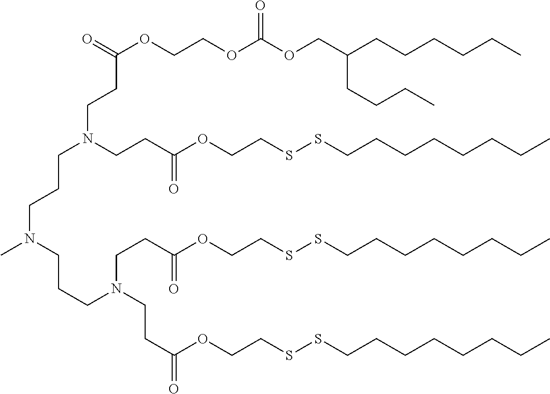 [SMILES: CCCCCCCCSSCCOC(=O)CCN(CCCN(C)CCCN(CCC(=O)OCCSSCCCCCCCC)CCC(=O)OCCSSCCCCCCCC)CCC(=O)OCCOC(=O)OCC(CCCC)CCCCCC] | 94 |
| 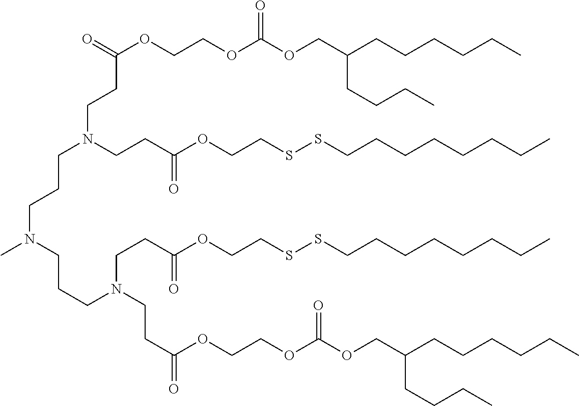 [SMILES: CCCCCCCCSSCCOC(=O)CCN(CCCN(C)CCCN(CCC(=O)OCCOC(=O)OCC(CCCC)CCCCCC)CCC(=O)OCCSSCCCCCCCC)CCC(=O)OCCOC(=O)OCC(CCCC)CCCCCC] | 95 |
| 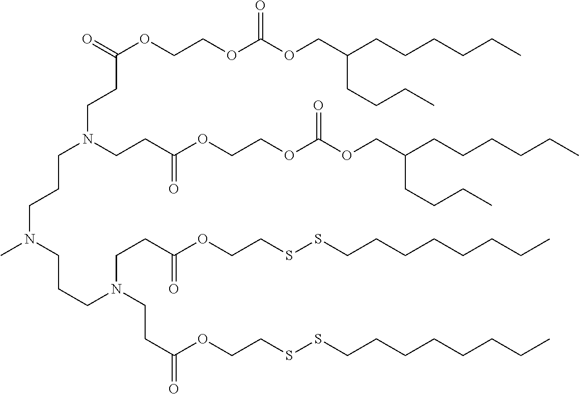 [SMILES: CCCCCCCCSSCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCSSCCCCCCCC] | 96 |
| 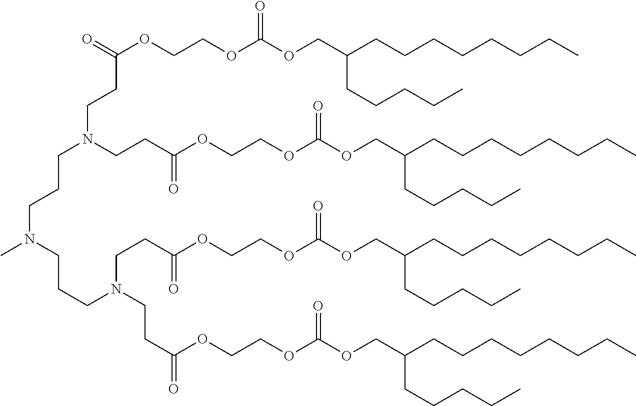 [SMILES: CCCCCCCCC(CCCCC)COC(=O)OCCOC(=O)CCN(CCCN(C)CCCN(CCC(=O)OCCOC(=O)OCC(CCCCC)CCCCCCCC)CCC(=O)OCCOC(=O)OCC(CCCCC)CCCCCCCC)CCC(=O)OCCOC(=O)OCC(CCCCC)CCCCCCCC] | 97 |
| 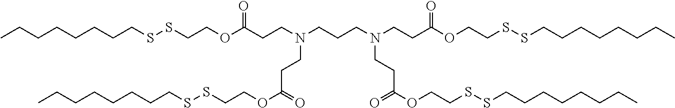 [SMILES: CCCCCCCCSSCCOC(=O)CCN(CCCN(CCC(=O)OCCSSCCCCCCCC)CCC(=O)OCCSSCCCCCCCC)CCC(=O)OCCSSCCCCCCCC] | 98 |
| 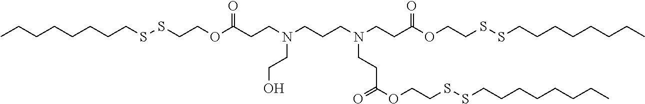 [SMILES: CCCCCCCCSSCCOC(=O)CCN(CCO)CCCN(CCC(=O)OCCSSCCCCCCCC)CCC(=O)OCCSSCCCCCCCC] | 99 |
| 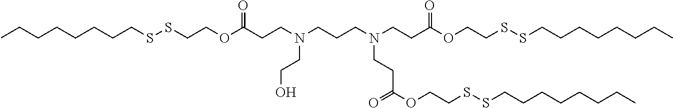 [SMILES: CCCCCCCCSSCCOC(=O)CCN(CCO)CCCN(CCC(=O)OCCSSCCCCCCCC)CCC(=O)OCCSSCCCCCCCC] | 100 |
| 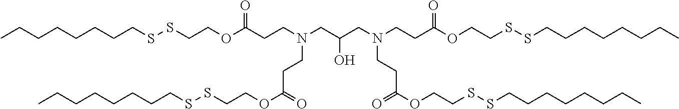 [SMILES: CCCCCCCCSSCCOC(=O)CCN(CCC(=O)OCCSSCCCCCCCC)CC(O)CN(CCC(=O)OCCSSCCCCCCCC)CCC(=O)OCCSSCCCCCCCC] | 101 |
| 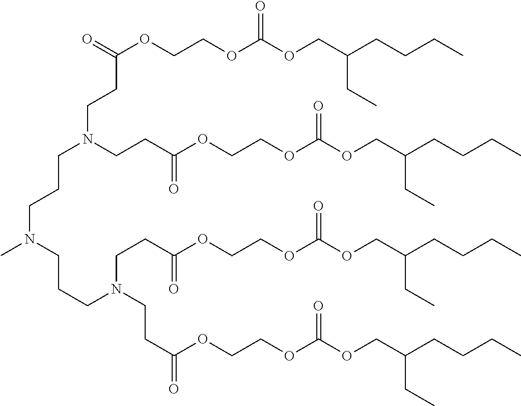 [SMILES: CCCCC(CC)COC(=O)OCCOC(=O)CCN(CCCN(C)CCCN(CCC(=O)OCCOC(=O)OCC(CC)CCCC)CCC(=O)OCCOC(=O)OCC(CC)CCCC)CCC(=O)OCCOC(=O)OCC(CC)CCCC] | 102 |
| 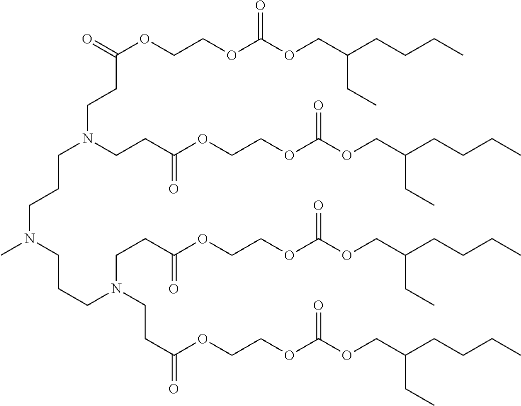 [SMILES: CCCCC(CC)COC(=O)OCCOC(=O)CCN(CCCN(C)CCCN(CCC(=O)OCCOC(=O)OCC(CC)CCCC)CCC(=O)OCCOC(=O)OCC(CC)CCCC)CCC(=O)OCCOC(=O)OCC(CC)CCCC] | 103 |
| 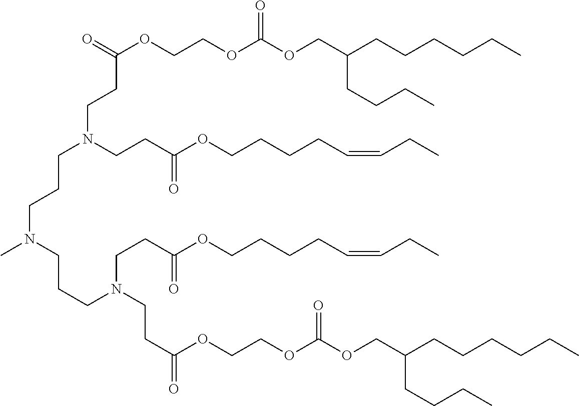 [SMILES: CC/C=C\CCCCOC(=O)CCN(CCCN(C)CCCN(CCC(=O)OCCCC/C=C\CC)CCC(=O)OCCOC(=O)OCC(CCCC)CCCCCC)CCC(=O)OCCOC(=O)OCC(CCCC)CCCCCC] | 104 |
| 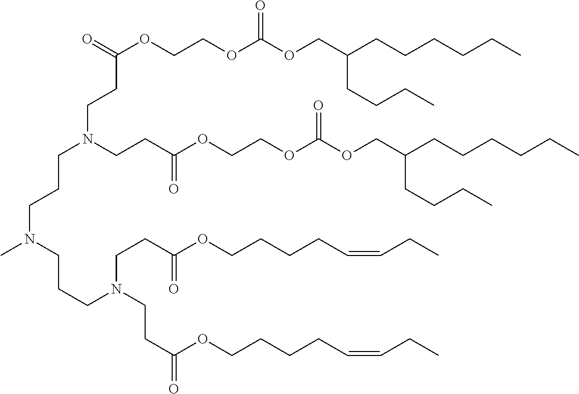 [SMILES: CC/C=C\CCCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCCC/C=C\CC] | 105 |
| 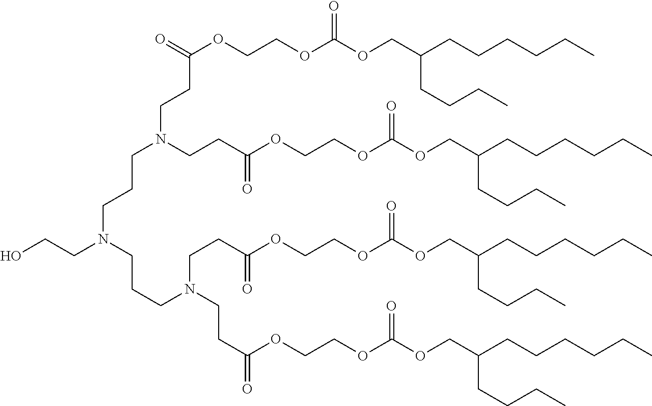 [SMILES: CCCCCCC(CCCC)COC(=O)OCCOC(=O)CCN(CCCN(CCO)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC] | 106 |

or a pharmaceutically acceptable salt of any of the foregoing.

In some embodiments, the lipidoid is:

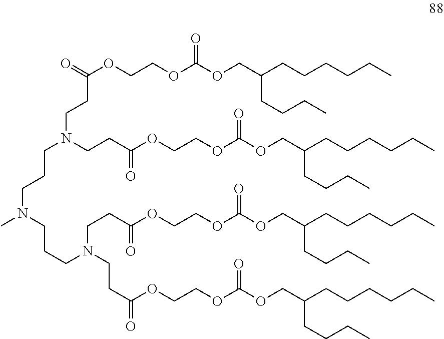 [SMILES: CCCCCCC(CCCC)COC(=O)OCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC]

or a pharmaceutically acceptable salt thereof.

In some embodiments, the composition provided herein further comprises a steroid. In some embodiments, the steroid comprises a cholesterol or a cholesterol derivative. In some embodiments, the composition provided herein further comprises a helper lipid. In some embodiments, the helper lipid comprises 1,2-dioleoyl-sn-glycero-3-phosphoethanolamine (DOPE) or 1,2-dioleoyl-sn-glycero-3-phosphocholine (DOPC). In some embodiments, the composition provided herein further comprises a polymer conjugated lipid. In some embodiments, the polymer conjugated lipid comprises 1,2-distearoyl-sn-glycero-3-phosphoethanolamine-N-[methoxy(polyethylene) (DSPE-PEG2k) or 1,2-dimyristoyl-rac-glycero-3-methoxypolyethylene glycol-2000 (DMG-PEG2k). In some embodiments, the lipid composition further comprises a modification comprising a cell-penetrating peptide. In some cases, the cell-penetrating peptide is transactivator of transcription (TAT).

In some embodiments, the lipid composition comprises a lipidoid disclosed in this application, a steroid, a helper lipid, and a polymer conjugated lipid.

In some embodiments, the lipidoid is present in the lipid composition at a weight percentage from about 10% to about 20%, about 10% to about 30%, about 10% to about 40%, about 10% to about 50%, about 10% to about 60%, about 10% to about 80%, about 10% to about 90%, from about 20% to about 30%, from about 20% to about 30%, from about 20% to about 40%, from about 20% to about 50%, from about 20% to about 60%, from about 20% to about 70%, from about 20% to about 80%, from about 20% to about 90%, from about 30% to about 40%, from about 30% to about 50%, from about 30% to about 60%, from about 30% to about 70%, from about 30% to about 80%, from about 30% to about 90%, from about 40% to about 50%, from about 40% to about 60%, from about 40% to about 70%, from about 40% to about 80%, from about 40% to about 90%, from about 50% to about 60%, from about 50% to about 70%, from about 50% to about 80%, from about 50% to about 90%, from about 60% to about 70%, from about 60% to about 80%, from about 60% to about 90%, from about 70% to about 80%, from about 70% to about 90%, or from about 80% to about 90%.

In some embodiments, the helper lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the steroid is present in the lipid composition at a weight percentage from about 10% to about 20%, from about 10% to about 30%, from about 10% to about 40%, from about 20% to about 30%, from about 20% to about 40%, or from about 30% to about 40%.

In some embodiments, the polymer conjugated lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the weight ratio of the lipidoid/steroid/helper lipid/polymer conjugated lipid is about 14/4/1/1, about 15/4/1/1, about 16/4/1/1, about 17/4/1/1, about 18/4/1/1, about 19/4/1/1, about 20/4/1/1, about 14/4/2/1, about 15/4/2/1, about 16/4/2/1, about 16/4/2.7/1, about 16/4/2/2, about 16/4/2/1, about 16/4/3/1, about 16/4/3/2, about 16.8/4/2/1, about 17/4/2/1, about 18/4/2/1, about 19/4/2/1, or about 20/4/2/1. In some embodiments, the weight ratio of the lipidoid/steroid/helper lipid/polymer conjugated lipid is about 16/7.44/3.35/1.86, about 16/7.5/3.5/1.8, about 16/8/3.5/1.8, about 16/8/3.5/2.4, about 16/8/4/1.5, or about 16/8/5/3.

In some embodiments, the lipid composition comprises a lipidoid disclosed in this application, a steroid and a helper lipid. In some embodiments, the lipidoid is present in the lipid composition at a weight percentage from about 30% to about 90%. In some embodiments, the helper lipid is present in the lipid composition at a weight percentage from about 5% to about 40%. In some embodiments, the steroid is present in the lipid composition at a weight percentage from about 5% to about 40%. In some embodiments, the weight ratio of the lipidoid/steroid/helper lipid is about 1/1/1, 2/1/1, about 3/1/1, about 4/1/1, about 5/1/1, about 6/1/1, about 2/2/1, about 3/2/1, about 4/2/1, about 5/2/1, or about 6/2/1.

In some embodiments, the lipid composition further comprises an excipient. The excipient can comprise EC-16, (2-hydroxypropyl)-β-cyclodextrin ((HP-β-CD), stearic acid, Perfluoroundecanoic, Saponin, Mannitol, Borneol, Amikacin-EC16, Kanamycin-EC16, Neomycin-EC16, or Bile salts. In some embodiments, the excipient is present in the composition at a weight percentage from about 5% to about 60%. In some embodiments, the excipient is present in the composition at a weight percentage from about 1% to about 70%, from about 5% to about 60%, from about 5% to about 50%, from about 5% to about 40%, from about 5% to about 30%, from about 10% to about 50%, from about 10% to about 40%, from about 10% to about 30%, or from about 10% to about 20%.

In some embodiments, the pharmaceutical agent comprises a polynucleotide, an oligonucleotide, a polypeptide, an oligopeptide, a small molecule compound, or any combination thereof. In some embodiments, the polynucleotide is a messenger ribonucleic acid (mRNA).

In some embodiments, the pharmaceutical agent comprises a polynucleotide that encodes or is configured to regulate a target gene or a gene product thereof.

In some embodiments, the pharmaceutical agent comprises (a) a gene modulating moiety configured to specifically bind at least a portion of a target gene or a gene product thereof; or (b) a polynucleotide that encodes the gene modulating moiety of (a).

In some embodiments, the gene modulating moiety comprises a guide nucleic acid configured to complex with at least a portion of the target gene or the gene product thereof, or a polynucleotide sequence that encodes the guide nucleic acid.

In some embodiments, the gene modulating moiety comprises a heterologous endonuclease or a polynucleotide comprising a sequence that encodes the heterologous endonuclease. In some embodiments, the heterologous endonuclease comprises a clustered regularly interspaced short palindromic repeats (CRISPR)-associated (Cas) nuclease. In some embodiments, heterologous endonuclease is an adenine base editor (e.g., ABE) comprising an adenine base editor 8e (e.g., ABE8e). In some embodiments, the polynucleotide comprising a sequence that encodes the heterologous endonuclease.

In some embodiments, pharmaceutical agent is assembled in the lipid composition at a weight ratio of the pharmaceutical agent/lipid composition of from about 1:200 to about 1:100, from about 1:200 to about 1:50, from about 1:200 to about 1:40, from about 1:200 to about 1:30, from about 1:200 to about 1:20, from about 1:200 to about 1:10, from about 1:200 to about 1:5, from about 1:200 to about 1:1, from about 1:100 to about 1:50, from about 1:100 to about 1:40, from about 1:100 to about 1:25, from about 1:100 to about 1:20, from about 1:100 to about 1:15, from about 1:100 to about 1:10, from about 1:100 to about 1:5 or from about 1:100 to about 1:1.

In some embodiments, the target gene or the gene product thereof is specific to or primarily found in a target organ (e.g., liver) or a target cell of a subject.

In some embodiments, the target gene or the gene product thereof is associated with a disease or disorder of the target organ or the target cell.

In some embodiments, the gene modulating moiety is configured to provide a modified expression profile of the target gene or the gene product thereof in a target organ (e.g., liver) or a target cell of a subject.

In some embodiments, the composition is formulated for systemic or local administration. In some embodiments, the composition is formulated for intravenous administration. In some embodiments, the composition is formulated for intramuscular administration.

### Additional Lipids

In some embodiments, the lipid composition further comprises an additional lipid comprising a steroid or a steroid derivative, a PEG lipid, and a helper lipid (e.g., phospholipids or other zwitterionic lipids).

In some embodiments, the lipid composition further comprises a helper lipid. In some embodiments, the helper lipid comprises a lipid that contributes to the stability or delivery efficiency of the lipid compositions. In some embodiments, the helper lipid comprises a zwitterionic lipid. In some embodiments, the helper lipid comprises a phospholipid. In some embodiments, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge. In some embodiments of the lipid compositions, the phospholipid is not an ethylphosphocholine. In some embodiments, the helper lipid can comprise 1,2-dioleoyl-sn-glycero-3-phosphoethanolamine (DOPE) or 1,2-dioleoyl-sn-glycero-3-phosphocholine (DOPC).

In some embodiments, the compositions may further comprise a molar percentage of the phospholipid to the total lipid composition from about 5 to about 30.

In some embodiments, the helper lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the lipid composition comprises the phospholipid at a molar percentage from about 8% to about 23%. In some embodiments, the lipid composition comprises the phospholipid at a molar percentage from about 10% to about 20%. In some embodiments, the lipid composition comprises the phospholipid at a molar percentage from about 15% to about 20%. In some embodiments, the lipid composition comprises the phospholipid at a molar percentage from about 8% to about 15%. In some embodiments, the lipid composition comprises the phospholipid at a molar percentage from about 10% to about 15%. In some embodiments, the lipid composition comprises the phospholipid at a molar percentage from about 12% to about 18%. In some embodiments, the lipid composition comprises the phospholipid at a molar percentage of at least about 8%, at least about 10%, at least about 12%, at least about 15%, at least about 18%, at least about 20%, or at least about 23%. In some embodiments, the lipid composition comprises the phospholipid at a molar percentage of at most about 8%, at most about 10%, at most about 12%, at most about 15%, at most about 18%, at most about 20%, or at most about 23%.

In some embodiments, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

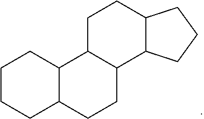 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

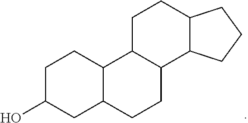 [SMILES: OC1CCC2C(CCC3C4CCCC4CCC23)C1]

In some embodiments, the steroid or steroid derivative is a cholestane or cholestane derivative. In a cholestane, the ring structure is further defined by the formula:

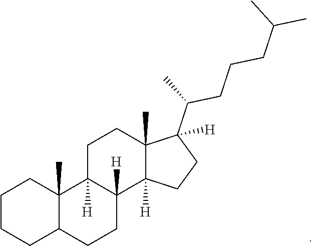

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments, the compositions may further comprise a molar percentage of the steroid to the total lipid composition from about 20 to about 60. In some embodiments, the steroid is present in the lipid composition at a weight percentage from about 10% to about 20%, from about 10% to about 30%, from about 10% to about 40%, from about 20% to about 30%, from about 20% to about 40%, or from about 30% to about 40%.

In some embodiments, the lipid composition comprises the steroid or steroid derivative at a molar percentage from about 15% to about 46%. In some embodiments, the lipid composition comprises the steroid or steroid derivative at a molar percentage from about 20% to about 40%. In some embodiments, the lipid composition comprises the steroid or steroid derivative at a molar percentage from about 25% to about 35%. In some embodiments, the lipid composition comprises the steroid or steroid derivative at a molar percentage from about 30% to about 40%. In some embodiments, the lipid composition comprises the steroid or steroid derivative at a molar percentage from about 20% to about 30%. In some embodiments, the lipid composition comprises the steroid or steroid derivative at a molar percentage of at least about 15%, at least about 20%, at least about 25%, at least about 30%, at least about 35%, at least about 40%, at least about 45%, or at least about 46%. In some embodiments, the lipid composition comprises the steroid or steroid derivative at a molar percentage of at most about 15%, at most about 20%, at most about 25%, at most about 30%, at most about 35%, at most about 40%, at most about 45%, or at most about 46%.

In some embodiments, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments, the PEG lipid has a structural formula:

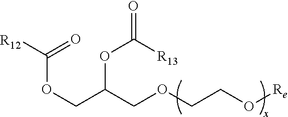

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments, the PEG lipid has a structural formula:

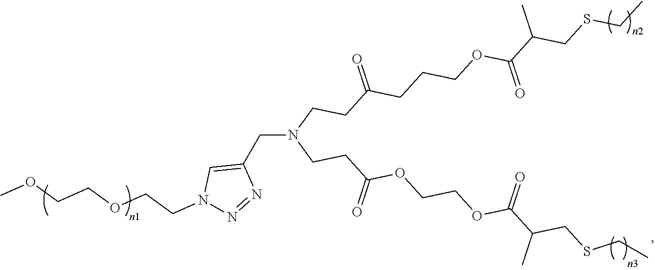

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k).

In some embodiments, the lipid composition comprises the polymer-conjugated lipid at a molar percentage from about 0.5% to about 20%. In some embodiments, the lipid composition comprises the polymer-conjugated lipid at a molar percentage from about 1% to about 8%. In some embodiments, the lipid composition comprises the polymer-conjugated lipid at a molar percentage from about 2% to about 7%. In some embodiments, the lipid composition comprises the polymer-conjugated lipid at a molar percentage from about 3% to about 5%. In some embodiments, the lipid composition comprises the polymer-conjugated lipid at a molar percentage from about 5% to about 10%. In some embodiments, the lipid composition comprises the polymer-conjugated lipid at a molar percentage of at least about 0.5%, at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, or at least about 10%. In some embodiments, the lipid composition comprises the polymer-conjugated lipid at a molar percentage of at most about 0.5%, at most about 1%, at most about 1.5%, at most about 2%, at most about 2.5%, at most about 3%, at most about 3.5%, at most about 4%, at most about 4.5%, at most about 5%, at most about 5.5%, at most about 6%, at most about 6.5%, at most about 7%, at most about 7.5%, at most about 8%, at most about 8.5%, at most about 9%, at most about 9.5%, at most about 10%, at most about 15%, or at most 20%.

In some embodiments, the polymer conjugated lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the lipidoid is present in the lipid composition at a molar percentage of no more than about 60%, no more than about 50%, or no more than about 40%.

In some embodiments, the steroid or steroid derivative is present in the lipid composition at a molar percentage of no more than about 50%.

In some embodiments, the polymer-conjugated lipid is present in the lipid composition at a molar percentage of no more than about 10%.

In some embodiments, the lipid composition further comprises a phospholipid.

In some embodiments, the lipid composition further comprises a phosphoethanolamine lipid or a phosphocholine lipid. In some embodiments, the phospholipid is present in the lipid composition at a molar percentage of no more than about 30%.

In some embodiments, the pharmaceutical agent is a polynucleotide, an oligonucleotide, a polypeptide, an oligopeptide, a small molecule compound, or any combination thereof regulate a target gene or a gene product thereof.

In some embodiments, the pharmaceutical agent is a polynucleotide that encodes or is configured to regulate a target gene or a gene product thereof.

In some embodiments, the pharmaceutical agent is a messenger ribonucleic acid (mRNA), an oligonucleotide, a polypeptide, an oligopeptide, a small molecule compound, or any combination thereof.

In some embodiments, the polypeptide is a protein.

In some embodiments, the polynucleotide is configured to up regulate or downregulate a target gene or a gene product thereof.

In some embodiments, the target gene or the gene product thereof is a target protein or a functional variant thereof or a target transcript.

In some embodiments, the polynucleotide is a messenger ribonucleic acid (mRNA) that encodes or is configured to upregulate or downregulate a target gene or a gene product thereof.

In some embodiments, the target gene or the gene product thereof is a target protein or a function variant thereof or a target transcript.

In some embodiments, the target transcript is in a target organ or a target cell.

In some embodiments, the pharmaceutical agent comprises: (a) a gene modulating moiety configured to specifically bind at least a portion of a target gene or a gene product thereof; or (b) a polynucleotide that encodes the gene modulating moiety of (a).

In some embodiments, the gene modulating moiety is a guide nucleic acid configured to complex with at least a portion of the target gene or the gene product thereof, or a polynucleotide sequence that encodes the guide nucleic acid.

In some embodiments, the target gene or the gene product is a target protein or a functional variant thereof or a target transcript.

In some embodiments, the polynucleotide is a messenger ribonucleic acid (mRNA)) that encodes the gene modulating moiety of (a).

In some embodiments, the gene modulating moiety is a heterologous endonuclease or a polynucleotide comprising a sequence that encodes the heterologous endonuclease.

In some embodiments, the heterologous endonuclease is a clustered regularly interspaced short palindromic repeats (CRSIPR)-associated (Cas) nuclease.

In some embodiments, heterologous endonuclease is an adenine base editor (e.g., ABE) comprising an adenine base editor 8e (e.g., ABE8e).

In some embodiments, the polynucleotide is a messenger ribonucleic acid (mRNA).

In some embodiments, the heterologous endonuclease is present in the gene modulating moiety at a mass or weight ratio to the guide nucleic acid of about 1:20 to about 20:1.

In some embodiments, the heterologous endonuclease is present in the gene modulating moiety at a mass or weight ratio to the guide nucleic acid of about 1:10 to about 10:1.

In some embodiments, the target gene or the gene product thereof is specific to or primarily found in a target organ or a target cell of a subject.

In some embodiments, the target gene or the gene product thereof is a target protein or a functional variant thereof or the target transcript.

In some embodiments, the target gene or the gene product thereof is associated with a disease or disorder of the target organ or the target cell.

In some embodiments, the gene modulating moiety is configured to provide a modified expression profile of the target gene or the gene product thereof in a target organ or a target cell of a subject.

In some embodiments, heterologous endonuclease is an adenine base editor (e.g., ABE) comprising an adenine base editor 8e (e.g., ABE8e).

In some embodiments, the target organ is liver. In some embodiments, the target cell is a liver cell.

In some embodiments, the composition is formulated for administration. In some embodiments, the composition is formulated for local administration. In some embodiments, the composition is formulated for systemic administration.

In some embodiments, the lipidoid or the pharmaceutically acceptable salt thereof, is present in the lipid composition at a molar percentage of about 20% to about 50%.

In some embodiments, the lipid composition comprises a steroid or steroid derivative at a molar percentage of about 10% to about 50%.

In some embodiments, the lipidoid or the pharmaceutically acceptable salt thereof, is present in the composition at a mass or weight ratio to the pharmaceutical agent of about 5:1 to about 100:1.

In some embodiments, the composition is for preferential delivery of the pharmaceutical agent to liver or a liver cell as compared to delivery to a non-liver organ or a non-liver cell.

In some embodiments, the pharmaceutical agent is delivered to a liver or a liver cell in a subject.

In some embodiments, the non-liver organ is a lung, heart, spleen or kidney, and wherein the non-liver cell is a lung cell, a heart cell, a spleen cell, or a kidney cell.

In some embodiments, the pharmaceutical agent encodes or is configured to regulate a target gene or a gene product thereof that is specific to or primarily found in the liver or the liver cell.

In some embodiments, the pharmaceutical agent encodes or is configured to up-regulate a target gene or a gene product thereof that is specific to or primarily found in the liver or the liver cell.

In some embodiments, the target gene is an angiopoietin-like 3 (ANGPTL3) gene.

In some embodiments, the pharmaceutical agent encodes or is configured to down-regulate a target gene or a gene product thereof that is specific to or primarily found in the liver or the liver cell.

In some embodiments, the target gene or gene product thereof is a target protein or a functional variant thereof or a target transcript.

In some embodiments, the pharmaceutical agent is configured to provide a modified expression profile of the target gene or the gene product thereof (e.g., the target protein or the functional variant thereof, or the target transcript) in the liver or the liver cell.

In some embodiments, the pharmaceutical agent is associated with a liver disease or disorder

### Payloads

LNPs of the present disclosure can comprise an ionizable lipid and a plurality of payloads comprising a pharmaceutical agents provided herein (e.g., polynucleotides and/or polypeptides). In some embodiments, the payload (e.g., pharmaceutical agent) is a polynucleotide provided herein (e.g., an mRNA and/or a short RNA). In some embodiments, the mRNA provided herein encode a polypeptide provided herein. In some embodiments, the LNPs comprise mRNA encoding a viral antigen (e.g., a spike protein). The LNPs comprising a spike mRNA (e.g., spike mRNA/LNPs) can be used for vaccinating against a SARS-CoV2 variant. In some embodiments, the SARS-CoV2 variant comprises Omicron BQ.1, XBB.1, BA.4, BA.5, BA.5.2.6, BF.7, BF.1.1, BA.2, BA.2.12.2, BA.1, BA.1.1, wildtype Omicron, Alpha, Beta, Gamma, Delta, Lambda, or Mu strain of SARS-CoV-2.

The LNPs comprising ionizable lipids can comprise an mRNA modified with regulatory sequences (e.g., untranslated regions, UTRs).). Regulatory sequences can modulate (e.g., enhance or reduce) the expression of peptides encoded by the mRNA. In some embodiments, the regulatory sequences can additionally modulate (e.g., improve or reduce) delivery of the mRNA formulated LNPs (e.g., mRNA/LNPs) to a target organ (e.g., liver, lymph nodes, or spleen) or a target cell (e.g., immune cells or hepatocyte). The untranslated regions (UTRs) can comprise 5′ UTRs and/or 3′ UTRs. In some cases, the 3′ UTRs are MmB-globin, ha-globin, C3, TIAM1, P450 2E1, AP3B1, MS10433, Apo A-II, POTEE, S0_M_T1012, YY2 TF, OXRlor WIPI2. In some cases, the 5′ UTRs are 70 nt (GG) or Ces1d. The mRNA/LNPs can comprise any one or more UTRs selected from the group MmB-globin, ha-globin, C3, TIAM1, P450 2E1, AP3B1, MS10433, Apo A-II, POTEE, S0_M_T1012, YY2 TF, OXR1, WIPI2, 70 nt (GG) and Ces1d. In some cases, the mRNA/LNPs comprise 5′ UTR/3′UTR combination of Ces1d and AP3B1. In some cases, the mRNA/LNPs comprise 5′ UTR/3′UTR combination of 70 nt (GG) and Apo A-II.

The LNPs comprising ionizable lipids can comprise an mRNA encoding a therapeutic protein. In some cases, the mRNA can encode a therapeutic protein for restoring a defective or deficient protein in a subject suffering from having a defective or deficient protein. Cigler-Najjar (CN) syndrome is a hereditary autosomal recessive disorder with an incidence of less than 1 case for 1 million people. It is caused by abnormalities in the gene coding for uridine diphosphoglucuronate glucuronosyltransferase (UGT1A1). UGT1A1 normally catalyzes the conjugation of bilirubin and glucuronic acid within hepatocytes. In CN syndrome, the malfunctioning of the process of bilirubin elimination from the bile mediated by the enzyme causes build-up of free bilirubin in the serum, which can become neurotoxic. Depending on the mutation of the gene, the severity of CN syndrome can vary from mild, with moderate increase in serum bilirubin rates, to severe, with high levels. In some embodiments, LNPs comprise mRNA encoding UGTLAL (e.g., Ug1a1 mRNA/LNPs) and can be used to treat Cigler-Najjar (CN) syndrome of a subject.

The LNPs comprising ionizable lipids can comprise more than one types of nucleic acids. In some embodiments, the LNPs comprise an mRNA encoding a protein for gene editing (e.g. an adenine base editor; a CRISPR-associated protein) and a short RNA (e.g., sgRNA). Angptl3 has been identified as a potential target of interest for treating dyslipidemia diseases. Angptl3 encodes for angiopoietin-like 3 (ANGPTL3), an enzyme that regulates plasma lipoprotein levels. ANGPTL3 is expressed by liver hepatocyte and is secreted into the bloodstream. Loss of function (LOF) variants of Angptl3 are naturally occurring in the human population with no known associated complications. Individuals with these mutations have an improved lipid profile, for example, reduced levels of plasma low density lipoprotein cholesterol (LDL-C) and triglycerides (TG), as well as protection against CAD. In some embodiments, the LNPs provided herein comprise mRNA encoding a protein for gene editing and a short RNA to modulate the Angptl3 gene. In some embodiments, the LNPs comprise mRNA encoding an adenine base editor 8e (e.g., ABE8e) and a corresponding sgRNA targeting the Angptl3 gene. In some embodiments, delivery of the mRNA/sgRNA cargo provided herein improves lipid profile (e.g., reduction in LDL-C and TG) of a subject affected by dyslipidemia or coronary artery disease.

### Methods

Provided herein are methods for delivering a payload (e.g., a pharmaceutical agent comprising a polynucleotide) to a target organ or a target cell in a subject in need thereof, where the method comprises administering the composition provided herein. In some cases, the methods comprise administering the lipids and compositions provided herein through systemic administration (e.g., intravenous injection). In some cases, the methods comprise administering the lipids and compositions provided herein through local administration (e.g., intramuscular injection).

One aspect of the present disclosure relates to methods for vaccinating against a SARS-CoV2 variant by administering the spike mRNA/LNPs of the present disclosure. In some embodiments, the SARS-CoV2 variant comprises Omicron BQ.1, XBB.1, BA.4, BA.5, BA.5.2.6, BF.7, BF.1.1, BA.2, BA.2.12.2, BA.1, BA. 1.1, wildtype Omicron, Alpha, Beta, Gamma, Delta, Lambda, or Mu strain of SARS-CoV-2. In some embodiments, the spike mRNA/LNPs further comprise a regulatory sequence (e.g., UTR). In some embodiments, administering the spike mRNA/LNPs provided herein to a subject lead to high anti-spike IgG titer, high T-cell response, and/or reduced viral load compared to a spike mRNA/LNP not comprising the ionizable lipids of the present disclosure.

Another aspect of the present disclosure relates to methods for improved mRNA/LNP delivery to a target organ (e.g., liver, lymph nodes, or spleen) or a target cell (e.g., immune cells or hepatocyte) by administering a mRNA/LNP comprising an ionizable lipid and a regulatory sequence. In some embodiments, the regulatory sequence comprises 5′ UTRs and/or 3′ UTRs. In some cases, the 3′ UTRs are Mmβ-globin, ha-globin, C3, TIAM1, P450 2E1, AP3B1, MS10433, Apo A-II, POTEE, S0_M_T1012, YY2 TF, OXR1 or WIPI2. In some cases, the 5′ UTRs are 70 nt (GG) or Ces1d. The mRNA/LNPs can comprise any one or more UTRs selected from the group Mmβ-globin, ha-globin, C3, TIAM1, P450 2E1, AP3B1, MS10433, Apo A-II, POTEE, S0_M_T1012, YY2 TF, OXR1, WIPI2, 70 nt (GG) and Ces1d. In some cases, the mRNA/LNPs comprise 5′ UTR/3′UTR combination of Ces1d and AP3B1. In some cases, the mRNA/LNPs comprise 5′ UTR/3′UTR combination of 70 nt (GG) and Apo A-II.

Also provided herein are methods for treating Cigler-Najjar (CN) syndrome by administering the LNPs of the present disclosure, wherein the LNPs comprise an mRNA encoding a UGTLAL protein (e.g., Ug1a1 mRNA/LNPs).

Another aspect of the disclosure relates to methods of treating a disease by administering the mRNA/LNPs provided herein to a subject. In some embodiments, the disease is a dyslipidemia disease or a coronary artery disease. In some embodiments, the method comprises administering LNPs formulated with an mRNA encoding a protein for gene editing (e.g., ABE, ABE8e, a Cas) and a short RNA (e.g., sgRNA) targeting the Angptl3 gene to modulate the Angptl3 gene. In some embodiments, administering the mRNA/sgRNA to a subject improves lipid profile (e.g., reduction in LDL-C and TG) of a subject affected by dyslipidemia or coronary artery disease.

Provided herein are methods for preferential delivery of a pharmaceutical agent (e.g., a polynucleotide, a mRNA, and/or a sgRNA) to liver or a liver cell in a subject in need thereof, the method comprising administering the composition provided herein, thereby providing a greater amount, e.g., at least about 2-fold greater amount, at least about 3-fold greater amount, at least about 3-fold greater amount, at least about 4-fold greater amount, at least about 5-fold greater amount or at least about 10-fold greater amount, expression or activity of the pharmaceutical agent in the liver or the liver cell of the subject as compared to that achieved in a non-liver organ or a non-liver cell in the subject. In some embodiments, the non-liver organ is a lung, heart, spleen, or kidney, and wherein the non-liver cell is a lung cell, a heart cell, a spleen cell, or a kidney cell.

Provided herein are methods for preferential delivery of a pharmaceutical agent (e.g., a polynucleotide, a mRNA, and/or a sgRNA) to liver or a liver cell in a subject in need thereof, the method comprising administering the composition provided herein, thereby providing a greater amount, e.g., at least about a 2-fold greater amount, at least about 3-fold greater amount, at least about 3-fold greater amount, at least about 4-fold greater amount, at least about 5-fold greater amount or at least about 10-fold greater amount expression or activity of the pharmaceutical agent in the liver or the liver cell of the subject as compared to that achieved with a corresponding reference lipid composition comprising a corresponding reference lipidoid.

In some embodiments, the method provided herein modulates a greater amount (e.g., at least about 2-fold greater amount) or activity of a target gene or a gene product thereof in the liver or the liver cell of the subject as compared to that achieved with a corresponding reference lipid composition comprising a corresponding reference lipidoid.

In some embodiments, the corresponding reference lipidoid has an amide-containing tail.

In some embodiments, the method provides a modified expression profile of the target gene or the gene product thereof in the liver or the liver cell of the subject. In some embodiment, the target gene is a Angptl3 gene. In some embodiment, the target gene is a Ug1a1 gene.

In some embodiments, the gene product is a target protein or a functional variant thereof the target transcript.

In some embodiments, the pharmaceutical composition of the present application can be administrated through any suitable routes including, for example, oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments, the pharmaceutical composition of the present application can be administered in a local rather than systemic manner, for example, via injection of the pharmaceutical composition directly into a targeted tissue. Local delivery can be affected in various ways, depending on the tissue to be targeted.

In some embodiments, the composition of the present application can be injected into the site of injury, disease manifestation, or pain, for example. In some embodiments, provided herein is a method for potent delivery to a cell of a subject comprising administrating to the subject the pharmaceutical composition as described in the present application. In some embodiments of the method, the pharmaceutical composition comprises a therapeutic agent assembled with a lipid composition as described in the present application, wherein the lipid composition comprises a lipidoid.

In some embodiments of any method described herein, the method of delivery of a therapeutic agent to a liver cell comprising administering a composition described herein, thereby providing an effective amount or activity of the therapeutic agent in the liver cell of the subject that is at least 1.1-fold greater than a corresponding amount or activity of the therapeutic agent achieved in a non-liver cell of the subject. In some embodiments, the effective amount or activity of the therapeutic agent in the liver cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of the therapeutic agent achieved in a non-liver cell of the subject.

In some embodiments, the methods of delivery comprise administering a lipid composition described herein provides an effective amount or activity of a therapeutic agent at least 1.1-fold greater than a corresponding amount or activity of the therapeutic agent achieved by administering other compositions. In some embodiments, the effective amount or activity of the therapeutic agent results from administering a lipid composition described herein is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of the therapeutic agent achieved by administering other compositions.

### Pharmaceutical Compositions

The compositions and methods of the present invention may be utilized to treat an individual in need thereof. The pharmaceutical composition described herein may comprise a therapeutic or prophylactic composition, or any combination thereof. In certain embodiments, the lipidoid compositions may be assembled with an antigen, an immune modulator, or any combination thereof. In certain embodiments, the individual is a mammal such as a human, or a non-human mammal. When administered to an animal, such as a human, the composition or the lipidoid composition is preferably administered as a pharmaceutical composition comprising, for example, a lipidoid composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition such as a lipidoid composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In certain embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896 (all of which are incorporated by reference), as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a lipidoid (e.g., nanoparticle) composition as described herein of the present invention as an active ingredient. Lipidoid compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium lipidoid compositions; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered lipidoid composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active lipidoid compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active lipidoid composition may be mixed under sterile conditions with a pharmaceutically acceptable carrier, and with any preservatives, buffers, or propellants that may be required.

The ointments, pastes, creams and gels may contain, in addition to an active lipidoid composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active lipidoid composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a lipidoid composition of the present invention to the body. Such dosage forms can be made by dissolving or dispersing the active lipidoid composition in the proper medium. Absorption enhancers can also be used to increase the flux of the lipidoid composition across the skin. The rate of such flux can be controlled by either providing a rate controlling membrane or dispersing the lipidoid composition in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the invention will be that amount of the lipidoid composition that is the lowest dose effective to produce a therapeutic effect. Such an effective dose will generally depend upon the factors described above.

If desired, the effective daily dose of the active lipidoid composition may be administered as one, two, three, four, five, six or more doses administered separately at appropriate intervals throughout the day, optionally, in unit dosage forms. In certain embodiments of the present invention, the active lipidoid composition may be administered two or three times daily. In preferred embodiments, the active lipidoid composition will be administered once daily.

The patient receiving this treatment is any animal in need, including primates, in particular humans; and other mammals such as equines, cattle, swine, sheep, cats, and dogs; poultry; and pets in general.

In certain embodiments, lipidoid compositions of the invention may be used alone or conjointly administered with another type of therapeutic agent.

The present disclosure includes the use of pharmaceutically acceptable salts of lipidoid compositions of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Polynucleotides

The pharmaceutical compositions of the present application comprise payloads (e.g., pharmaceutical agent, therapeutic agent, or prophylactic agent) assembled with the lipid composition provided herein. In some embodiments, the payloads (e.g., pharmaceutical agent, therapeutic agent, or prophylactic agent) comprise one or more polynucleotides. In some embodiments, the polynucleotides are mRNAs encoding proteins. In some embodiments, the polynucleotides are short RNAs (e.g., sgRNA, siRNA, etc.) The polynucleotides can comprise mRNA encoding a viral antigen for vaccination (e.g., a spike protein of SARS-CoV2), mRNA encoding uridine diphosphoglucuronate glucuronosyltransferase (UGT1A1), or mRNA encoding a protein for gene editing (e.g. an adenine base editor; a CRISPR-associated protein). In some embodiments, the polynucleotides comprise mRNA encoding an adenine base editor and sgRNA targeting the Angptl3 gene in a subject. The present application is not limited in scope to any particular source, sequence, or type of polynucleotide; however, as one of ordinary skill in the art could readily identify related homologs in various other sources of the polynucleotide including nucleic acids from non-human species (e.g., mouse, rat, rabbit, dog, monkey, gibbon, chimp, ape, baboon, cow, pig, horse, sheep, cat and other species). It is contemplated that the polynucleotide used in the present application can comprises a sequence based upon a naturally occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally occurring sequence. In another embodiment, the polynucleotide comprises nucleic acid sequence that is a complementary sequence to a naturally occurring sequence, or complementary to 75%, 80%, 85%, 90%, 95% and 100%. Longer polynucleotides encoding 250, 500, 1000, 1212, 1500, 2000, 2500, 3000 or longer are contemplated herein.

In some embodiments, the polynucleotide used herein may be derived from genomic DNA, i.e., cloned directly from the genome of a particular organism. In preferred embodiments, however, the polynucleotide would comprise complementary DNA (cDNA). Also contemplated is a cDNA plus a natural intron or an intron derived from another gene; such engineered molecules are sometime referred to as “mini-genes.” At a minimum, these and other nucleic acids of the present application may be used as molecular weight standards in, for example, gel electrophoresis. The term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the polynucleotide comprises one or more segments comprising a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a or double stranded ribonucleic acid (dsRNA). In some embodiments, the polynucleotide encodes at least one of the therapeutic agents (or prophylactic agent) described herein. In some embodiments, the polynucleotide encodes at least one guide polynucleotide, such as guide RNA (gRNA) or guide DNA (gDNA), for complexing with a guide RNA guided nuclease described herein. In some embodiments, the polynucleotide encodes at least one guide polynucleotide guided heterologous nuclease. The nuclease may be an endonuclease. Non-limiting example of the guide polynucleotide guided heterologous endonuclease may be selected from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof.

Some embodiments of the therapeutic agent (or prophylactic agent) provided herein comprise a heterologous polypeptide comprising an actuator moiety. The actuator moiety can be configured to complex with a target polynucleotide corresponding to a target gene. In some embodiments, administration of the therapeutic agent (or prophylactic agent) results in a modified expression or activity of the target gene. The therapeutic agent (or prophylactic agent) may comprise a heterologous polynucleotide encoding an actuator moiety. The actuator moiety may be configured to complex with a target polynucleotide corresponding to a target gene. The heterologous polynucleotide may encode a guide polynucleotide configured to direct the actuator moiety to the target polynucleotide. The actuator moiety may comprise a heterologous endonuclease or a fragment thereof (e.g., directed by a guide polynucleotide to specifically bind the target polynucleotide). The heterologous endonuclease may be (1) part of a ribonucleoprotein (RNP) and (2) complexed with the guide polynucleotide. The heterologous endonuclease may be part of a clustered regularly interspaced short palindromic repeats (CRISPR)/CRISPR-associated (Cas) protein complex. The heterologous endonuclease may be a clustered regularly interspaced short palindromic repeats (CRISPR)-associated (Cas) endonuclease. The heterologous endonuclease may comprise a deactivated endonuclease. The deactivated endonuclease may be fused to a regulatory moiety. The regulatory moiety may comprise a transcription activator, a transcription repressor, an epigenetic modifier, or a fragment thereof.

In some embodiments, the polynucleotide encodes at least one guide polynucleotide (such as guide RNA (gRNA) or guide DNA (gDNA)) guided heterologous endonuclease. In some embodiments, the polynucleotide encodes at least one guide polynucleotide and at least one heterologous endonuclease, where the guide polynucleotide can be complexed with and guides the at least one heterologous endonuclease to cleave a genetic locus of any one of the genes described herein. In some embodiments, the polynucleotide encodes at least one guide polynucleotide guided heterologous endonuclease such as Cas9, Cas12, Cas13, Cpf1 (or Cas12a), C2C1, C2C2 (or Cas13a), Cas13b, Cas13c, Cas13d, Cas14, C2C3, Casl, CaslB, Cas2, Cas3, Cas4, Cas5, Cas5e (CasD), Cas6, Cas6e, Cas6f, Cas7, Cas8a, Cas8a1, Cas8a2, Cas8b, Cas8c, Csnl, Csxl2, Cas10, Cas10d, CaslO, CaslOd, CasF, CasG, CasH, Csy1, Csy2, Csy3, Csel (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, CsxlO, Csx16, CsaX, Csx3, Csx1, Csx15, Csf1, Csf2, Csf3, Csf4, or Cul966; any derivative thereof; any variant thereof; or any fragment thereof. In some embodiments, Cas13 can include, but are not limited to, Cas13a, Cas13b, Cas13c, and Cas 13d (e.g., CasRx).

In some embodiments, the heterologous endonuclease comprises a deactivated endonuclease, optionally fused to a regulatory moiety, such as an epigenetic modifier to remodel the epigenome that mediates the expression of the selected genes of interest. In some cases, the epigenetic modifier can include methyltransferase, demethylase, dismutase, an alkylating enzyme, depurinase, oxidase, photolyase, integrase, transposase, recombinase, polymerase, ligase, helicase, glycosylase, acetyltransferase, deacetylase, kinase, phosphatase, ubiquitin-activating enzymes, ubiquitin-conjugating enzymes, ubiquitin ligase, deubiquitinating enzyme, adenylate-forming enzyme, AMPylator, de-AMPylator, SUMOylating enzyme, deSUMOylating enzyme, ribosylase, deribosylase, N-myristoyltransferase, chromotine remodeling enzyme, protease, oxidoreductase, transferase, hydrolase, lyase, isomerase, synthase, synthetase, or demyristoylation enzyme. In some instances, the epigenetic modifier can comprise one or more selected from the group consisting of p300, TET1, LSD1, HDAC1, HDAC8, HDAC4, HDAC11, HDT1, SIRT3, HST2, CobB, SIRT5, SIR2A, SIRT6, NUE, vSET, SUV39H1, DIM5, KYP, SUVR4, Set4, Set1, SETD8, and TgSET8.

In some embodiments, the polynucleotide encodes a guide polynucleotide (such as guide RNA (gRNA) or guide DNA (gDNA)) that is at least partially complementary to the genomic region of a gene, where upon binding of the guide polynucleotide to the gene the guide polynucleotide recruits the guide polynucleotide guided nuclease to cleave and genetically modified the region. Examples of the genes that may be modified by the guide polynucleotide guided nuclease include CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, or FBN1.

In some embodiments, the polynucleotide comprises or encodes at least one mRNA that, upon expression of the mRNA, restores the function of a defective gene in a subject being treated by the pharmaceutical composition described herein.

In some embodiments, the polynucleotides of the present application comprise at least one chemical modifications of the one or more nucleotides. In some embodiments, the chemical modification increases specificity of the guide polynucleotide (such as guide RNA (gRNA) or guide DNA (gDNA)) binding to a complementary genomic locus (e.g., the genomic locus of any one of the genes described herein). In some embodiments, the at least one chemical modification increases resistance to nuclease digestion, when then polynucleotide is administered to a subject in need thereof. In some embodiments, the at least one chemical modification decreases immunogenicity, when then polynucleotide is administered to a subject in need thereof. In some embodiments, the at least one chemical modification stabilizes scaffold such as a tRNA scaffold. Such chemical modification may have desirable properties, such as enhanced resistance to nuclease digestion or increased binding affinity with a target genomic locus relative to a polynucleotide without the at least one chemical modification.

In some embodiments, the at least one chemical modification comprises modification to sugar moiety. In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′—OCH₃ (“OMe” or “O-methyl”), and 2′-O(CH₂)₂OCH₃ (“MOE”). In some embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₙ), and O—CH₂—C(═O)—N(Rₘ)(Rₙ), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties.

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—,—[C(Rₐ)(R_{b})]ₙ—O—,—C(RₐR_{b})—N(R)—O— or, —C(RₐR_{b})—O—N(R)—; 4′-CH₂-2′, 4′-(CH₂)₂-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof; 4′-C(CH₃)(CH₃)—O-2′ and analogs thereof; 4′-CH₂—N(OCH₃)-2′ and analogs thereof; 4′-CH₂—O—N(CH₃)-2′; 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group; 4′-CH₂—C(H)(CH₃)-2′; and 4′-CH₂—C(═CH₂)-2′ and analogs thereof.

In some embodiments, such 4′ to 2′ bridges independently comprise from 1 to 4 linked groups independently selected from —[C(Rₐ)(R_{b})]ₙ—,—C(Rₐ)—C(R_{b})—,—C(Rₐ)═N—, —C(═NRₐ)—, —C(═O)—, —C(═S)—,—O—,—Si(Rₐ)₂—, —S(═O)ₓ—, and —N(R^{a})—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂-N(R)-2′) BNA, (I) methyl carbocyclic (4′-CH₂—CH(CH₃)-2′) BNA, (J) propylene carbocyclic (4′-(CH₂)₃₋₂′) BNA, and (K) Methoxy (ethyleneoxy) (4′-CH(CH₂OMe)-O-2′) BNA (also referred to as constrained MOE or cMOE).

In some embodiments, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH₂—O-2′) bicyclic nucleosides have been incorporated into antisense polynucleotides that showed antisense activity.

In some embodiments, substituted sugar moieties comprise one or more non-bridging sugar substituent and one or more bridging sugar substituent (e.g., 5′-substituted and 4′-2′ bridged sugars, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfur, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position and/or the 5′ position. By way of additional example, carbocyclic bicyclic nucleosides having a 4′-2′ bridge have been described.

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA), and fluoro HNA (F-HNA).

Many other bicyclo and tricyclo sugar surrogate ring systems are also known in the art that can be used to modify nucleosides for incorporation into antisense compounds.

Combinations of modifications are also provided without limitation, such as 2′-F-5′-methyl substituted nucleosides and replacement of the ribosyl ring oxygen atom with S and further substitution at the 2′-position or alternatively 5′-substitution of a bicyclic nucleic acid. In some embodiments, a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described.

In some embodiments, the present application provides polynucleotide comprising modified nucleosides. Those modified nucleotides may include modified sugars, modified nucleobases, and/or modified linkages. The specific modifications are selected such that the resulting polynucleotide possesses desirable characteristics. In some embodiments, polynucleotide comprises one or more RNA-like nucleosides. In some embodiments, polynucleotide comprises one or more DNA-like nucleotides.

In some embodiments, nucleosides of the present application comprise one or more unmodified nucleobases. In some embodiments, nucleosides of the present application comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine ([5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g., 9-(2-aminoethoxy)-H-pyrimido[5,4-13][1,4]benzoxazin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

In some embodiments, the present application provides polynucleotide comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The polynucleotides described herein contain one or more asymmetric centers and thus give rise to enantiomers, diastereomers, and other stereoisomeric configurations that may be defined, in terms of absolute stereochemistry, as (R) or(S), α or β such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated polynucleotides of the present application involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

In some embodiments, the polynucleotides described herein comprise or encode at least one tRNA described herein. In some embodiments, the tRNA expressed from the polynucleotide restores the function of at least one defective tRNA in a subject who is being treated by the pharmaceutical composition described herein. In some embodiments, the at least one tRNA expressed by the polynucleotide described herein may include tRNA that encodes alanine, arginine, asparagine, aspartic acid, cysteine, glutamic acid, glutamine, glycine, histidine, hydroxyproline, isoleucine, leucin, lysine, methionine, phenylaniline, proline, pyroglutamic acid, serine, threonine, tryptophan, tyrosine, or valine. In some embodiments, the at least one tRNA expressed by the polynucleotide described herein may include tRNA that encodes arginine, tryptophan, glutamic acid, glutamine, serine, tyrosine, lysine, leucine, glycine, or cysteine.

### Polypeptides

In some embodiments of the pharmaceutical compositions of the present application, the payload (e.g., pharmaceutical agent, therapeutic agent, or prophylactic agent) assembled with the lipid composition comprises one or more one or more polypeptides. Some polypeptides may include enzymes that can edit bases of the genome, such as an adenine base editor (e.g., ABE). In some embodiments, the polypeptide is an adenine base editor 8e (e.g., ABE8e). Some polypeptides may include enzymes such as any one of the nuclease enzymes described herein. For example, the nuclease enzyme may include from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof. In some embodiments, the nuclease enzyme may include Cas proteins such as Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein may be complexed with a guide polynucleotide described herein to be form a CRISPR ribonucleoprotein (RNP).

The nuclease in the compositions described herein may be Cas9 (e.g., from S. pyogenes or S. pneumonia). The CRISPR enzyme can direct cleavage of one or both strands at the location of a target sequence, such as within the target sequence and/or within the complement of the target sequence of any one of the genes described herein.

The CRISPR enzyme may be mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, the present application provides polypeptide containing one or more therapeutic proteins. The therapeutic proteins that may be included in the composition include a wide range of molecules such as cytokines, chemokines, interleukins, interferons, growth factors, coagulation factors, anti-coagulants, blood factors, bone morphogenic proteins, immunoglobulins, and enzymes. Some non-limiting examples of particular therapeutic proteins include Erythropoietin (EPO), Granulocyte colony-stimulating factor (G-CSF), Alpha-galactosidase A, Alpha-L-iduronidase, Thyrotropin α, N-acetylgalactosamine-4-sulfatase (rhASB), Dornase alfa, Tissue plasminogen activator (TPA) Activase, Glucocerebrosidase, Interferon (IF) β-1a, Interferon β-1b, Interferon γ, Interferon α, TNF-α, IL-1 through IL-36, Human growth hormone (rHGH), Human insulin (BHI), Human chorionic gonadotropin α, Darbepoetin α, Follicle-stimulating hormone (FSH), and Factor VIII.

In some embodiments, the polypeptide comprises a peptide sequence that is at least partially identical to any of the therapeutic agent (or prophylactic agent) comprising a peptide sequence. For example, the polypeptide may comprise a peptide sequence that is at least partially identical to an antibody (e.g., a monoclonal antibody) for treating a disease such as cancer.

In some embodiments, the polypeptide comprises a peptide or protein that restores the function of a defective protein in a subject being treated by the pharmaceutical composition described herein.

In some embodiments, the pharmaceutical composition of the present application comprises a plurality of payloads assembled with (e.g., encapsulated within) a lipid composition. The plurality of payloads assembled with the lipid composition may be configured for gene-editing or gene-expression modification. The plurality of payloads assembled with the lipid composition may comprise a polynucleotide encoding an actuator moiety (e.g., comprising a heterologous endonuclease such as Cas) or a polynucleotide encoding the actuator moiety. The plurality of payloads assembled with the lipid composition may further comprise one or more (e.g., one or two) guide polynucleotides. The plurality of payloads assembled with the lipid composition may further comprise one or more donor or template polynucleotides. The plurality of payloads assembled with the lipid composition may comprise a ribonucleoprotein (RNP).

In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no more than about 20:1, no more than about 15:1, no more than about 10:1, or no more than about 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no less than about 20:1, no less than about 15:1, no less than about 10:1, or no less than about 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 10:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 10:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 15:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1.

In some embodiments of the pharmaceutical composition of the present application, a molar ratio of the therapeutic agent to total lipids of the lipid composition is from about 1:1 to about 1:100. In some embodiments of the pharmaceutical composition of the present application, a molar ratio of the therapeutic agent to total lipids of the lipid composition is from about 1:1 to about 1:50. In some embodiments of the pharmaceutical composition of the present application, a molar ratio of the therapeutic agent to total lipids of the lipid composition is from about 50:1 to about 1:100. In some embodiments of the pharmaceutical composition of the present application, a molar ratio of the therapeutic agent to total lipids of the lipid composition is from about 1:1 to about 1:20. In some embodiments of the pharmaceutical composition of the present application, a molar ratio of the therapeutic agent to total lipids of the lipid composition is from about 20:1 to about 1:50. In some embodiments of the pharmaceutical composition of the present application, a molar ratio of the therapeutic agent to total lipids of the lipid composition is from about 50:1 to about 1:70. In some embodiments of the pharmaceutical composition of the present application, a molar ratio of the therapeutic agent to total lipids of the lipid composition is from about 70:1 to about 1:100. In some embodiments of the pharmaceutical composition of the present application, a molar ratio of the therapeutic agent to total lipids of the lipid composition is no more than about 1:1, no more than about 1:5, no more than about 1:10, no more than about 1:15, no more than about 1:20, no more than about 1:25, no more than about 1:30, no more than about 1:35, no more than about 1:40, no more than about 1:45, no more than about 1:50, no more than about 1:60, no more than about 1:70, no more than about 1:80, no more than about 1:90, or more than about 1:100. In some embodiments of the pharmaceutical composition of the present application, a molar ratio of the therapeutic agent to total lipids of the lipid composition is no less than about 1:1, no less than about 1:5, no less than about 1:10, no less than about 1:15, no less than about 1:20, no less than about 1:25, no less than about 1:30, no less than about 1:35, no less than about 1:40, no less than about 1:45, no less than about 1:50, no less than about 1:60, no less than about 1:70, no less than about 1:80, no less than about 1:90, or less than about 1:100.

In some embodiments of the pharmaceutical composition of the present application, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of the therapeutic agent is encapsulated in particles of the lipid compositions.

In some embodiments of the pharmaceutical composition of the present application, the lipid composition comprises a plurality of particles characterized by one or more characteristics of the following: (1) a (e.g., average) size of 100 nanometers (nm) or less; (2) a polydispersity index (PDI) of no more than about 0.2; and (3) a zeta potential of −10 millivolts (mV) to 10 mV.

In some embodiments of the pharmaceutical composition of the present application, the lipid composition comprises a plurality of particles with a (e.g., average) size from about 50 nanometers (nm) to about 100 nanometers (nm). In some embodiments of the pharmaceutical composition of the present application, the lipid composition comprises a plurality of particles with a (e.g., average) size from about 70 nanometers (nm) to about 100 nanometers (nm). In some embodiments of the pharmaceutical composition of the present application, the lipid composition comprises a plurality of particles with a (e.g., average) size from about 50 nanometers (nm) to about 80 nanometers (nm). In some embodiments of the pharmaceutical composition of the present application, the lipid composition comprises a plurality of particles with a (e.g., average) size from about 60 nanometers (nm) to about 80 nanometers (nm). In some embodiments of the pharmaceutical composition of the present application, the lipid composition comprises a plurality of particles with a (e.g., average) size of at most about 100 nanometers (nm), at most about 90 nanometers (nm), at most about 85 nanometers (nm), at most about 80 nanometers (nm), at most about 75 nanometers (nm), at most about 70 nanometers (nm), at most about 65 nanometers (nm), at most about 60 nanometers (nm), at most about 55 nanometers (nm), or at most about 50 nanometers (nm). In some embodiments of the pharmaceutical composition of the present application, the lipid composition comprises a plurality of particles with a (e.g., average) size of at least about 100 nanometers (nm), at least about 90 nanometers (nm), at least about 85 nanometers (nm), at least about 80 nanometers (nm), at least about 75 nanometers (nm), at least about 70 nanometers (nm), at least about 65 nanometers (nm), at least about 60 nanometers (nm), at least about 55 nanometers (nm), or at least about 50 nanometers (nm). The (e.g., average) size may be determined by size exclusion chromatography (SEC). The (e.g., average) size may be determined by spectroscopic method(s) or image-based method(s), for example, dynamic light scattering, static light scattering, multi-angle light scattering, laser light scattering, or dynamic image analysis, or a combination thereof.

In some embodiments of the pharmaceutical composition of the present application, the lipid composition comprises a plurality of particles with a polydispersity index (PDI) from about 0.05 to about 0.5. In some embodiments of the pharmaceutical composition of the present application, the lipid composition comprises a plurality of particles with a polydispersity index (PDI) from about 0.1 to about 0.5. In some embodiments of the pharmaceutical composition of the present application, the lipid composition comprises a plurality of particles with a polydispersity index (PDI) from about 0.1 to about 0.3. In some embodiments of the pharmaceutical composition of the present application, the lipid composition comprises a plurality of particles with a polydispersity index (PDI) from about 0.2 to about 0.5. In some embodiments of the pharmaceutical composition of the present application, the lipid composition comprises a plurality of particles with a polydispersity index (PDI) of no more than about 0.5, no more than about 0.4, no more than about 0.3, no more than about 0.2, no more than about 0.1, or no more than about 0.05.

In some embodiments of the pharmaceutical composition of the present application, the lipid composition comprises a plurality of particles with a negative zeta potential of −5 millivolts (mV) or less. In some embodiments of the pharmaceutical composition of the present application, the lipid composition comprises a plurality of particles with a negative zeta potential of −10 millivolts (mV) or less. In some embodiments of the pharmaceutical composition of the present application, the lipid composition comprises a plurality of particles with a negative zeta potential of −15 millivolts (mV) or less. In some embodiments of the pharmaceutical composition of the present application, the lipid composition comprises a plurality of particles with a negative zeta potential of −20 millivolts (mV) or less. In some embodiments of the pharmaceutical composition of the present application, the lipid composition comprises a plurality of particles with a negative zeta potential of −30 millivolts (mV) or less. In some embodiments, the lipid composition comprises a plurality of particles with a zeta potential of 0 millivolts (mV) or less. In some embodiments, the lipid composition comprises a plurality of particles with a zeta potential of 5 millivolts (mV) or less. In some embodiments, the lipid composition comprises a plurality of particles with a zeta potential of 10 millivolts (mV) or less. In some embodiments of the pharmaceutical composition of the present application, the lipid composition comprises a plurality of particles with a negative zeta potential of 15 millivolts (mV) or less. In some embodiments of the pharmaceutical composition of the present application, the lipid composition comprises a plurality of particles with a negative zeta potential of 20 millivolts (mV) or less.

In some embodiments of the pharmaceutical composition of the present application, the lipid composition comprises a plurality of particles with a negative zeta potential of −5 millivolts (mV) or more. In some embodiments of the pharmaceutical composition of the present application, the lipid composition comprises a plurality of particles with a negative zeta potential of −10 millivolts (mV) or more. In some embodiments of the pharmaceutical composition of the present application, the lipid composition comprises a plurality of particles with a negative zeta potential of −15 millivolts (mV) or more. In some embodiments of the pharmaceutical composition of the present application, the lipid composition comprises a plurality of particles with a negative zeta potential of −20 millivolts (mV) or more. In some embodiments of the pharmaceutical composition of the present application, the lipid composition comprises a plurality of particles with a negative zeta potential of −30 millivolts (mV) or more. In some embodiments, the lipid composition comprises a plurality of particles with a zeta potential of 0 millivolts (mV) or more. In some embodiments, the lipid composition comprises a plurality of particles with a zeta potential of 5 millivolts (mV) or more. In some embodiments, the lipid composition comprises a plurality of particles with a zeta potential of 10 millivolts (mV) or more. In some embodiments of the pharmaceutical composition of the present application, the lipid composition comprises a plurality of particles with a zeta potential of 15 millivolts (mV) or more. In some embodiments of the pharmaceutical composition of the present application, the lipid composition comprises a plurality of particles with a zeta potential of 20 millivolts (mV) or more.

In some embodiments of the pharmaceutical composition of the present application, the lipid composition has an apparent ionization constant (pKa) outside a range of 6 to 7. In some embodiments of the pharmaceutical composition of the present application, the lipid composition has an apparent pKa of about 8 or higher, about 9 or higher, about 10 or higher, about 11 or higher, about 12 or higher, or about 13 or higher. In some embodiments of the pharmaceutical composition of the present application, the lipid composition has an apparent pKa of about 8 to about 13. In some embodiments of the pharmaceutical composition of the present application, the lipid composition has an apparent pKa of about 8 to about 10. In some embodiments of the pharmaceutical composition of the present application, the lipid composition has an apparent pKa of about 9 to about 11. In some embodiments of the pharmaceutical composition of the present application, the lipid composition has an apparent pKa of about 10 to about 13. In some embodiments of the pharmaceutical composition of the present application, the lipid composition has an apparent pKa of about 8 to about 12. In some embodiments of the pharmaceutical composition of the present application, the lipid composition has an apparent pKa of about 10 to about 12.

### Dosing Level

In another aspect, provided is high-potency dosage form of a pharmaceutical agent (or a therapeutic agent) formulated with a lipidoid, the dosage form comprising a therapeutic agent assembled with a lipid composition as described herein.

In some embodiments, the therapeutic agent is present in the dosage form at a dose of about 10, 9, 8, 7, 6, 5, 4, 3, 2, 1.5, 1.0, 0.5, 0.2, 0.1, 0.05, 0.02, 0.01, 0.005, 0.002, or 0.001 milligram per kilogram (mg/kg, or mpk) body weight, or of a range between (inclusive) any two of the foregoing values.

In some embodiments, the therapeutic agent is present in the dosage form at a dose of no more than about 10 milligram per kilogram (mg/kg, or mpk) body weight. In some embodiments, the therapeutic agent is present in the dosage form at a dose of no more than about 9 mg/kg, no more than about 8 mg/kg, no more than about 7 mg/kg, no more than about 6 mg/kg, no more than about 5 mg/kg, no more than about 4 mg/kg, no more than about 3 mg/kg, no more than about 2 mg/kg, no more than about 1 mg/kg, no more than about 0.5 mg/kg, no more than about 0.2 mg/kg, no more than about 0.1 mg/kg, no more than about 0.05 mg/kg, or no more than about 0.01 mg/kg. In some embodiments, the therapeutic agent is present in the dosage form at a concentration of no more than about 5 milligram per milliliter (mg/mL).

In some embodiments, the therapeutic agent is present in the dosage form at a concentration of about 5, 4, 3, 2, 1, 0.5, 0.2, or 0.1 milligram per milliliter (mg/mL), or of a range between (inclusive) any two of the foregoing values.

In some embodiments, the therapeutic agent is present in the dosage form at a concentration of no more than about 5 milligram per milliliter (mg/mL). In some embodiments, the therapeutic agent is present in the dosage form at a concentration of no more than about 2 milligram per milliliter (mg/mL). In some embodiments, the therapeutic agent is present in the dosage form at a concentration of no more than about 1 milligram per milliliter (mg/mL). In some embodiments, the therapeutic agent is present in the dosage form at a concentration of no more than about 0.5 milligram per milliliter (mg/mL). In some embodiments, the therapeutic agent is present in the dosage form at a concentration of no more than about 0.1 milligram per milliliter (mg/mL).

In some embodiments, the therapeutic agent (e.g., proteins, nucleic acids) is present in the dosage form at a concentration of about 10, 9, 8, 7, 6, 5, 4, 3, 2, 1, 0.5, 0.2, or 0.1 microgram per milliliter (μg/mL), or of a range between (inclusive) any two of the foregoing values. In some embodiments, the therapeutic agent is present in the dosage form at a concentration of no more than about 10, no more than about 9, no more than about 8, no more than about 7, no more than about 6, no more than about 5, no more than about 4, no more than about 3, no more than about 2, no more than about 1, no more than about 0.5, no more than about 0.2, no more than about 0.1 microgram per milliliter (μg/mL).

Any suitable dosage form can be prepared for delivery, for example, via oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments, the pharmaceutical agent is administered at a dosage of no more than about 10 milligram per kilogram (mg/kg, or mpk) body weight, no more than about 9 mg/kg, no more than about 8 mg/kg, no more than about 7 mg/kg, no more than about 6 mg/kg, no more than about 5 mg/kg, no more than about 4 mg/kg, no more than about 3 mg/kg, no more than about 2 mg/kg, no more than about 1 mg/kg, no more than about 0.5 mg/kg, no more than about 0.2 mg/kg, no more than about 0.1 mg/kg, no more than about 0.05 mg/kg, or no more than about 0.01 mg/kg body weight. In some embodiments, the pharmaceutical agent is administered at a dosage from about 1 μg/kg body weight to about 3 mg/kg body weight.

In some embodiments, the administration of a dose of the lipid composition provided here can be repeated. If desired, the effective dose of the active lipid composition can be administered as one, two, three, four, five, six or more doses administered separately at appropriate intervals throughout the course of treatment. In some embodiments, the lipid composition can be administered two or three times daily. In some embodiments, the lipid composition will be administered once daily. In some embodiments, the lipid composition is administered about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, or about every 18 weeks. In some embodiments, the lipid composition is administered about every 1 month, about every 2 months, about every 3 months, about every 4 months, about every 5 months, about every 6 months, about every 7 months, about every 8 months, about every 9 months, about every 10 months, about every 11 months, about every 12 months, about every 13 months, about every 14 months, about every 15 months, about every 16 months, about every 17 months, about every 18 months, about every 2 years, about every 2.5 years, about every 3 years, about every 3.5 years, about every 4 years, about every 4.5 years, or about every 5 years. Any subject in need thereof can be treated with the method of the present application. In some embodiments, the subject has been determined to likely respond to the therapeutic agent. For example, the subject may have, is suffering from, or suspected of having a disease or condition.

In some embodiments, the subject has been determined to have a (e.g., missense or nonsense) mutation in a target gene. In some embodiments, the mutation in the target gene is associated with a genetic disease or disorder.

In some embodiments, the subject has been determined to exhibit an aberrant expression or activity of a protein or polynucleotide that corresponds to a target gene. In some embodiments, the aberrant expression or activity of the protein or polynucleotide is associated with a genetic disease or disorder.

In some embodiments, the subject is selected from the group consisting of mouse, rat, monkey, and human. In some embodiments, the subject is a human.

In some embodiments, provided herein is a method for targeted delivery of a therapeutic agent to a cell type comprising contacting the cell with the pharmaceutical composition of the present application. In some embodiments of the method, the pharmaceutical composition comprises a therapeutic agent assembled with a lipid composition as described in the present application, e.g., wherein the lipid composition comprises any of the head or tail groups disclosed herein.

In some embodiments, the contacting is ex vivo. In some embodiments, the contacting is in vitro. In some embodiments, the contacting is in vivo. In some embodiments, the contacting comprises administering to a subject the composition comprising the therapeutic agent assembled with the lipid composition.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

“Alkenyl” is a branched or straight chain aliphatic hydrocarbon group having one or more carbon-carbon double bonds that may occur at any stable point along the chain, having the specified number of carbon atoms. Examples of alkenyl include, but are not limited to, ethenyl and propenyl.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “hydroxyalkyl” refers to an alkyl group having a hydroxyl attached thereto. Representative hydroxyalkyl groups include hydroxyethyl (—CH₂CH₂OH), hydroxypropyl, hydroxybutyl, hydroxypentyl, and the like.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

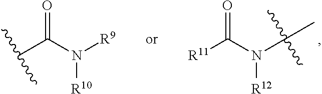

wherein R⁹, R¹⁰, R¹¹, and R¹² each independently represent a hydrogen or hydrocarbyl group, or R⁹ and R¹⁰ taken together with the N atom to which they are attached complete a heterocycle having from 4 to 8 atoms in the ring structure. If an amide is drawn without depicting a group required by valence on the constituent nitrogen atom (e.g., as in —C(O)—NR⁹), then a hydrogen atom is implied and understood to be present on the nitrogen atom to satisfy the aforementioned valence requirement (e.g., as in —C(O)—NHR⁹).

The terms “amine” and “amino” are art-recognized and refer to both unsubstituted and substituted amines and salts thereof, e.g., a moiety that can be represented by

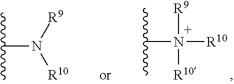

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

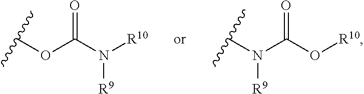

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent (C₁-C₁₀), preferably six or fewer (C₁-C₆). A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

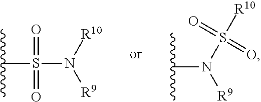

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR₉ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

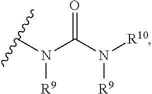

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Salt” is used herein to refer to an acid addition salt or a basic addition salt.

Many of the lipidoid compositions (e.g., nanoparticles) useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers. Some of the lipidoid compositions (e.g., nanoparticles) may also comprise chemical compound which exist in tautomeric forms. Such forms, although not explicitly indicated in the formulae described herein, are intended to be included within the scope of the present disclosure.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e. g., Berge, et al., J. Pharm. Sci. 66 (1): 1-79 (January 1977).

“Pharmaceutically acceptable vehicle” refers to a diluent, adjuvant, excipient or carrier with which a compound of the invention is administered.

“Pharmaceutically acceptable metabolically cleavable group” refers to a group that is cleaved in vivo to yield the parent molecule of the structural formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkylesters or (alkoxycarbonyl)oxy)alkylesters. Particularly the C₁-C₈ alkyl, C₂-C₈ alkenyl, C₂-C₈ alkynyl, aryl, C₇-C₁₂ substituted aryl, and C₇-C₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

The term “helper lipid” as used in this disclosure refers to a lipid that contributes to the stability or delivery efficacy of a lipid composition. A helper lipid can be a zwitterionic lipid, such as a phospholipid. A helper lipid can be phosphatidylcholine, distearoylphosphatidylcholine, dioleoylphosphatidylethanolamine, 1,2-dioleoyl-sn-glycero-3-phosphoethanolamine (DOPE) or 1,2-dioleoyl-sn-glycero-3-phosphocholine (DOPC). In some cases, the term “helper lipid” refers to phospholipids or other zwitterionic lipids in the LNP composition. In some cases, when describing the formulation of an LNP using weight ratios of the lipid components (e.g., lipidoid, steroid, helper lipid, and polymer conjugated lipid), a helper lipid refers to a phospholipid or another zwitterionic lipid. For example, the weight ratio of the lipidoid/steroid/helper lipid/polymer conjugated lipid is about 14/4/1/1. “Helper lipid” can refer to any class of lipid molecules that improves the particle stability and fluidity of lipid nanoparticles (LNP). Several classes of molecules can be used as helper lipids such as phospholipids (e.g., phosphoethanolamine, phosphocholine), zwitterionic lipids, steroid derivatives, and polymer conjugated lipids (e.g., PEGylated lipid). Representative helper lipids include cholesterol, 1,2-dioleoyl-sn-glycero-3-phosphoethanolamine (DOPE), 1,2-dioleoyl-sn-glycero-3-phosphocholine (DOPC), Phosphatidylcholine (PC), Methoxy-Polyethyleneglycol (MW 2k)-distearoylphosphatidylethanolamine (mPEG2k-DSPE), and 1,2-dimyristoyl-rac-glycero-3-methoxypolyethylene glycol-2000 (DMG-PEG2k).

A “subject” to which administration is contemplated includes, but is not limited to, humans (i.e., a male or female of any age group, e.g., a pediatric subject (e.g, infant, child, adolescent) or adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing” or “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be “²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 95% by weight R-compound and at most about 5% by weight S-compound, at least about 99% by weight R-compound and at most about 1% by weight S-compound, or at least about 99.9% by weight R-compound and at most about 0.1% by weight S-compound. In certain embodiments, the weights are based upon total weight of compound.

As used herein and unless otherwise indicated, the term “enantiomerically pure S-compound” or “S-compound” refers to at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

### Example 1: In Vivo Screening of Lipid Nanoparticles for mRNA Delivery

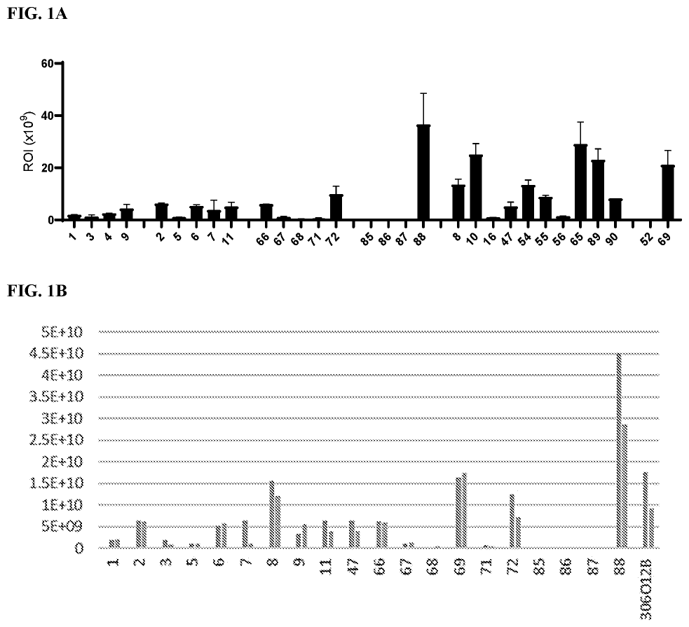

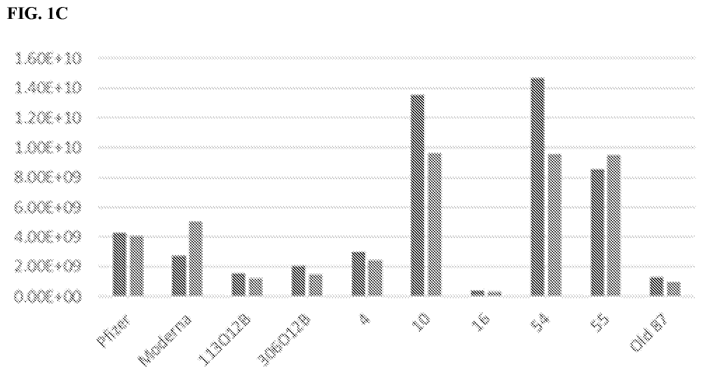

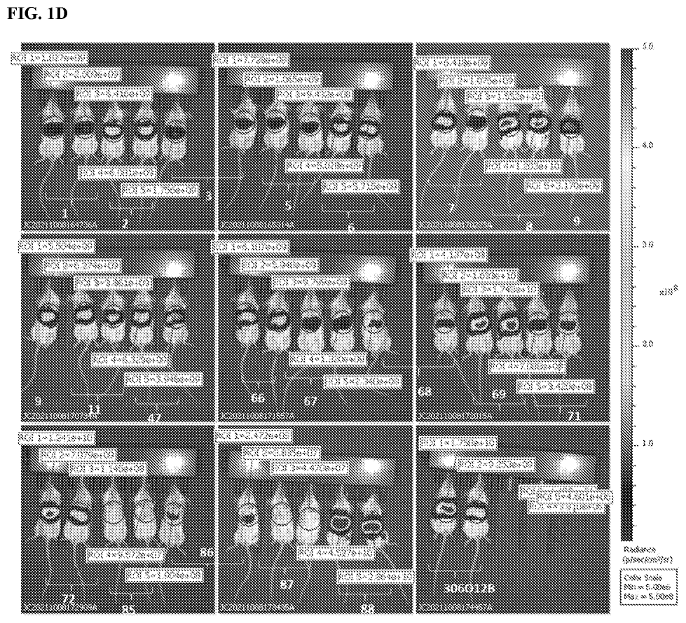

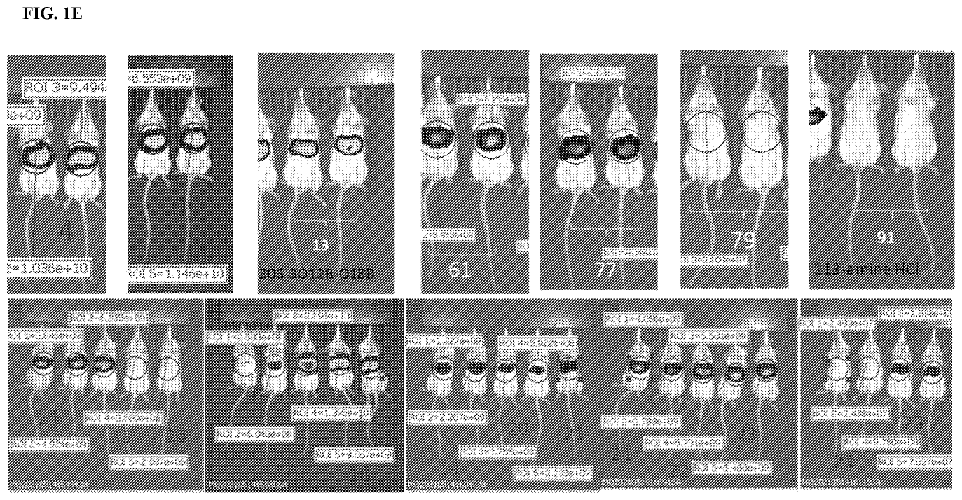

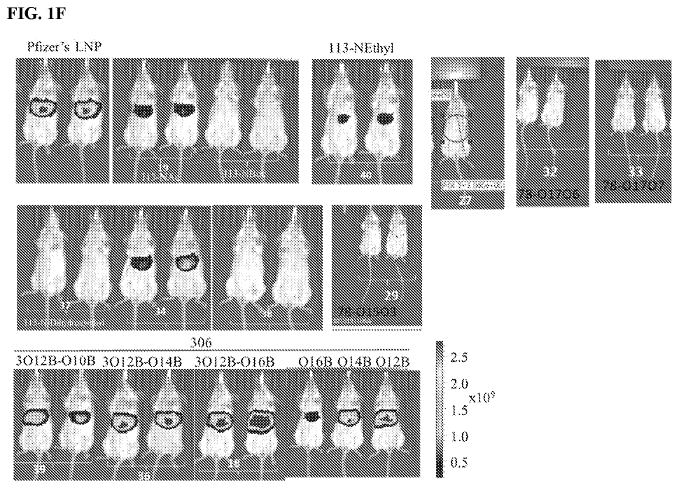

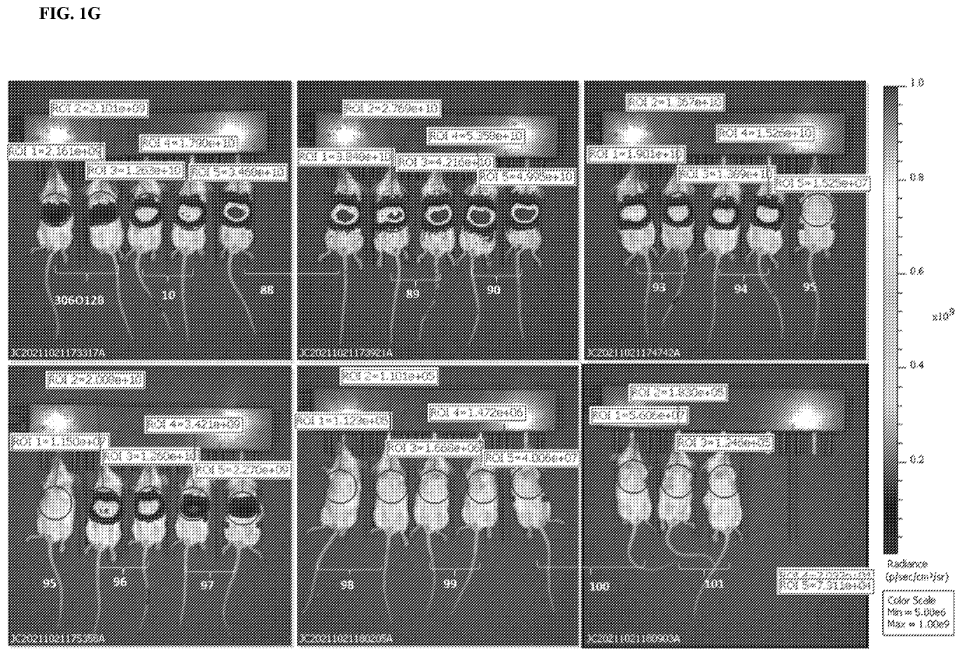

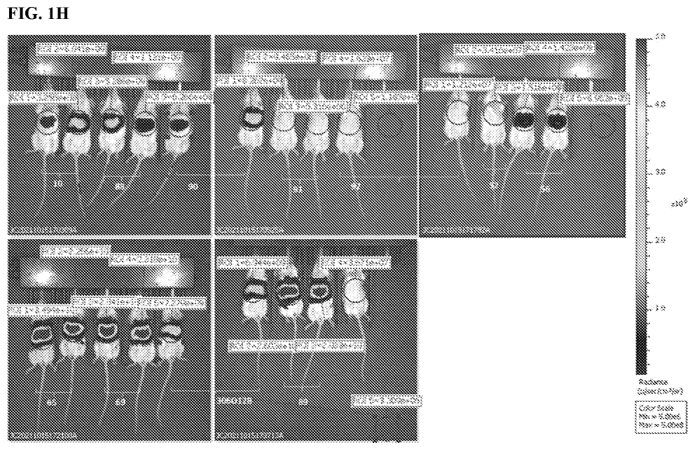

Bioreducible lipids of the present disclosure were prepared via a combinatory solvent free Michael-Addition as depicted in FIG. 1J scheme 1. Briefly, lipid tails comprising an acrylate (e.g., branched, non-branched; saturated or unsaturated) and head groups comprising an amine (e.g., ionizable) were mixed and reacted at 70° C. for 48 hours. Crude products were purified using a Teledyne Isco Chromatography system using methanol/DCM as mobile phase. The purified lipidoids were characterized by electrospray ionization mass spectrometry (ESI-MS).

The in vivo mRNA delivery efficacy of the lipids was evaluated in female wild-type Balb/c mice. Firefly luciferase mRNA (fLuc mRNA)/LNPs were formulated with the active lipids (e.g., ionizable lipids of the present disclosure), helper lipids (e.g., DSPC, DMG-PEG) and excipient compounds (e.g., cholesterol). FLuc mRNA/LNPs were administered intravenously to Balb/c mice. Six hours after mRNA/LNPs administration, luciferin substrate was given to mice via intraperitoneal injections. Whole-body fLuc activity was measured using an in vivo imaging system (e.g., IVIS by PerkinElmer).

As summarized in FIGS. 1A-1C, LNPs of the present disclosure resulted in improved f.Luc mRNA delivery to mice and higher luciferase expression in vivo in the liver.

### Example 2: Characterization of Exemplary Lipids

After formulation, mRNA/LNPs were characterized. Size and polydispersity index (PDI) were determined by dynamic light scattering. Encapsulation efficiency (EE %) was measured using a fluorescence plate-based assay. Molecular weight of exemplary lipids was also measured, as depicted in TABLE 3.

TABLE 3
Molecular weight of exemplary lipids

| Lipid No. | Observed Molecular Ions |
|---|---|
| 4 | 536.73, 592.82, 960.55, 972.55, 975.55, 1308.73 |
| 9 | 641.09, 1266.73, 1280.91 |
| 12 | 1032.73, 1033.75, 1266.62, 1267.82, 1269.78 |
| 58 | 1251.74, 1252.71, 1254.67 |
| 93 | 874.89 |
| 104 | 584.38, 1166.90 |
| 105 | 584.38 |

### Example 3: In Vivo Optimization of Lipid 88 LNP Formulations

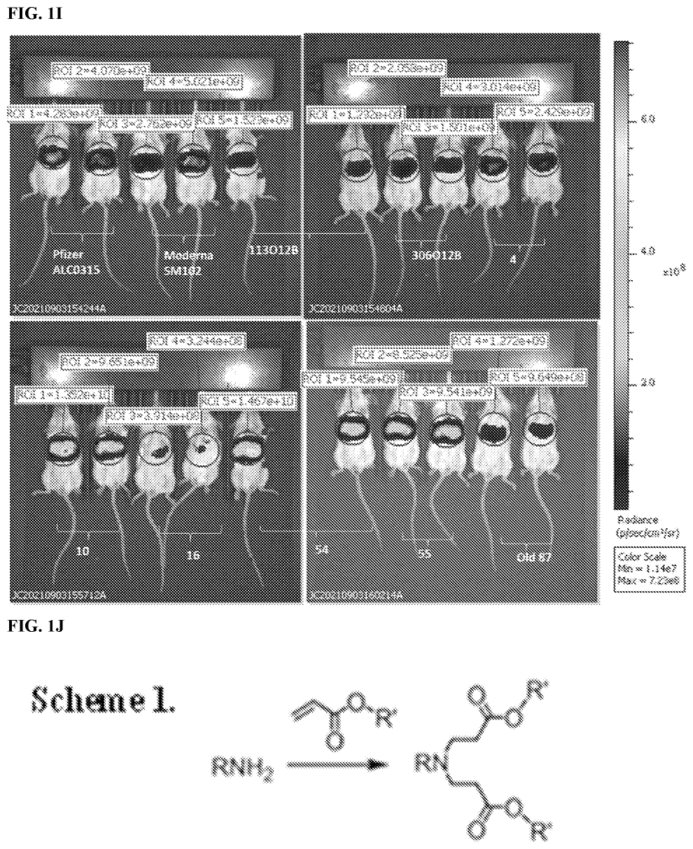

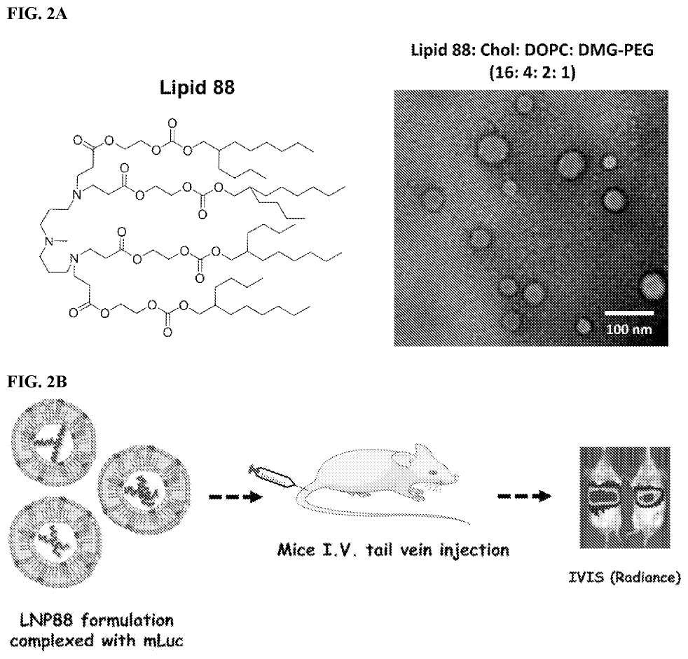

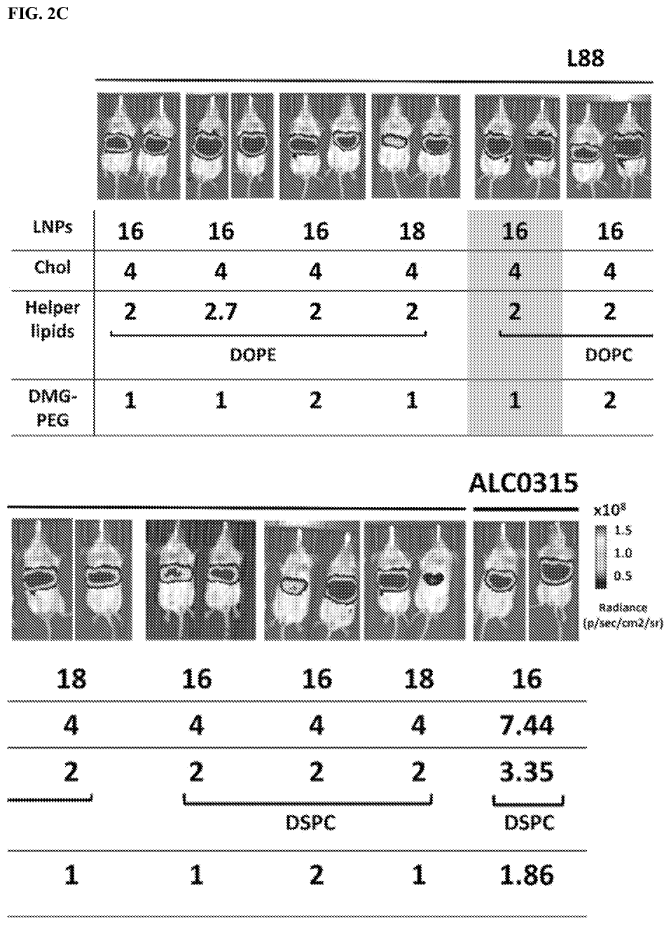

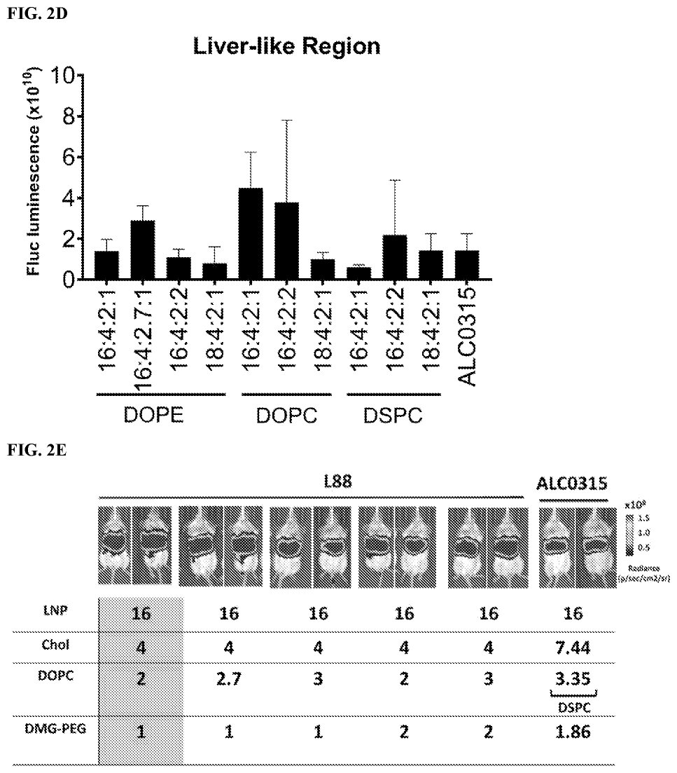

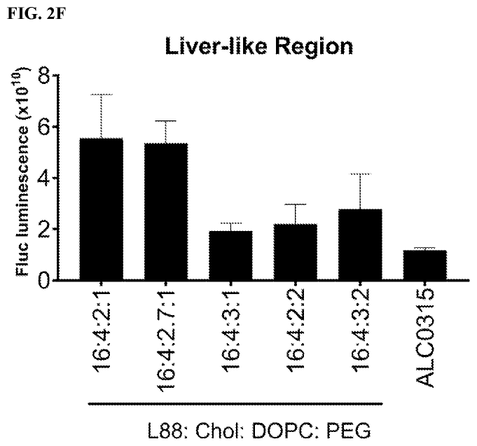

Various formulations of mRNA/LNPs comprising lipid 88 were tested in vivo, including a plurality of ratios of ionizable lipids to helper lipids, and ratios of RNA to lipids. Briefly, as showed in the schematic in FIG. 2B, SomU-Fluc mRNA, lipid 88, cholesterol, and helper lipids (e.g., DOPC, DOPE, DSPC and/or DMG-PEG) were formulated at various ratios. The luc mRNA/LNPs were then given to both adult and neonatal mice via various routes of administration (e.g., intravenously, intramuscularly, or through facial vein). After a period of time post injection (e.g., 6-80 hours), substrates of luciferase were administered to the animals and whole-body imaging was performed using IVIS imaging system.

First i.v. (intravenous) injections of various lipid formulations of luc mRNA/LNPs comprising lipid 88 were administered to adult mice, and luciferase substrates were administered 6 hours post LNPs injection. FIG. 2C and FIG. 2E show in vivo images of living adult mice after treated with i.v. mLuc/LNPs formulated with lipid 88, with a commercial lipid ALC0315 as control. As depicted in FIG. 2C and FIG. 2E, and quantified in FIG. 2D and FIG. 2F, all formulations tested showed expression of luciferase in adult animals at the liver-like region. The formulation 16:4:2:1 (L88:Chol:DOPC:DMG-PEG, w w), 16:4:2:2 (L88:Chol:DOPC:DMG-PEG, w w), and 16:4:2.7:1 (L88:Chol:DOPC:DMG-PEG, w w), showed strong expression of luciferase at the liver-like region of mice.

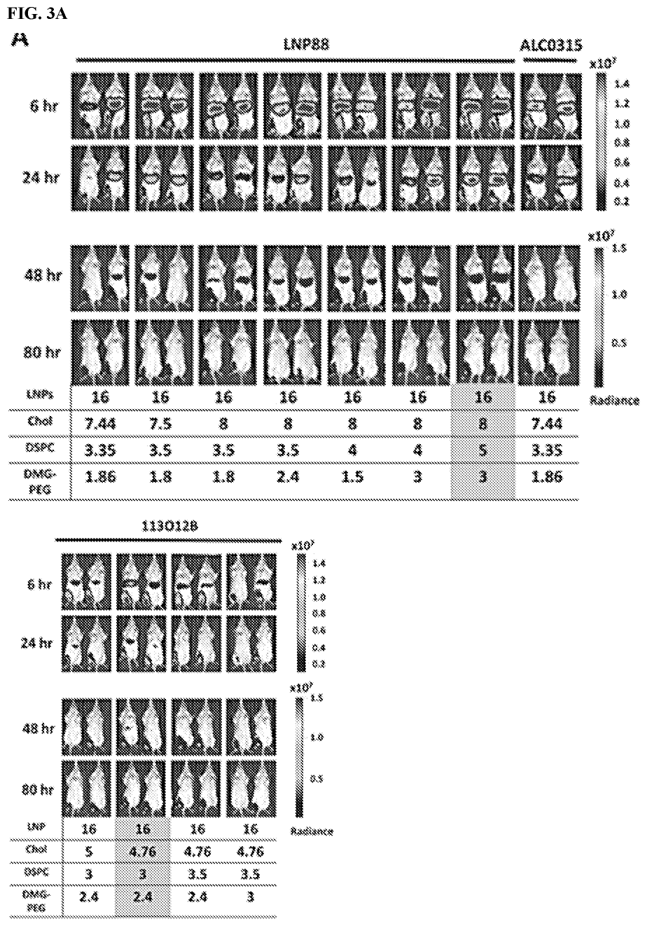

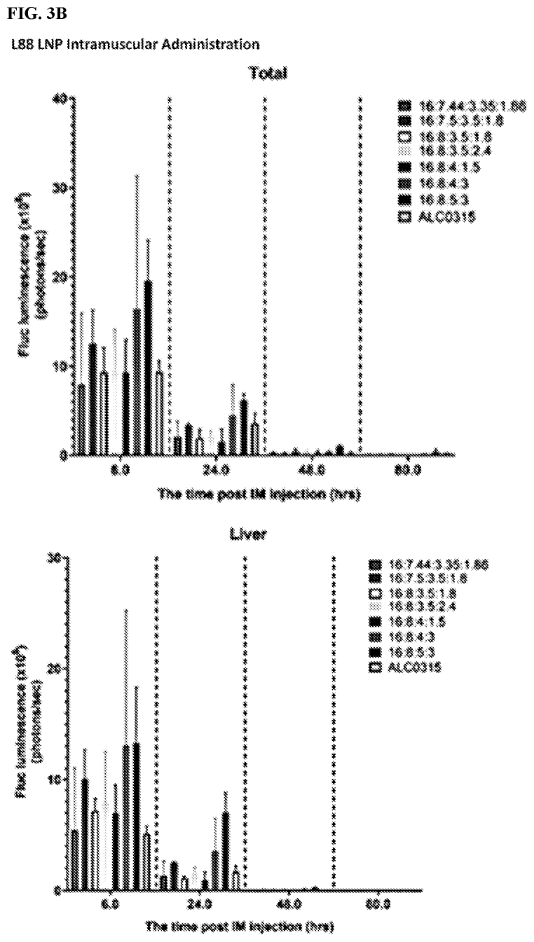

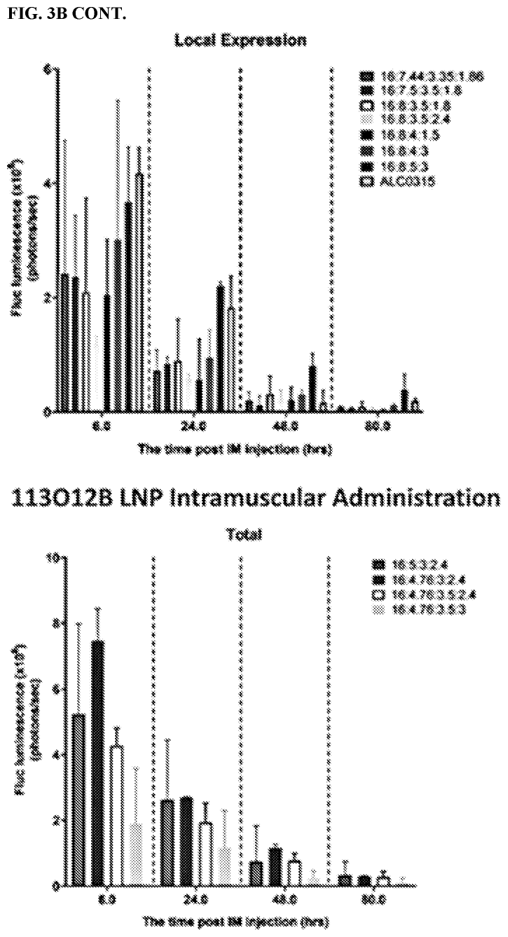

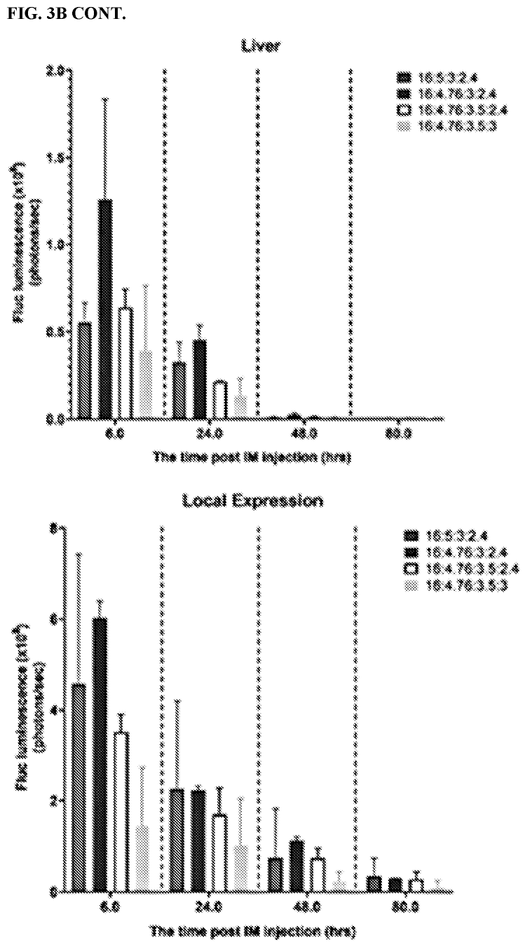

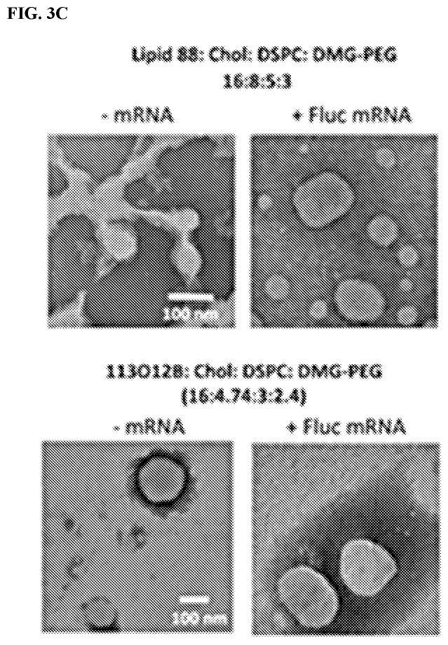

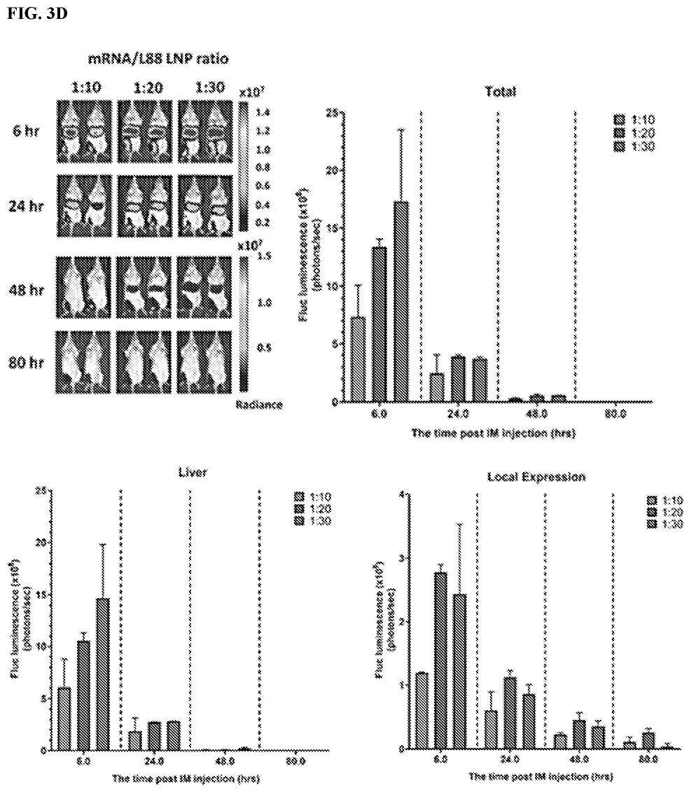

I.M (intramuscular) injections of various lipid formulations of luc mRNA/LNPs comprising lipid 88 were then tested in adult mice, and luciferase substrates were administered 6 hours to 80 hours post LNPs injection FIG. 3A shows images of in vivo bioluminescence measurements in mice from 6 hrs, 24 hrs, 48 hrs, and 80 hrs following intramuscular (n=2 mice) injection of mLuc-LNP with varying ratios of helper lipids. FIG. 3B and FIG. 3C show the quantification of bioluminescence over time in mice treated with Lipid 88 and 113O12B mRNA/LNP delivery respectively. Scale represents radiance (photo/sec/cm²/sr). IM injections of LNPs formulated with lipid 88 (e.g. comprising branched lipid tails and carbonate) resulted in enhanced delivery of luc mRNA to the mice compared with LNPs formulated with 113O12B (e.g., unbranched lipid tails). The formulation 16:8:(3.35-5):(1.4-3) (L88:Chol:DOPC:DMG-PEG, w/w) resulted in strong expression of luciferase in the mice liver. FIG. 3D shows TEM imaging of the optimized Lipid 88 and 113O12B formulations with and without mRNA loading. Scale represents 100 nm.

Next mRNA to lipid 88 LNP ratios were tested and the results are shown in FIG. 3E. Varying ratios of mLuc to LNP88 (1:10-1:30) LNPs were injected intramuscularly to mice as described, and the animals were imaged from 6 hrs to 80 hrs post injections. Formulation mLuc:LNP88 (1:20) and mLuc:LNP88 (1:30) achieved better mRNA delivery and higher expression of luciferase, as depicted in FIG. 3E.

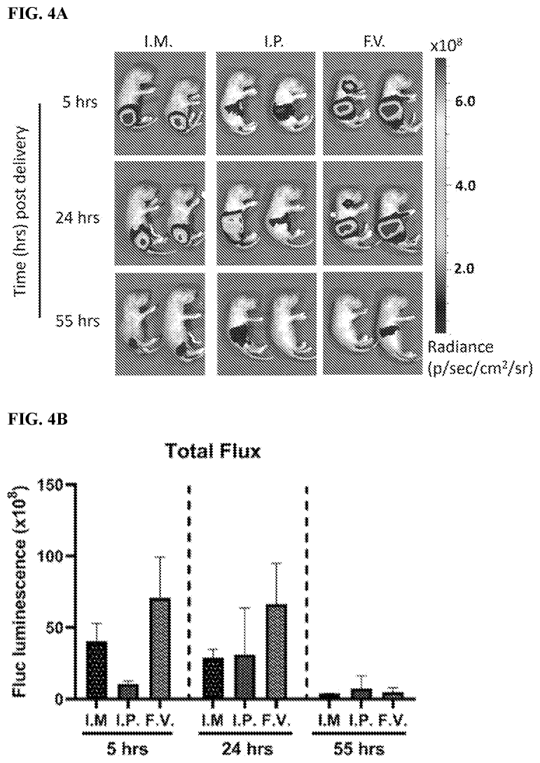

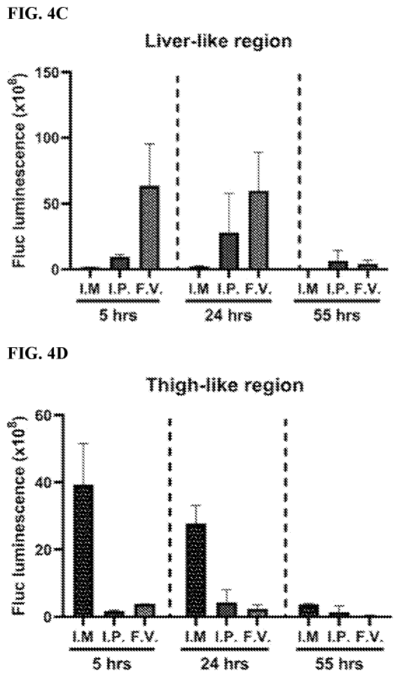

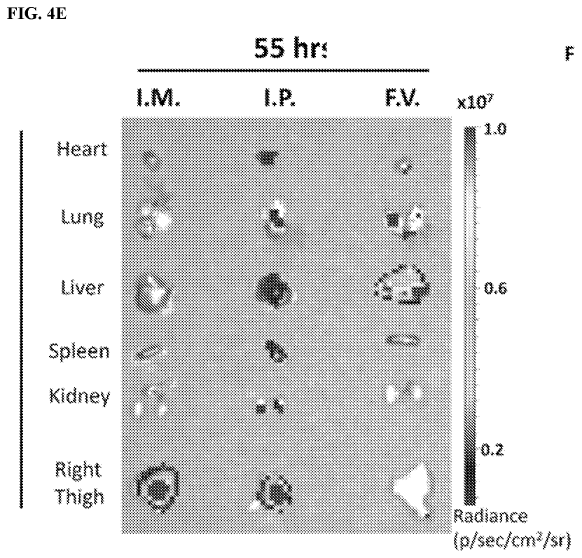

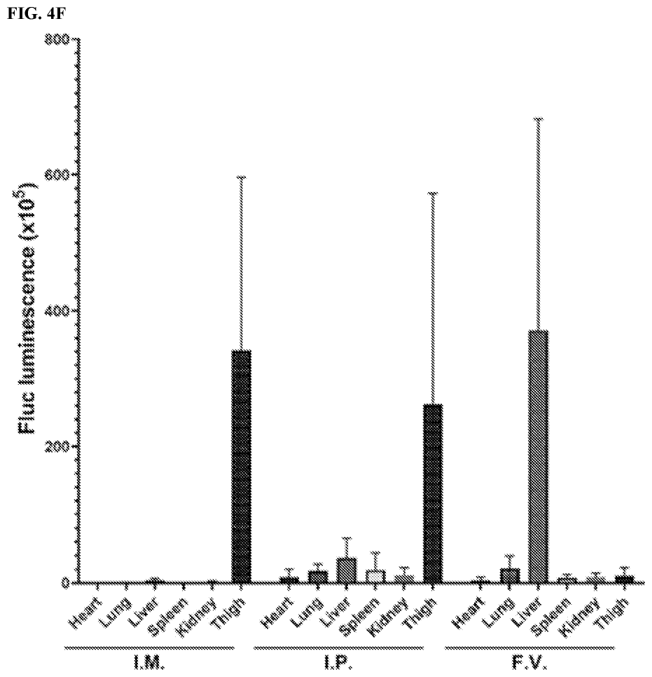

Biodistribution of LNP88 was also tested at different time points in neonatal mice via different routes of administration. FIG. 4A shows representative images of bioluminescence distribution in P2 neonatal mice treated with LNP88/mLuc over time via I.M, I.P and F.V. (e.g., facial vein) injection, and the results were quantified in FIGS. 4B-D. Major organs of neonatal mice were dissected and imaged at 55 hours post Luc mRNA/LNP injection. As shown in FIG. 4E and quantified in FIG. 4F, the routes of administration affected the biodistribution of Luc mRNA/LNP and thus lead to different expression of Luc.

### Example 4. Spike mRNA/LNP Formulations for Vaccination Against Covid 19

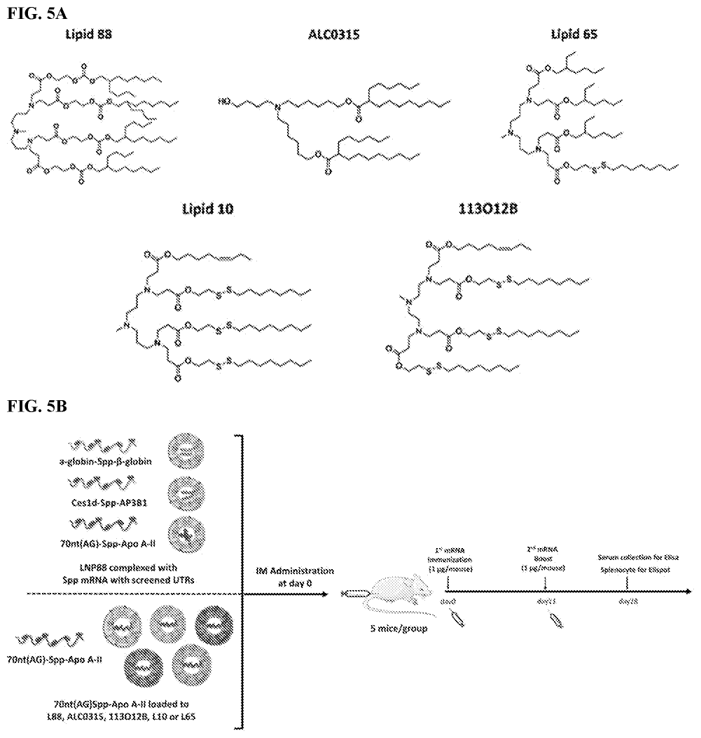

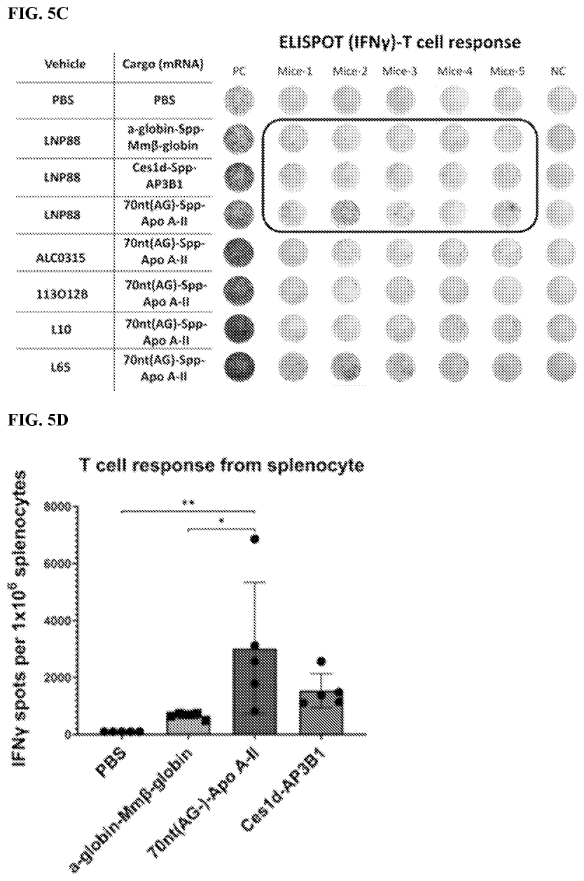

Vaccinations against Covid 19 can introduce a SARS-CoV2 antigen comprising a SARS-CoV spike protein into a subject. To improve antigen mRNA delivery and antigen expression, spike mRNA with various untranslated regions (e.g., 5′ UTR and/or 3′ UTR) were encapsulated by the LNPs and injected intramuscularly into adult mice. Five ionizable lipids, as depicted in FIG. 5A, were used in formulating the mRNA/LNPs. As depicted in FIG. 5B, the mRNA/LNPs comprise various UTRs (e.g., 5′ UTR and/or 3′ UTR). After two doses of spike mRNA/LNPs on day 0 and day 21, serum IgG titer was determined by ELISA and T cell response was measured by enzyme-linked immunosorbent spot (ELISPOT). Representative T cell response ELISpot images for the 1 μg mouse cohorts are shown in FIG. 5C and quantified in FIGS. 5D-5E. The 5′UTR-3′UTR combination 70 nt (AG)-Apo A-II resulted in the highest T cell response, as depicted in FIG. 5D, compared to a-globin-Mmb-globin and Ces1d-AP3B1. As depicted in FIG. 5E, spike mRNA comprising the 5′UTR-3′UTR combination 70 nt (AG)-Apo A-II formulated with lipid 88 and lipid 65 resulted in the highest T cell response. The effect of each UTR combination on mice IgG anti-spike antibody titer was evaluated by ELISA. As depicted in FIG. 5F, the 5′UTR-3′UTR combination Ces1d-AP3B1 resulted in the highest anti-spike IgG titer. LNPs comprising lipid 88 seemed to achieved the highest anti-spike IgG titer in mice, as shown in FIG. 5G. Significance was statistically performed using a one-way ANOVA nonparametric Brown-Forsythe test. *p<0.05, **p<0.005, ***p<0.0005.

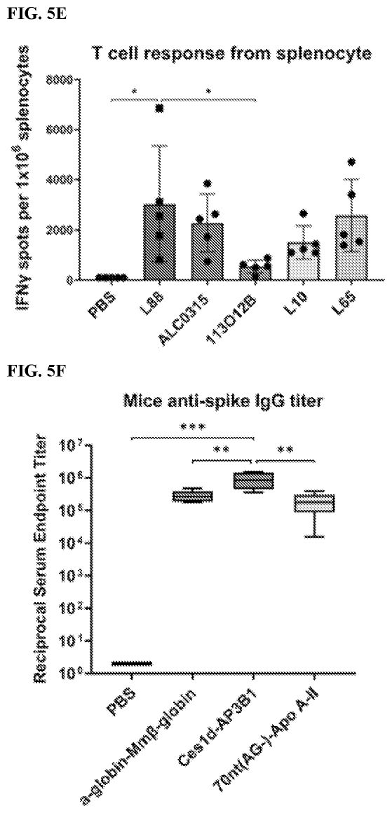

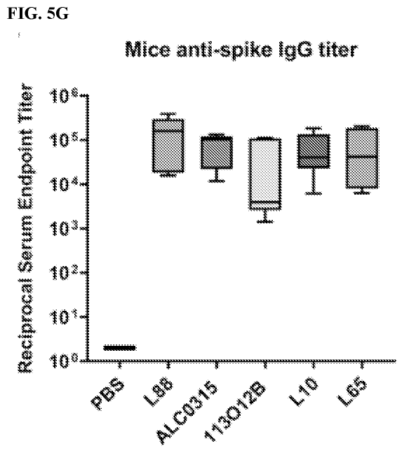

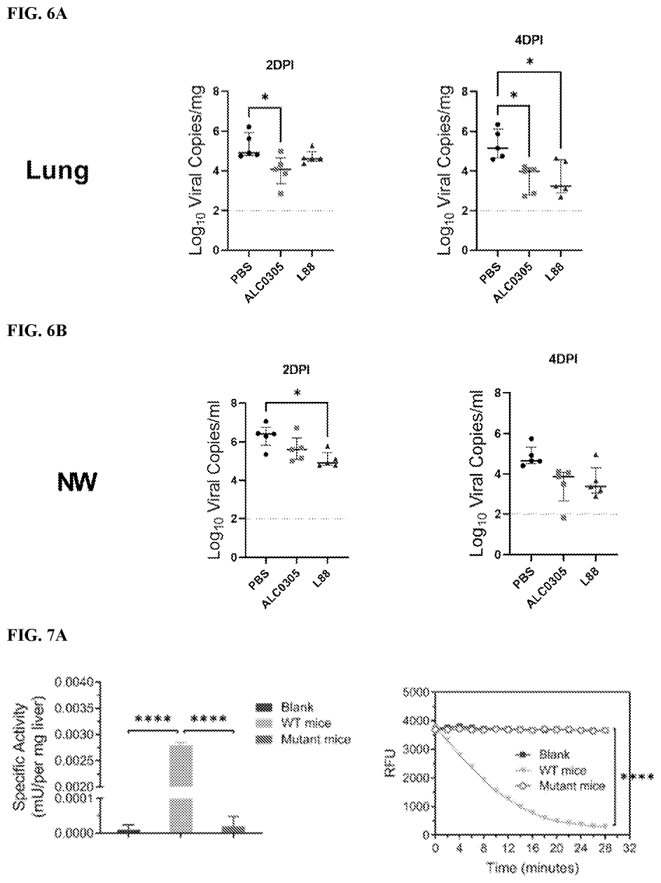

Next, Golden Syrian hamsters were vaccinated with spike mRNA/LNP comprising lipid 88. The animals were then challenged with SARS-CoV2 Omicron BQ.1, and viral genome copies in various tissues were measured 2 to 4 days post infection. FIG. 6A shows the SARS-COV-2 genome copy number in lung tissue on 2 days post infection (2 DPI) and 4 DPI. Spike mRNA/LNP comprising lipid 88 achieved lower viral genome copies in the lungs compared to control on 4 DPI. FIG. 6B shows the SARS-COV-2 genome copy number in nasal wash at 2 DPI and 4 DPI. Spike mRNA/LNP comprising lipid 88 achieved lower viral genome copies in the nasal washes compared to control on 2 DPI. Significance was statistically determined by one-way ANOVA nonparametric Brown-Forsythe test, ns, no significance, *p<0.05. Source data are provided as a Source Data file.

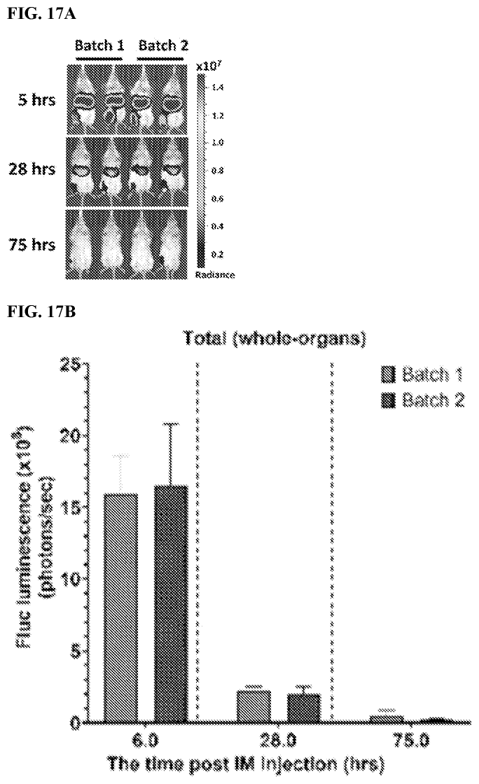

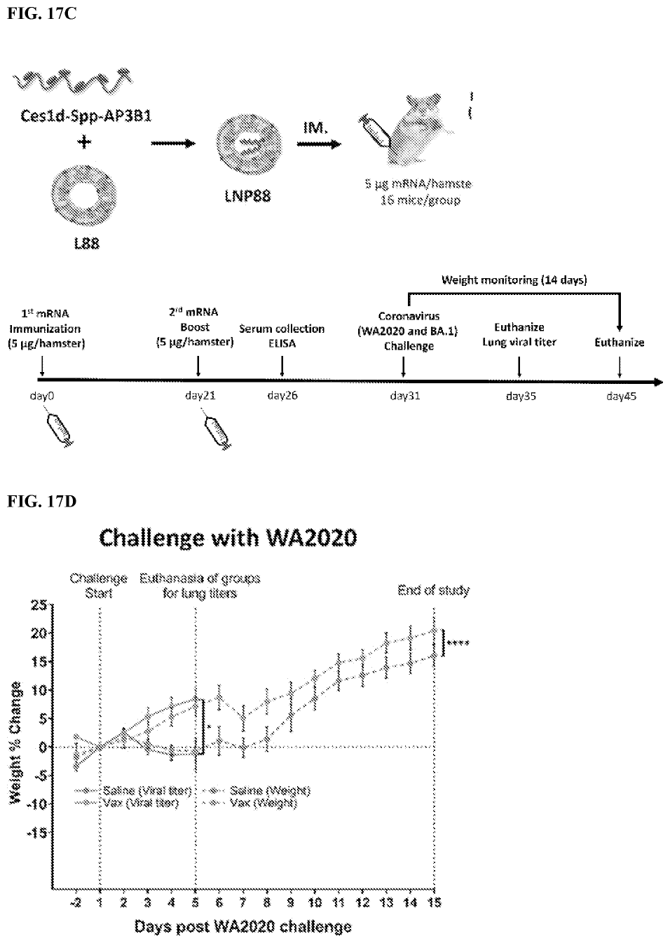

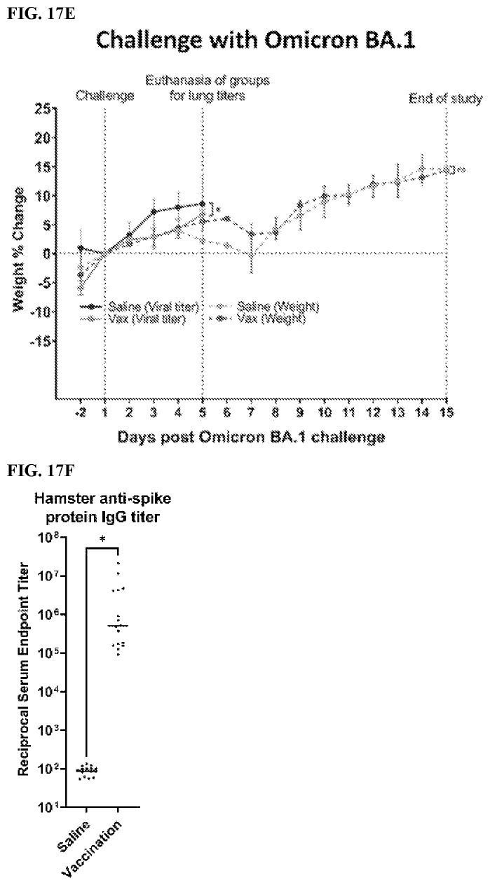

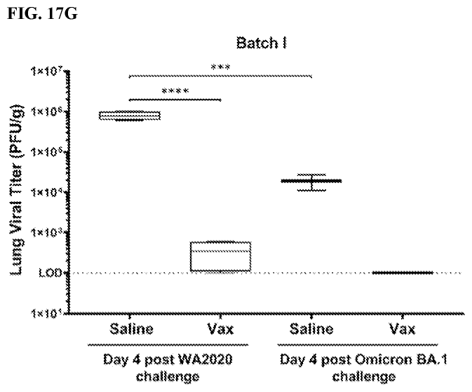

More than one batches of lipid 88 were synthesized for formulating the spike mRNA/LNPs. Each time a new batch of lipids were made, they were formulated with mLuc mRNA to confirm their delivery efficacy and consistency between batches. As depicted in FIG. 17A, similar delivery efficiency of two batches of mLuc mRNA/LNPs comprising lipid 88 was observed in mice over time (e.g., 5 hrs, 28 hrs and 75 hrs). FIG. 17B shows the quantification of f.Luc expression levels in radiance (photons/sec/cm²/sr) in mice over time.

Next spike mRNA/LNPs comprising lipid 88 was also confirmed to protect Golden Syrian hamsters from the other SARS-CoV2 variants (e.g., WA1/2020, Omicron BA.1). Spike mRNA comprising the 5′ UTR-3′UTR combination Ces1d-AP3B1 were formulated with the ionizable lipids provided herein (e.g., lipid 88). And the spike mRNA/LNPs were characterized as described herein. FIG. 17C shows a schematic of the hamster immunization timeline, including two doses Ces1d-Spp-Ap3B1 mRNA/LNP88 prior to serum collection and SARS-COV-2 challenge. Each hamster was immunized with a 5 μg dose of spike mRNA/LNPs comprising lipid 88 (n=4 each for lung viral titer; n=8 each for weight monitoring) or mock-immunized with saline (saline) (n=4 each for lung viral titer; n=8 each for weight monitoring), then challenged with 4.0×10⁴ total plaque-forming units (PFU) of WA1/2020 and 1.3×10⁴ total PFU of Omicron BA.1 under the intranasal route. Lung viral titer was evaluated by lung titer plaque assays. Reduced viral loads in hamsters after WA 1/2020 and Omicron BA. 1 challenge were observed in the vaccinated group, as depicted in FIGS. 17D-17E, and FIG. 17G. FIG. 17G shows a comparison of lung viral titers in WA1/2020 or Omicron BA.1-infected hamsters with or without immunization. Batch I and Batch II were set up to confirm reproducibility. Batch I cohorts include hamsters vaccinated with saline or eSV1 (5 μg/hamster) followed by infection with WA1/2020 (4.0×10⁴ PFU/hamster, n=4) and hamsters vaccinated with saline or eSV1 (5 μg/hamster) followed by infection with Omicron BA.1 (1.3×10⁴ PFU/hamster, n=2). Batch I cohorts hamsters vaccinated with saline or eSV1 (5 μg/hamster) followed by infection with WA1/2020 (4.0×10⁴ PFU/hamster, n=5) and hamsters vaccinated with saline or eSV1 (5 μg/hamster) followed by infection with Omicron BA.1 (2.3×10⁴ PFU/hamster, n=5). LOD: 100 pfu/g lung.

FIGS. 17D-17E depict body weight change (%) of hamsters following WA1/2020 challenge (4.0×10⁴ PFU/hamster, n=8) and Omicron BA.1 challenge (1.3×10⁴ PFU/hamster, n=2). Vaccinated mice had more weight gain compared to unvaccinated mice after WA2020 challenge. High serum titers of SARS-COV-2 Spike-specific IgG were observed in vaccinated hamsters one week post-booster (n=16 per group) as depicted in FIG. 17F.

Significance for FIGS. 17D-17G was statistically determined by one-way ANOVA nonparametric Brown-Forsythe test, ns, no significance, *p<0.05, **p<0.005, ***p<0.0005, ****p<0.00005.

In summary, vaccinating mice with spike mRNA/LNPs comprising the ionizable lipids (e.g. lipid 88) achieved improved T cell response, higher serum anti-spike IgG titers, and lower viral load post infection by SARS-CoV2 variants of concern (e.g., WA1/2020, Omicro BA.1, and Omicro BQ.1).

### Example 5. Ugt1a1 mRNA/LNPs Rescue the Symptoms of Cigler-Najjar (CN) Syndrome in Ugt1a1−/− Neonatal Mice

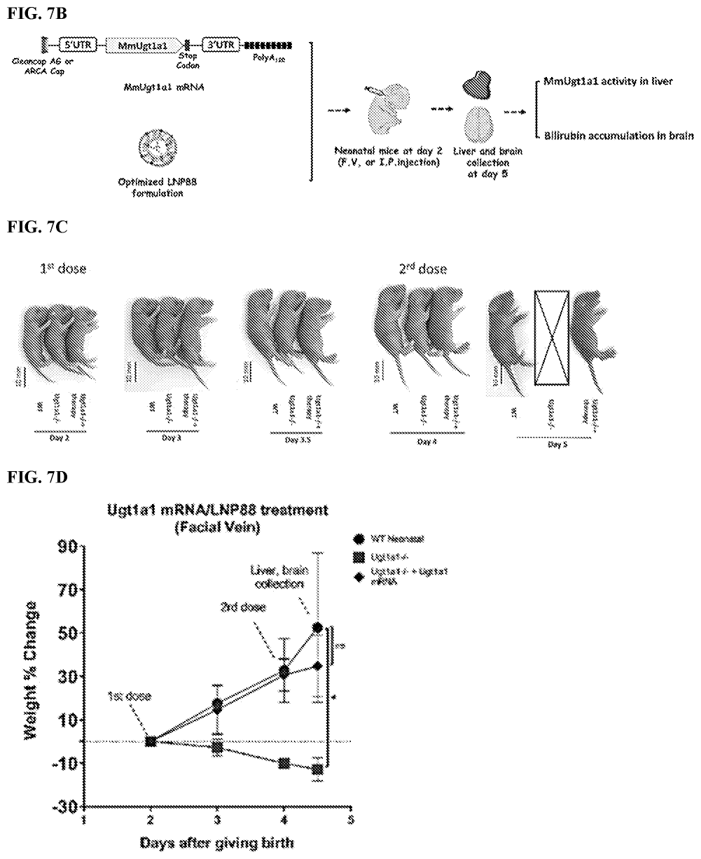

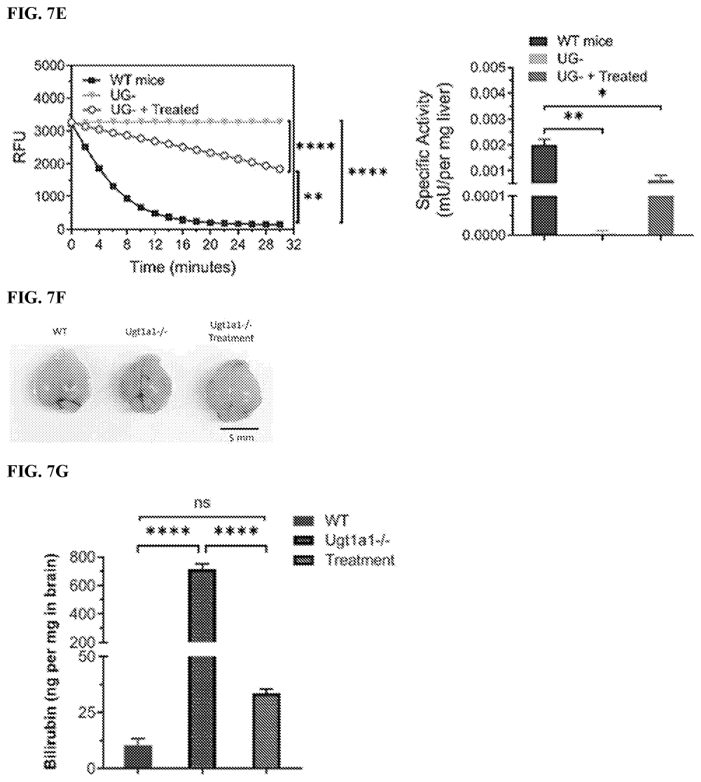

Crigler-Najjar syndrome is a rare autosomal recessive disorder of bilirubin conjugation characterized by severe unconjugated hyperbilirubinemia that can result in bilirubin-induced neurologic dysfunction (BIND). Crigler-Najjar syndrome is caused by an absence or profoundly decreased level of the enzyme UDP-glucuronosyltransferase due to a genetic defect in the UGT1A1 gene. The LNPs provided herein can deliver the UGT1A1 mRNA to a subject thus improve symptoms of CN syndrome, for example, improve liver enzyme activity and reduce bilirubin accumulation in the brain. As shown in FIG. 7B, Ug1a1 mRNA/LNPs comprising lipid 88 were injected via various routes (e.g., intrapertonally or through facial vein) into Ug1a1−/− neonatal pups on pos-natal day 2. More than one dose of the Ug1a1 mRNA/LNPs can be administered. In some cases, a second dose of the Ug1a1 mRNA/LNPs was administered on post-natal day 4. Then the liver and brains of the pups were collected on post-natal day 5. Ug1a1 activity was measured in liver lysate and the accumulation of bilirubin was measured in the brain. The Ug1a1 mRNA/LNP treatment successfully rescued the enzymatic activity of Ug1a1 in the liver and reduced bilirubin accumulation in the brain.

First, we confirmed the level of specific activity of Ug1a1 in liver lysate of WT and Ug1a1−/− mutant mice using a fluorescence-based assay. As depicted in FIG. 7A, mutant mice showed no Ug1a1 activity in liver lysate. FIG. 7C shows representative images of neonatal growth over time until the Ugt1a1−/− mutant expired. FIG. 7D shows the weight change (%) in WT, Ugt1a1−/− mutant and mUg1a1/LNP88 treated group. Ug1a1 mRNA/LNPs treated pups maintained their weight similar to WT pups. FIG. 7E shows the analysis of Ug1a1 activity in liver lysate from WT, Ug1a1−/− mutant and mUg1a1/LNP88 treated group. Ug1a1 mRNA/LNPs successfully restored the specific activity of Ug1a1 in the liver lysate of mutant pups. FIG. 7F depicts brain sizes of WT, treated, and untreated Ug1a1−/− pups on post-natal day 5. The Ug1a1 mRNA/LNPs treatment successfully rescued the brain size of Ug1a1−/− pups. FIG. 7G depicts a bar graph quantifying the amount of bilirubin in 5-day old neonatal mice brains. Ug1a1 mRNA/LNPs treatment significantly reduced the amount of bilirubin in Ug1a1−/− pups.

### Example 6 ABE8e/sgRNA/LNPs Delivery Resulted in Loss of Function of Angptl 3 and Improved Lipid Profile

The LNPs of the present disclosure can comprise more than one payload including mRNA encoding a protein and small RNA molecules (e.g., sgRNAs). For example, mRNA encoding an adenine base editor (e.g., ABE8e) and sgRNAs were formulated with the ionizable lipids provided herein, together with cholesterol and helper lipids. Delivery of ABE8e mRNA and sgRNAs resulted in expression of functional ABE8e enzyme and successful base editing. Loss of function (LOF) variants of Angptl 3 are naturally occurring in the human population with no known associated complications. Individuals with these mutations have shown reduced levels of plasma low density lipoprotein cholesterol (LDL-C) and triglycerides (TG), as well as protection against coronary artery disease (CAD). CRISPR Cas nucleases can be used to induce LOF of Angptl 3 for protection against liver diseases, dyslipidemia, and CAD. Although this approach was successful, the use of Cas to induce double strand breaks may lead to genomic translocations or signaling cascades leading to cellular apoptosis. To overcome these limitations, here we adapted the base editing approach. We demonstrated that the delivery of mRNA encoding ABE8e and a Angptl 3-targeting sgRNAs using lipid 88 formulations achieved more than 60% of Angptl 3 knockdown.

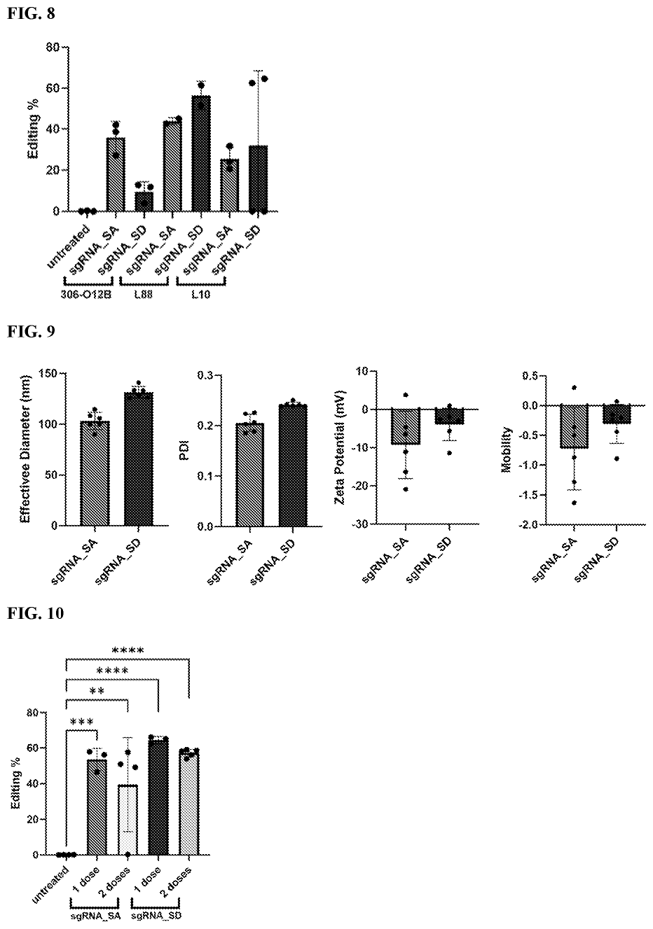

ABE8e mRNA/sgRNA/LNPs (e.g., LNPs formulated with various ionizable lipids, ABE8e mRNA, and sgRNAs) resulted in increased base editing. sgRNAs compatible with ABE8e were designed for both the splicing acceptor and donor sites for splice disruptions in Exon 6 of Angltl 3. As shown in FIG. 8, lipid 88 showed the highest transfection and editing efficiency (almost 60%). Sizes, PDI, zeta potential, and mobility of all ABE8e mRNA/sgRNA/LNPs were characterized, and representative data for LNPs comprising lipid 88 are shown in FIG. 9.

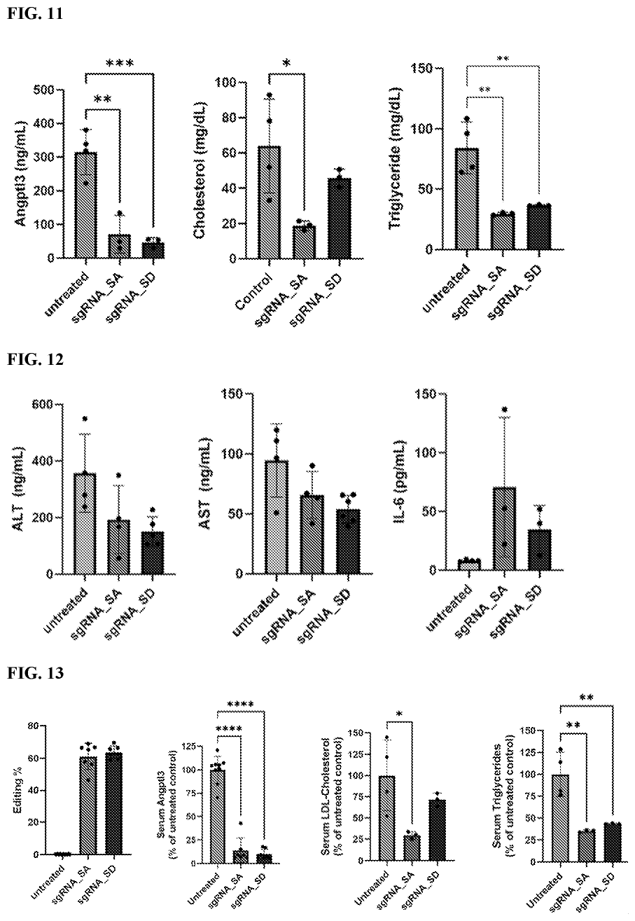

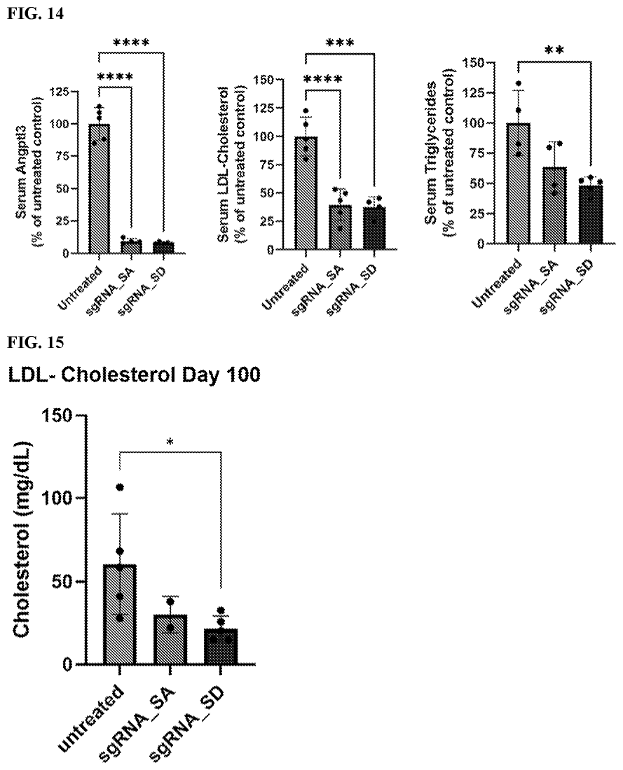

Next LNPs (e.g., ABE8e mRNA/sgRNA/LNPs comprising lipid 88) were injected to rodents. In some cases, one dose of 3 mg/kg LNPs was administered. In some cases, 2 doses of 3 mg/kg LNPs were administered 48 hours apart. As depicted in FIG. 10, percent of base editing was higher in the single dose group. Significance measured with one way ANOVA. *P<0.05 **P<0.01 ***P<0.001 ****P<0.0001. Seven days post administration of the LNPs, blood samples were collected from treated animals, and various biomarkers were measured, including Angptl3, cholesterol, triglyceride, ALT, AST, and IL-6. As depicted in FIG. 11, significant decrease in serum levels of Angptl3, cholesterol, and triglyceride were observed 7 days after single administration of LNPs. Significance measured with one way ANOVA *P<0.05 **P<0.01 ***P<0.001 ****P<0.0001. No significant toxicity of LNPs was observed 48 hours post administration. As depicted in FIG. 12, there was no significant changes in alanine transaminase (ALT), aspartate transaminase (AST), and Interluekin-6 (IL-6). Significance measured with one way ANOVA *P<0.05 **P<0.01 ***P<0.001 ****P<0.0001. Percentage of base editing, levels of ANGPTL3, LDL-C, and Triglycerides were reduced on Day 7 and Day 30 post injection of LNPs (e.g., ABE8e mRNA/sgRNA/LNPs comprising lipid 88), as depicted in FIGS. 13-14. Significance measured by one-way ANOVA. FIG. 15 depicts the reduction of LDL-C 100 days post injection of LNPs (e.g., ABE8e mRNA/sgRNA/LNPs comprising lipid 88). Significance measured by one-way ANOVA.

### Example 7. In Vivo UTRs Optimization for Targeted Delivery of mRNA/LNPs

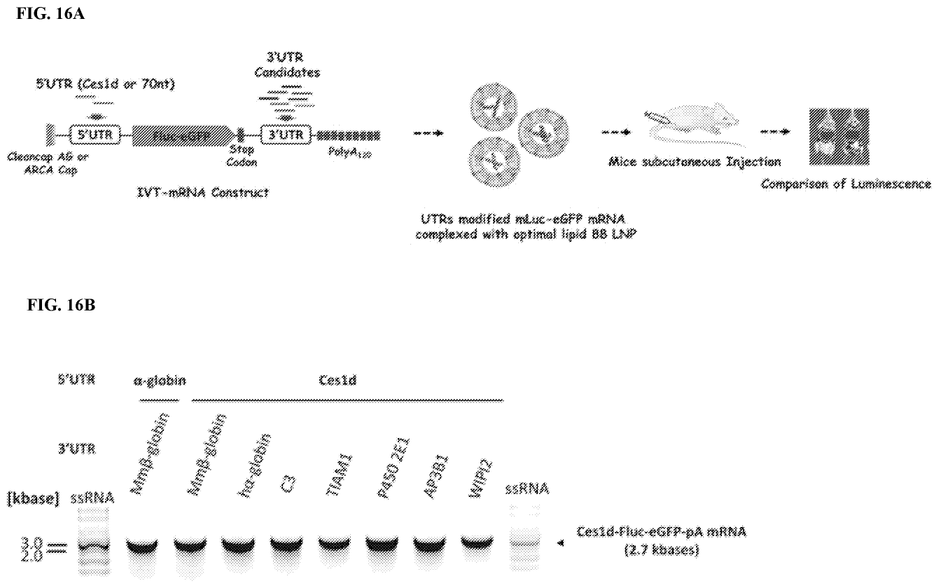

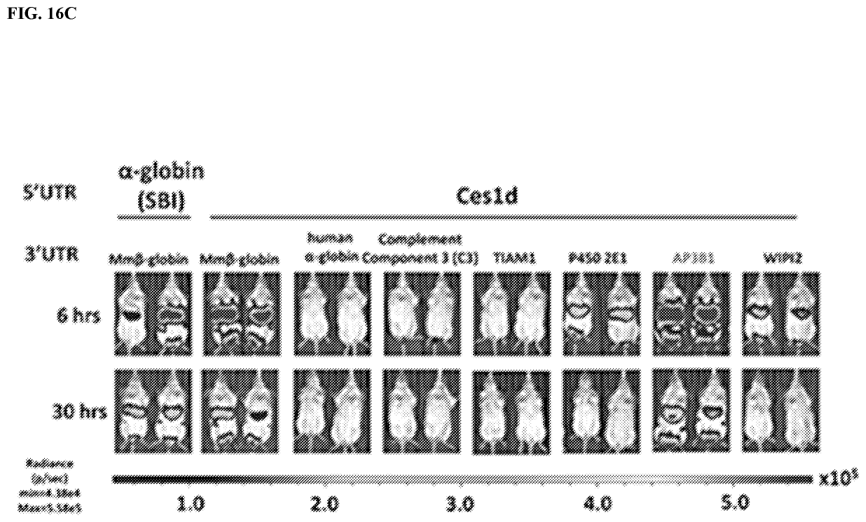

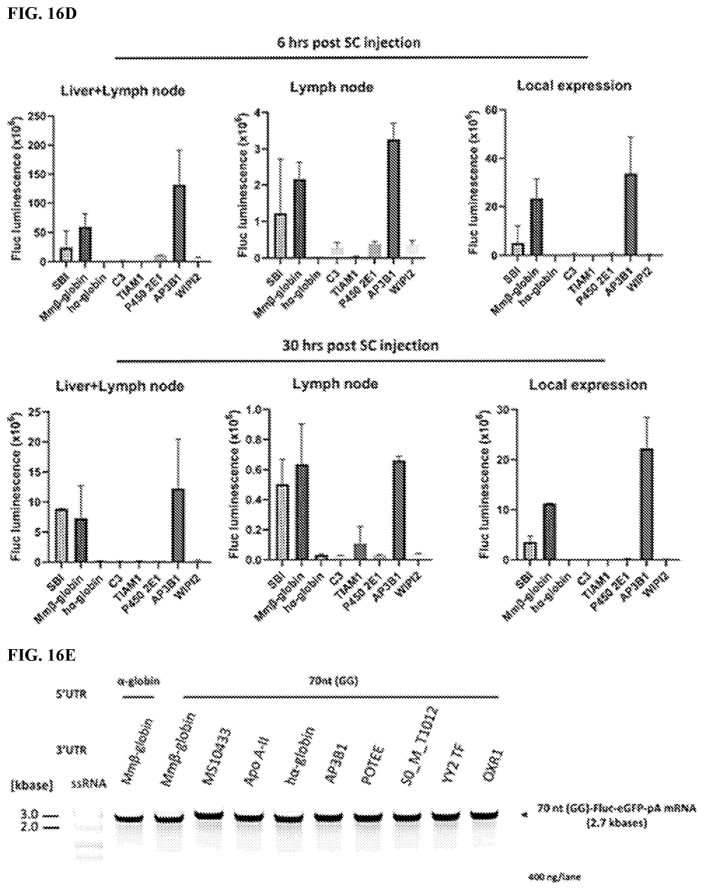

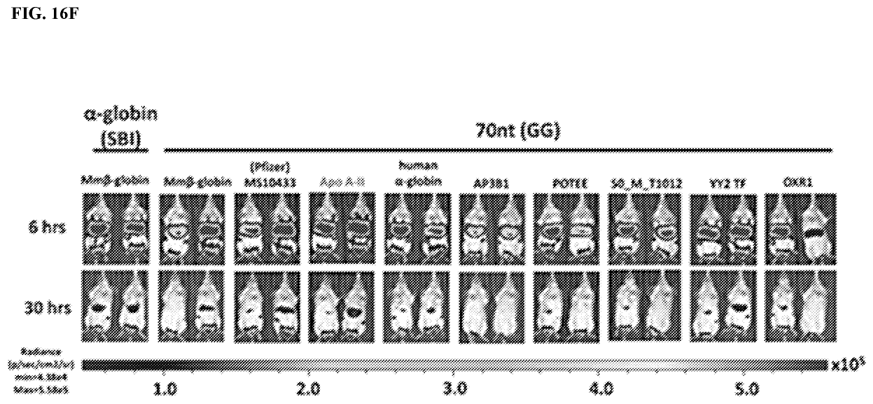

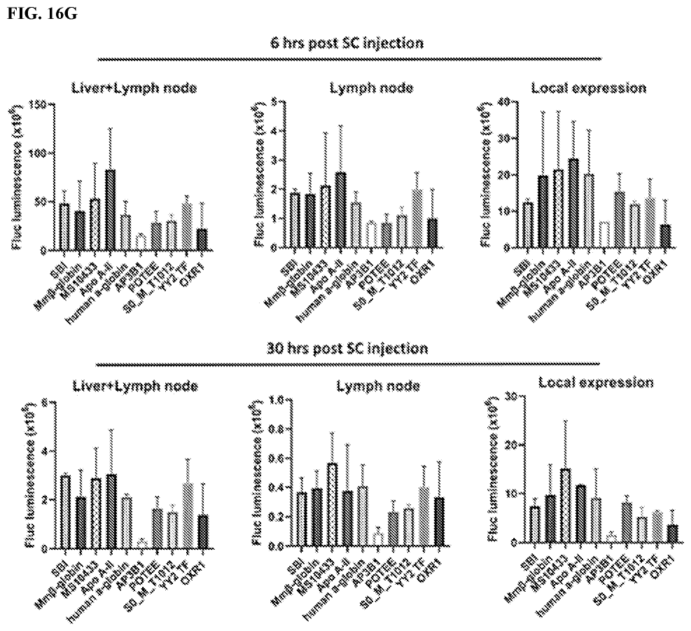

Changing the untranslated regions of the mRNA (e.g., 5′ UTR and/or 3′ UTR) can improve delivery and expression of mRNA/LNPs. 5′ UTR and 3′ UTR candidates were incorporated into mLuc-eGFP mRNA and formulated with the ionizable lipids provided herein (e.g., lipid 88). UTR-modified mLuc-eGFP mRNA/LNPs comprising lipid 88 were injected subcutaneously into mice for in vivo testing, as depicted in the experimental scheme in FIG. 16.

First the 5′ UTR Ces1d was adopted in the mFLuc-eGFP mRNA/LNP, and various 3′ UTRs were assessed. FIG. 16B depicts that the mFLuc-eGFP mRNAs comprising 5′ UTR Ces1d each also contained a 3′UTR selected from the group Mmβ-globin, ha-globin, C3, TIAM1, P450 2E1, AP3B1 and WIPI2. FIG. 16C shows the in vivo bioluminescence levels from 6 hrs to 30 hrs following subcutaneous injection of mLuc-eGFP/LNP comprising lipid 88 with Ces1d as the 5′UTR. Scales represent radiance in photons/sec/cm²/sr. FIG. 16D shows the quantification of Firefly luciferase (FLuc) expression over time localized in various organ systems (liver, lymph node, and local injection site). ±SD (n=2). At 6 hours and 30 hours post SC injection, mFLuc-eGFP mRNA/LNPs comprising the 3′ UTR Mmβ-globin and AP3B1 showed enhanced expression at all locations imaged.

First the 5′ UTR 70 nt (GG) was adopted in the mFLuc-eGFP mRNA/LNP, and various 3′ UTRs were assessed. FIG. 16E depicts that the mLuc-eGFP mRNAs comprising the 5′ UTR 70 nt (GG) each also contained a 3′UTR selected from the group Mmβ-globin, MS10433, Apo A-II, ha-globin, AP3B1, POTEE, S0_M_T1012, YY2 TF, and OXR1. FIG. 16F shows the bioluminescence measurements from 6 hrs to 30 hrs following subcutaneous injection of mLuc-eGFP/LNP with 70 nt as the 5′UTR. Scale represents radiance in photons/sec/cm²/sr. FIG. 16G shows the quantification of FLuc expression over time at the location of bioluminescence distribution in mice (liver, lymph node and local). ±SD (n=2). At 6 hours and 30 hours post SC injection, mFLuc-eGFP mRNA/LNPs comprising the 3′ UTR Mmβ-globin, MS10433, Apo A-II, ha-globin, POTEE, S0_M_T1012, YY2 TF, and OXR1 showed enhanced expression at all locations imaged.

### OTHER EMBODIMENTS

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A composition, comprising: a pharmaceutical agent and a lipid composition; wherein the pharmaceutical agent is assembled with the lipid composition; and the lipid composition comprises a lipidoid having structural Formula (I): 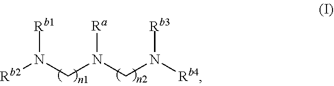
or a pharmaceutically acceptable salt thereof;
wherein:
R^{a} is a substituted or unsubstituted C₁-C₆ alkyl or C₂-C₆ hydroxyalkyl;
n1 and n2 are each independently 1, 2, 3, or 4; and
R^{b1}, R^{b2}, R^{b3} and R^{b4} are each independently 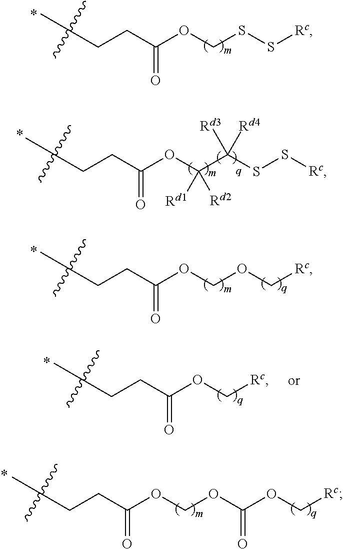
* indicates the point of attachment to N;
each R^{c} is independently an alkyl or an alkenyl;
R^{d1}, R^{d2}, R^{d3} and R^{d4} are each independently H or C₁-C₄ alkyl, wherein at least one of R^{d1}, R^{d2}, R^{d3} and R^{d4} is not H;
each m is independently an integer from 1-10; and
each q is independently an integer from 1-10.

2. The composition of claim 1, wherein R^{a} is C₁-C₄ alkyl.

3. The composition of claim 1, wherein R^{a} is C₂-C₅ hydroxyalkyl.

4. The composition of claim 1, wherein n1 is 1, 2 or 3; and n2 is 1, 2 or 3.

5.-9. (canceled)

10. The composition of claim 1, wherein R^{b1}, R^{b2}, R^{b3} and R^{b4} are each independently

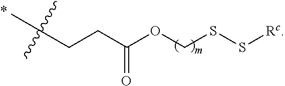

11. (canceled)

12. The composition of claim 1, wherein R^{b1}, R^{b2}, R^{b3} and R^{b4} are each independently

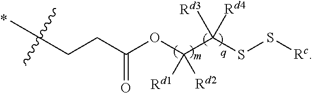

13. The composition of claim 1, wherein R^{b1}, R^{b2}, R^{b3} and R^{b4} are each independently

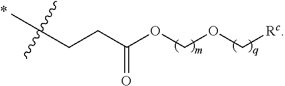

14. The composition of claim 1, wherein R^{b1}, R^{b2}, R^{b3} and R^{b4} are each independently

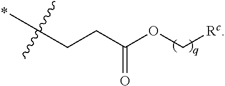

15. The composition of claim 1, wherein R^{b1}, R^{b2}, R^{b3} and R^{b4} are each independently

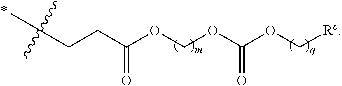

16. The composition of claim 1, wherein each R^{c} is independently C₄-C₁₆ alkyl or C₄-C₁₆ alkenyl.

17. (canceled)

18. The composition of claim 1, wherein each m is independently 1, 2, or 3.

19. (canceled)

20. (canceled)

21. The composition of claim 1, wherein each q is independently 1, 2, 3, or 4.

22. (canceled)

23. (canceled)

24. The composition of claim 1, wherein the lipidoid of Formula (I), or the pharmaceutically acceptable salt thereof, is a lipidoid having structural Formula (II_{A}), (II_{B}), (II_{C}), (II_{D}), (II_{E}), (II_{F}), (II_{G}), or (II_{H}): 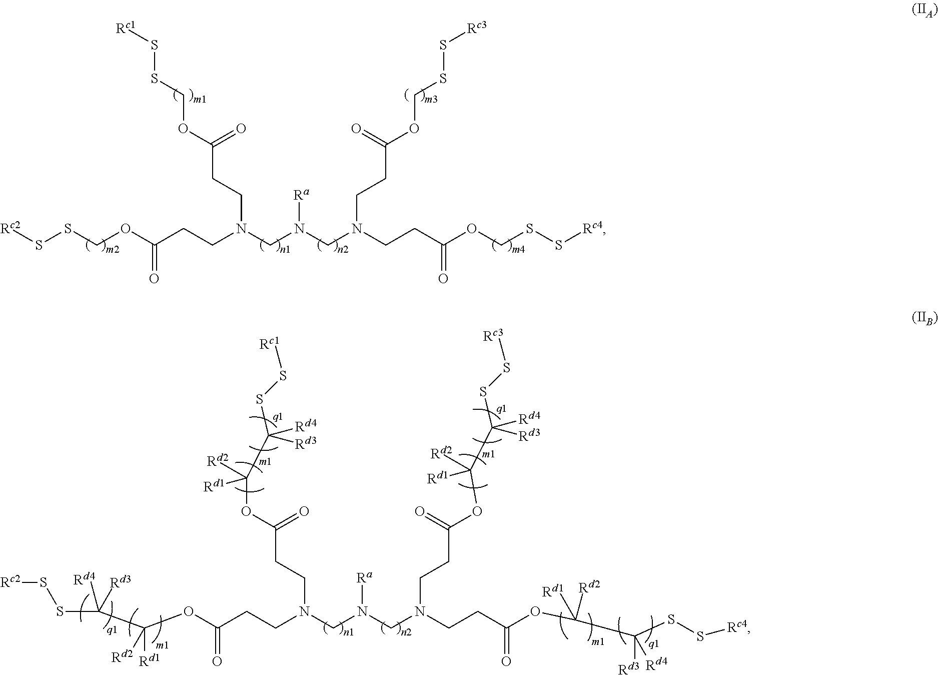 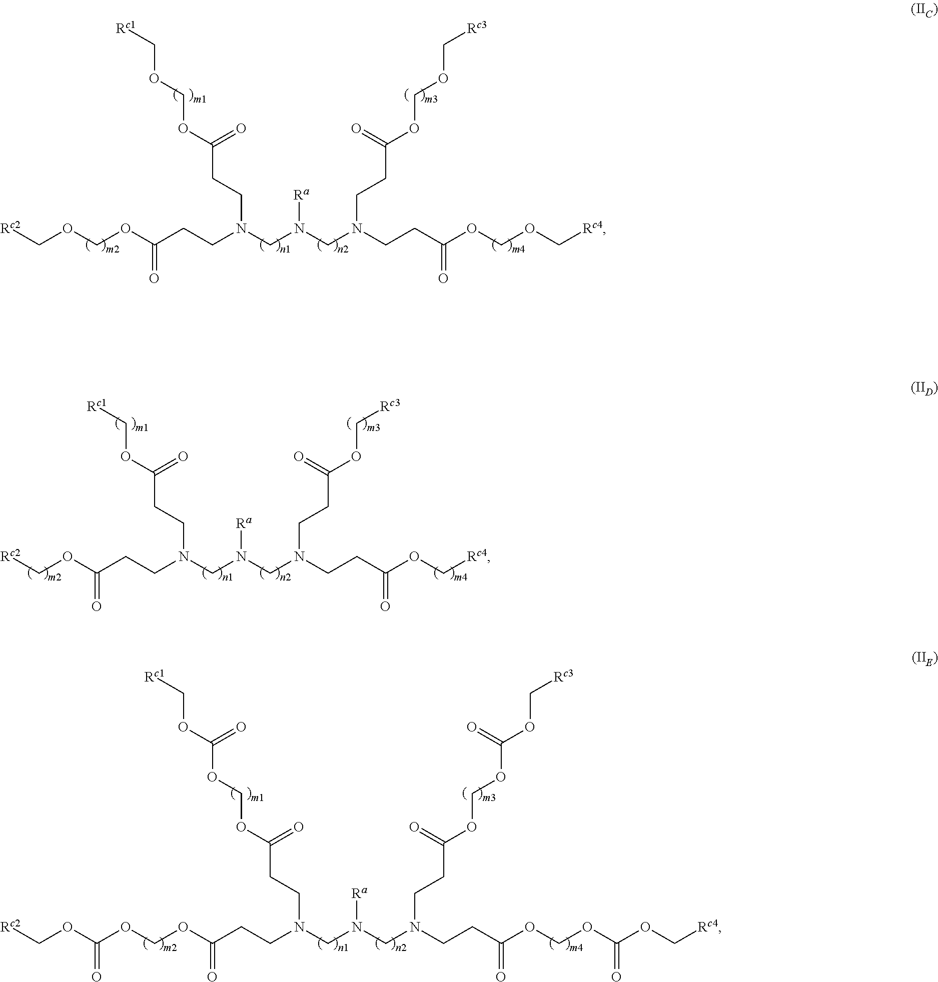 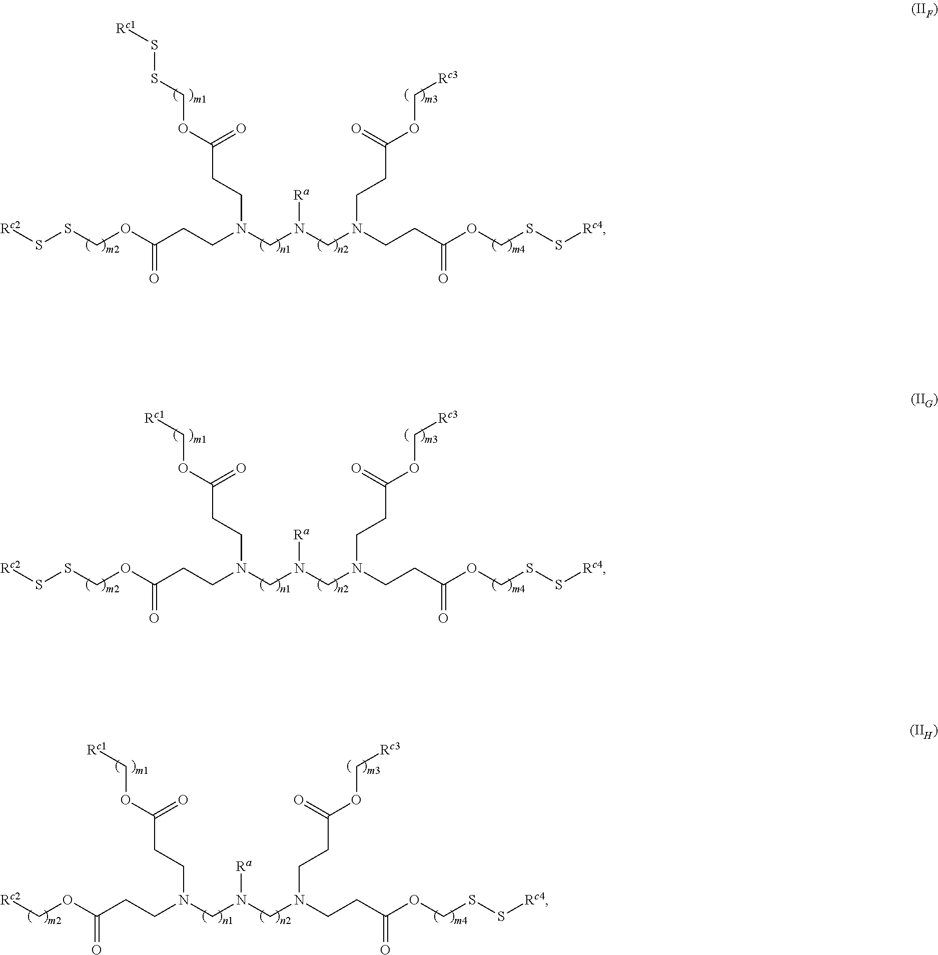
or a pharmaceutically acceptable salt thereof, wherein:
n1 and n2 are each independently 1, 2, 3, or 4;
m1, m2, m3, and m4, when present, are each independently 1, 2, or 3;
q1, q2, q3 and q4, when present, are each independently 1, 2, 3, or 4; and
R^{c1}, R^{c2}, R^{c3} and R^{c4}, when present, are each independently C₄-C₂₀ alkyl or C₄-C₂₀ alkenyl.

25. The composition of claim 1, wherein the lipidoid is selected from the group consisting of: 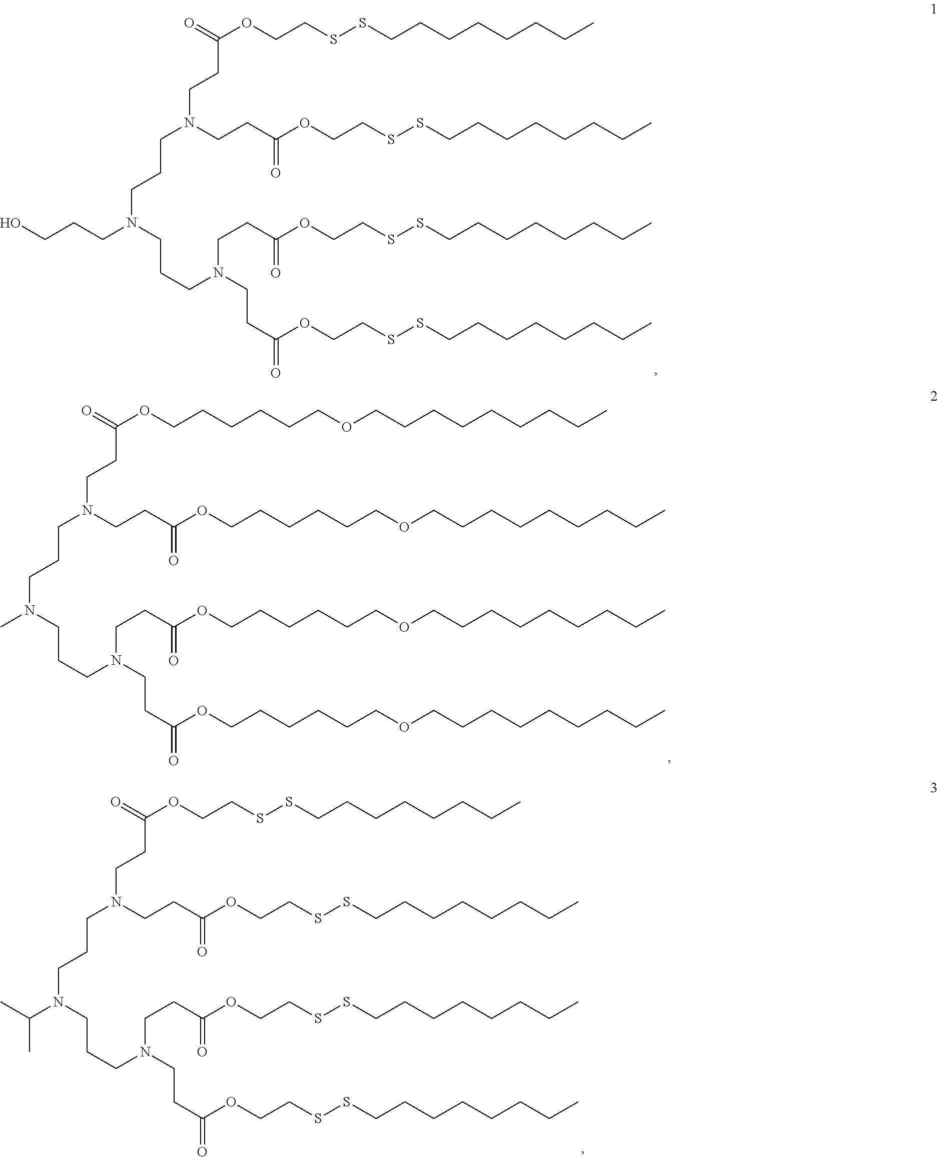 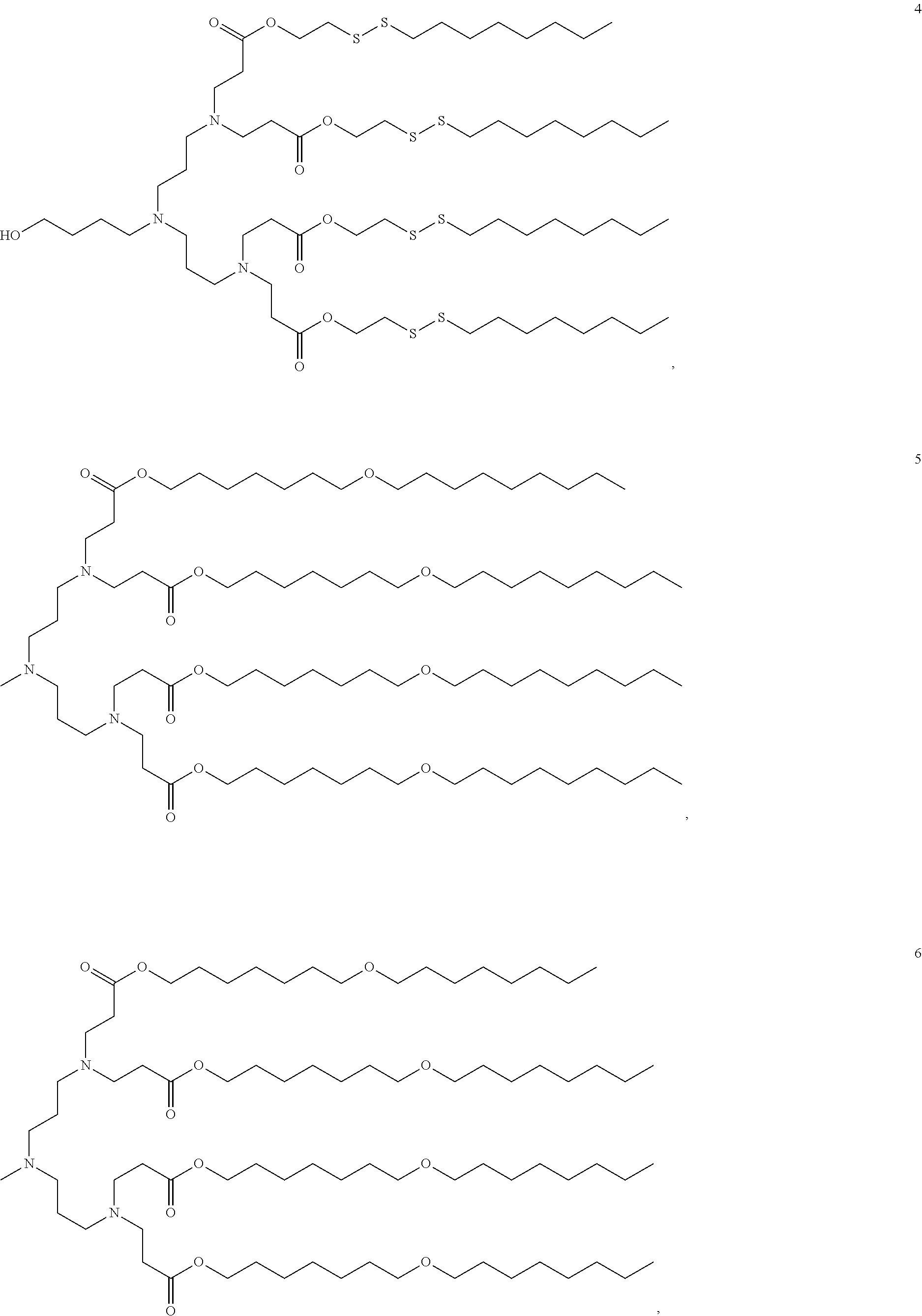 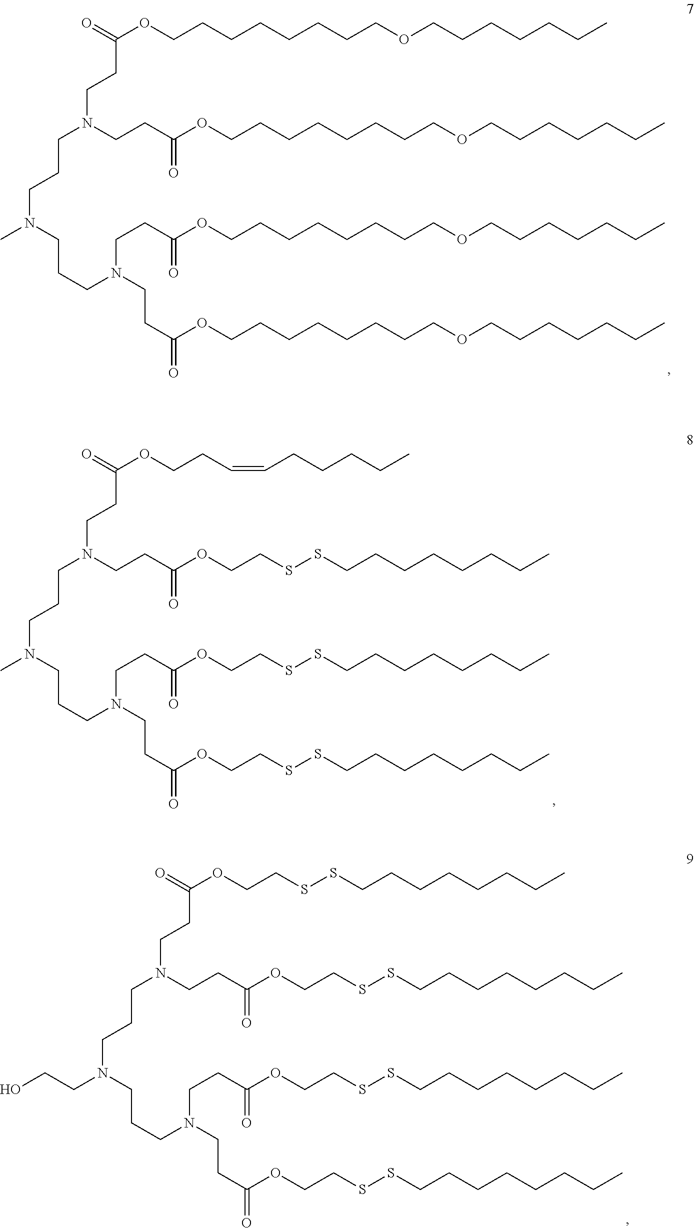 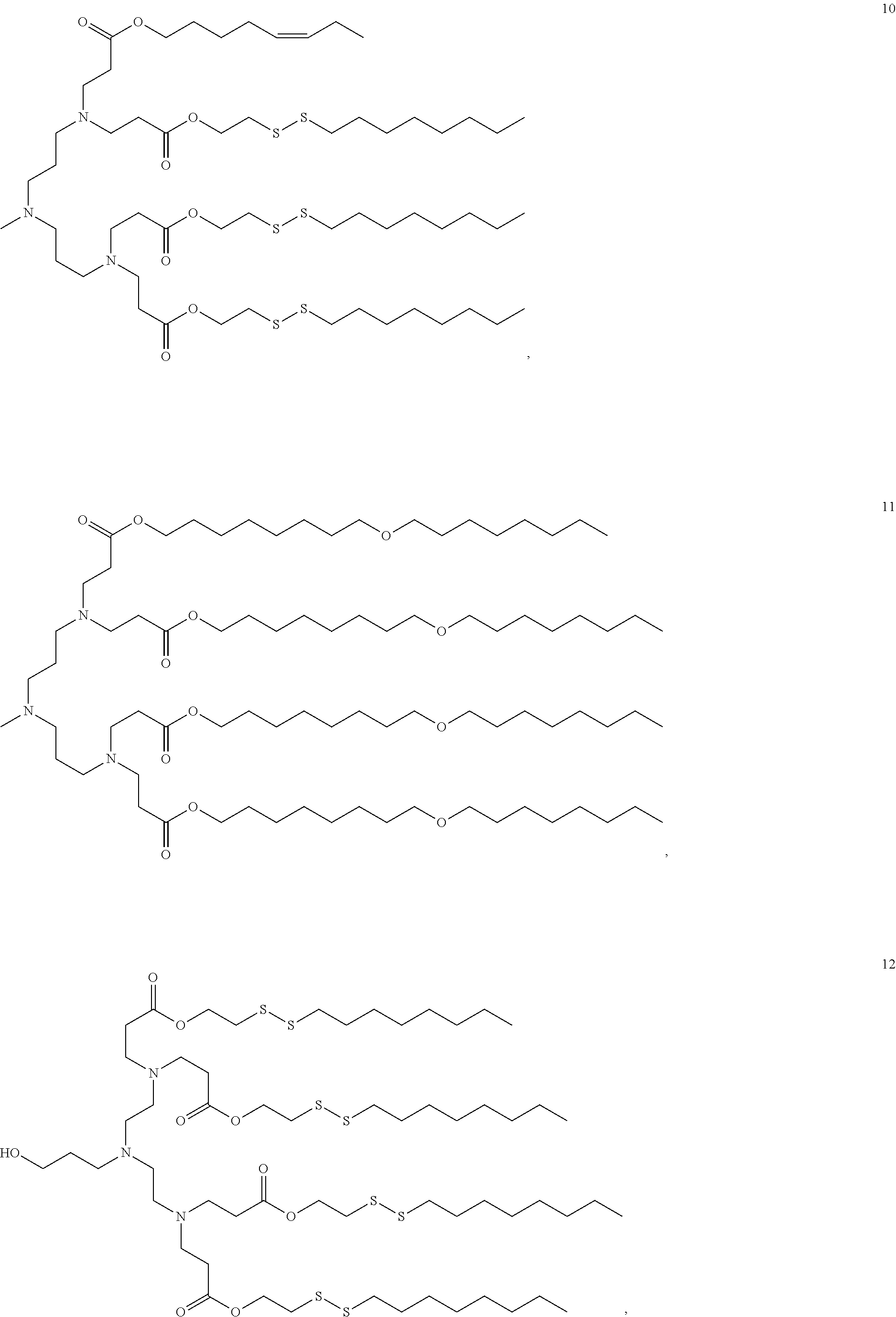 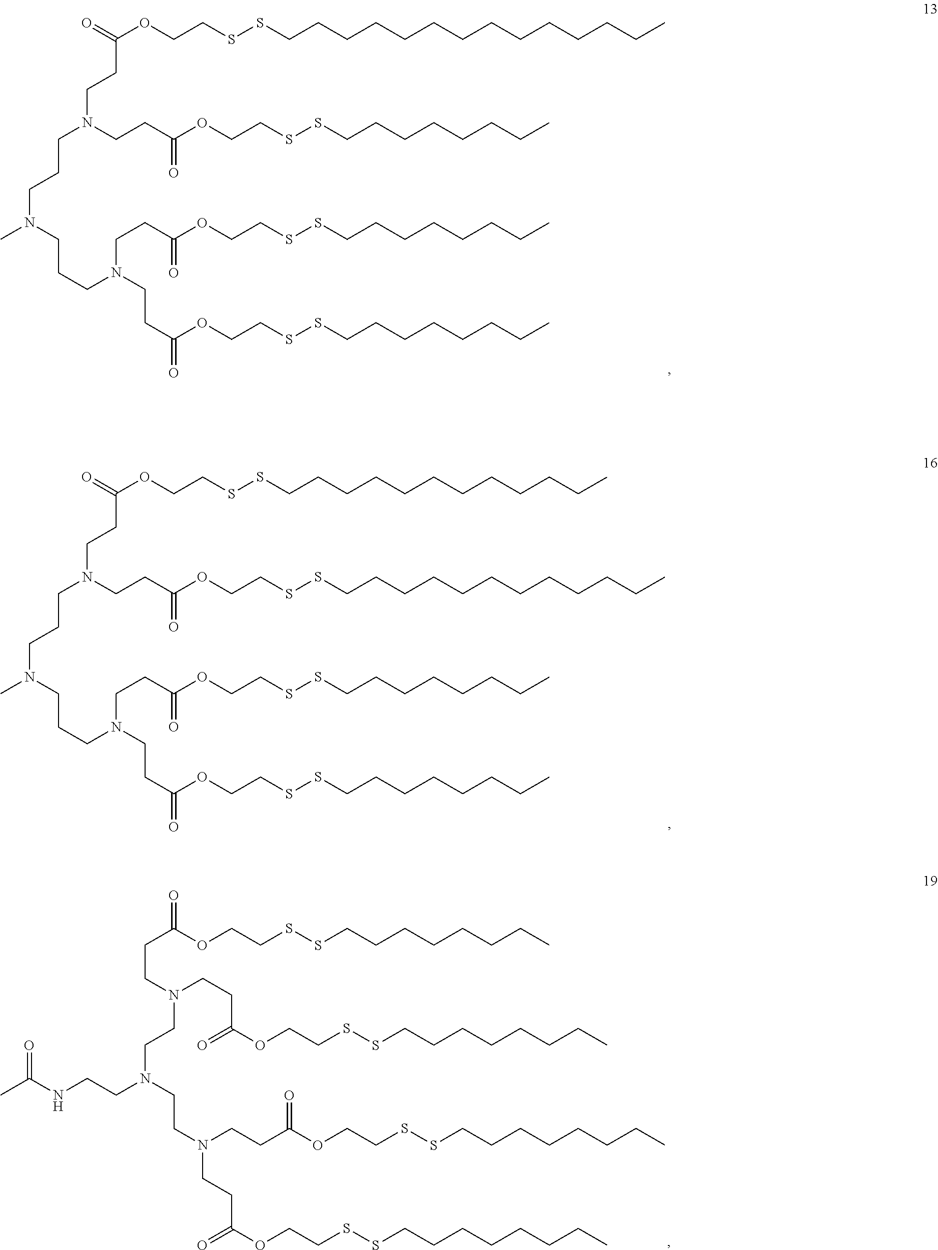 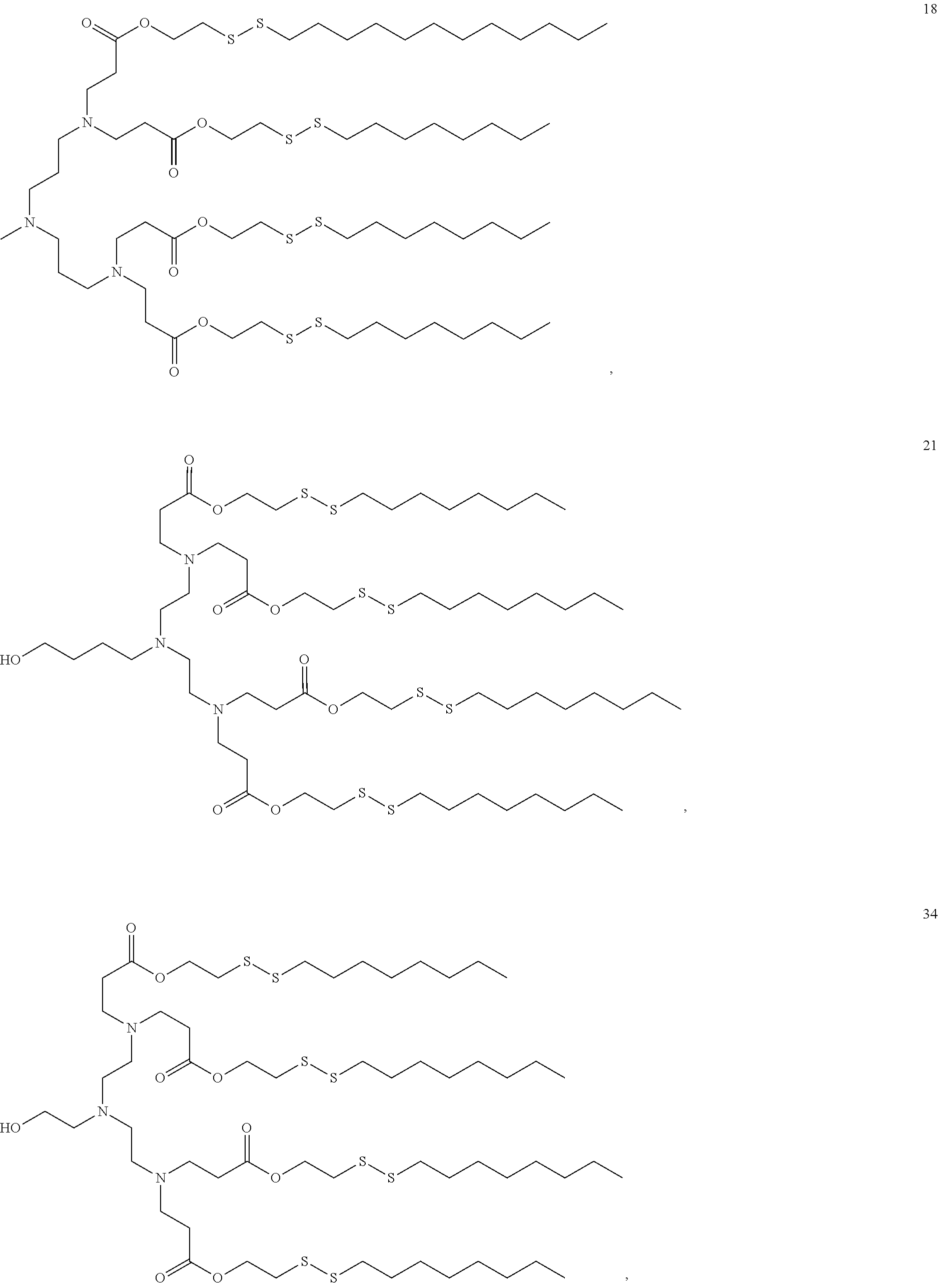 [SMILES: CCCCCCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCC ; CCCCCCCCSSCCOC(=O)CCN(CCC(=O)OCCSSCCCCCCCC)CCN(CCCCO)CCN(CCC(=O)OCCSSCCCCCCCC)CCC(=O)OCCSSCCCCCCCC ; CCCCCCCCSSCCOC(=O)CCN(CCC(=O)OCCSSCCCCCCCC)CCN(CCO)CCN(CCC(=O)OCCSSCCCCCCCC)CCC(=O)OCCSSCCCCCCCC] 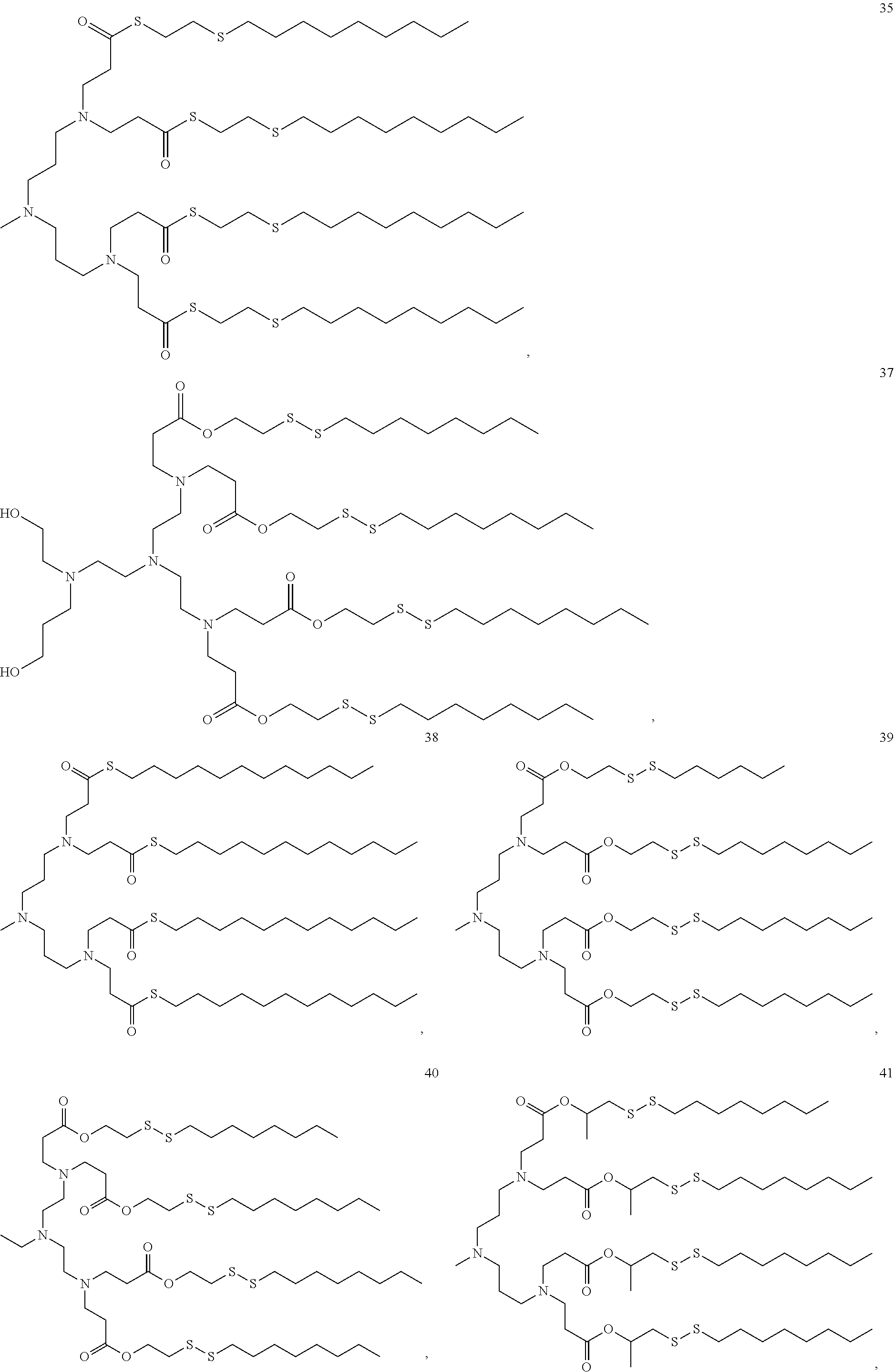 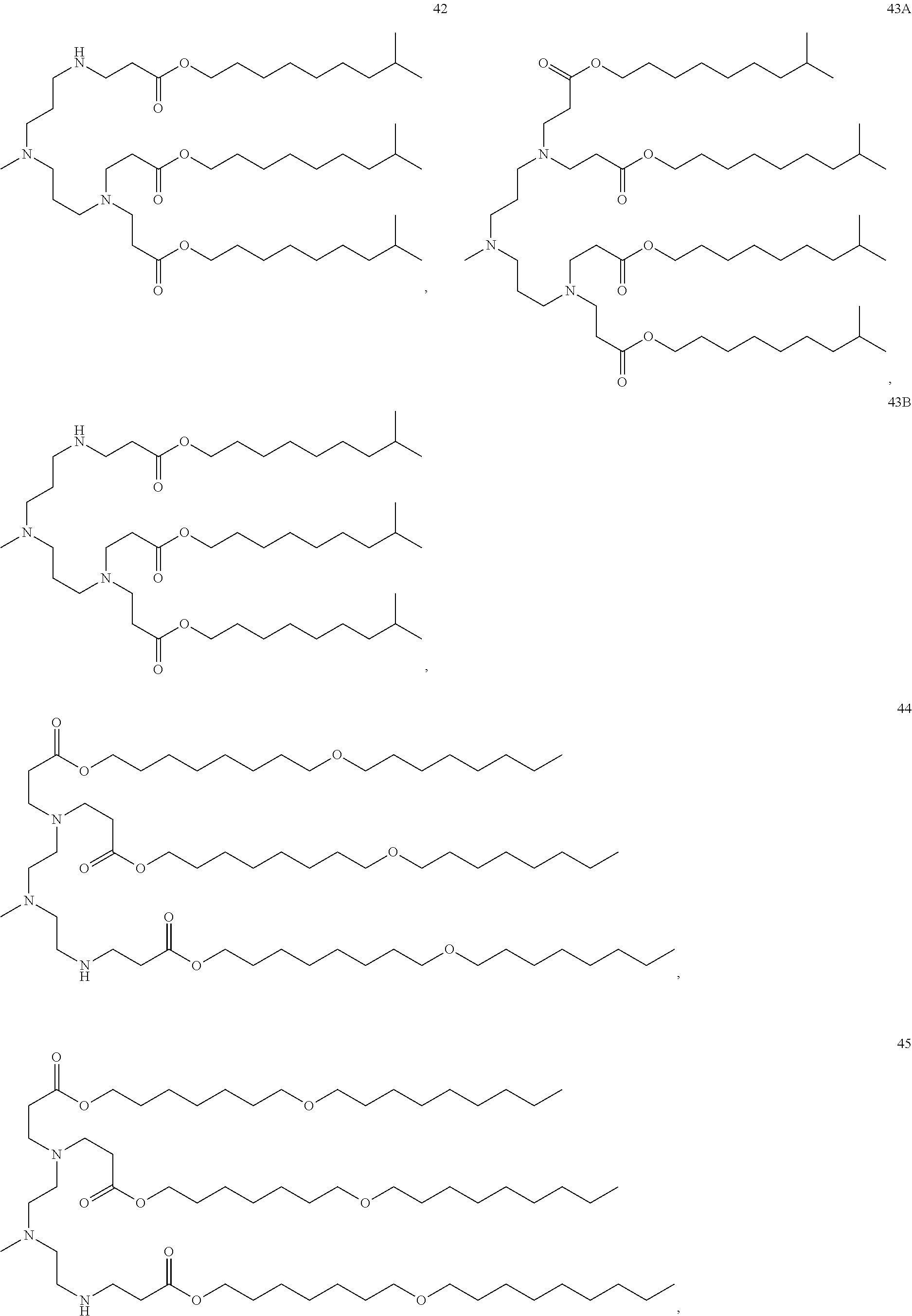 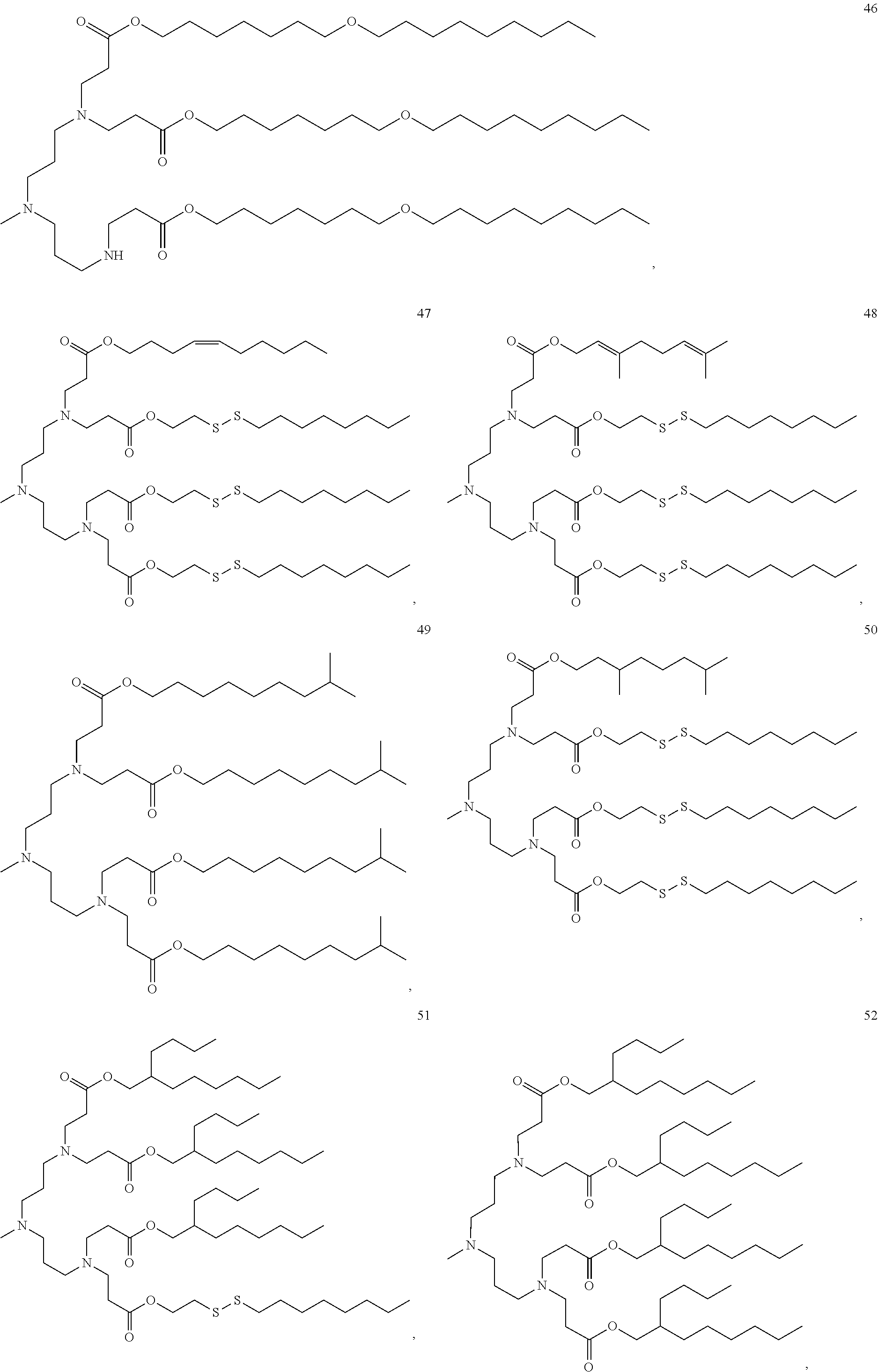 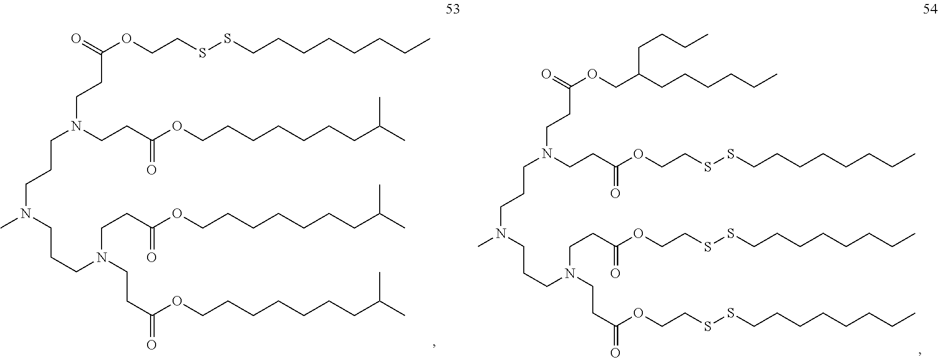 [SMILES: CCCCCCCCSSCCOC(=O)CCN(CCCN(C)CCCN(CCC(=O)OCCCCCCCC(C)C)CCC(=O)OCCCCCCCC(C)C)CCC(=O)OCCCCCCCC(C)C ; CCCCCCCCSSCCOC(=O)CCN(CCCN(C)CCCN(CCC(=O)OCCSSCCCCCCCC)CCC(=O)OCC(CCCC)CCCCCC)CCC(=O)OCCSSCCCCCCCC] 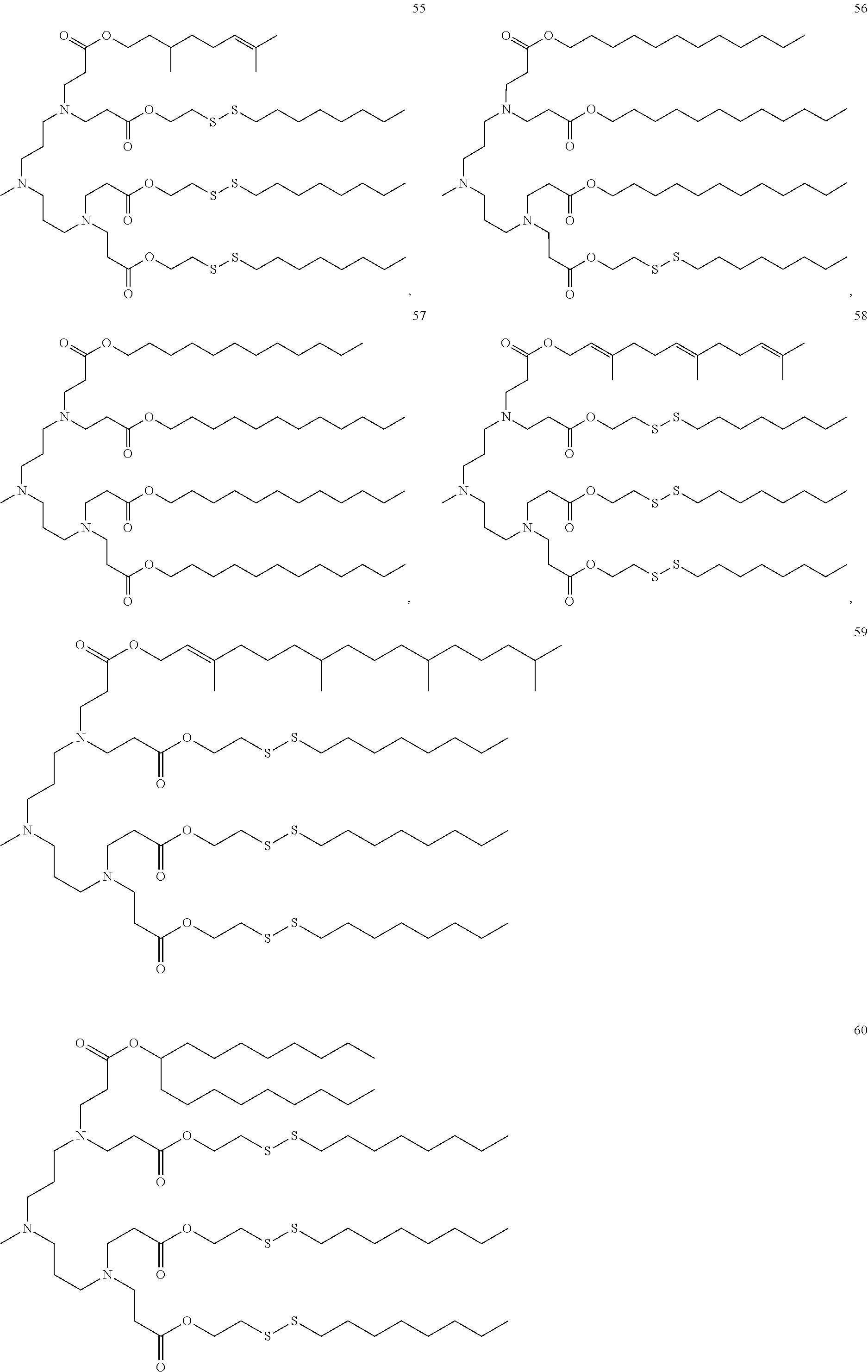 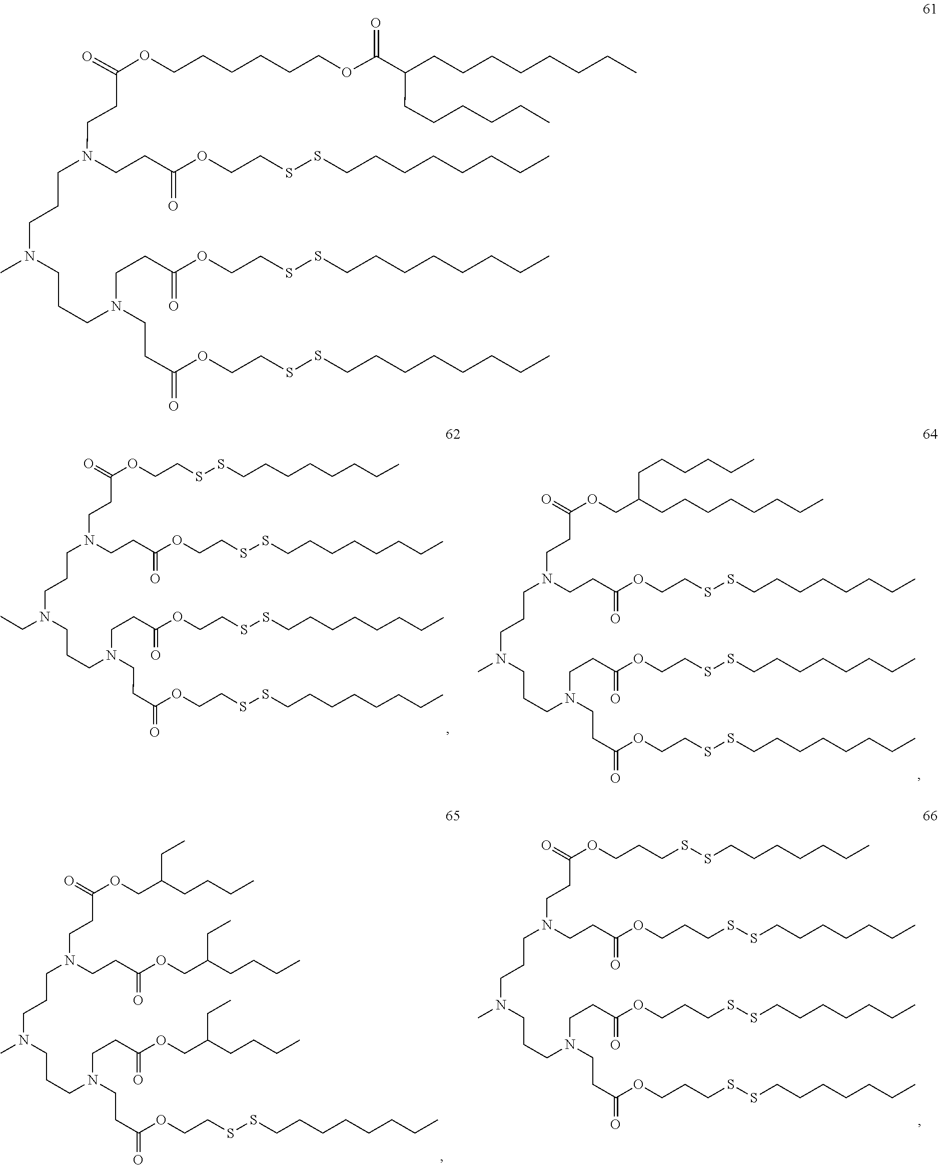 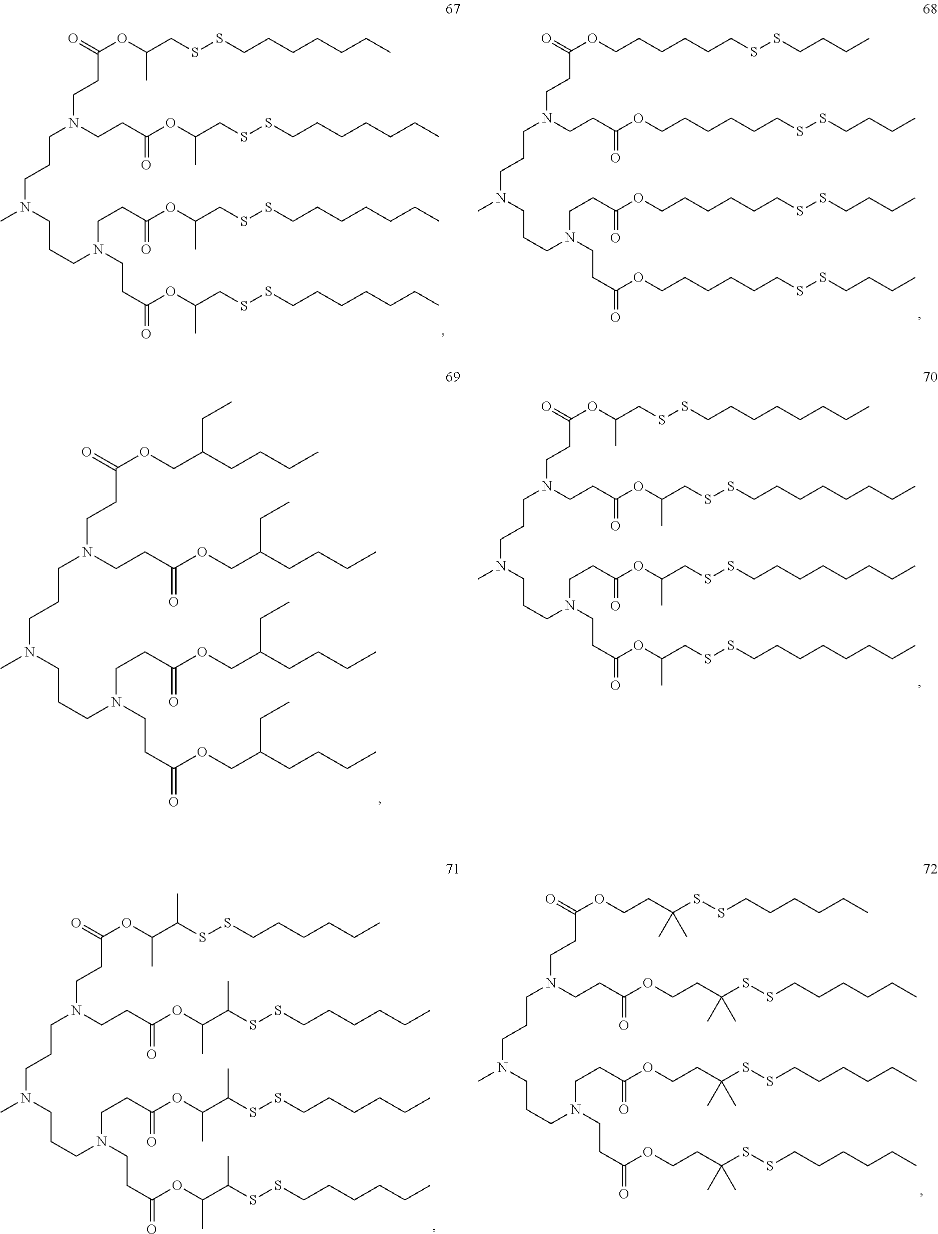 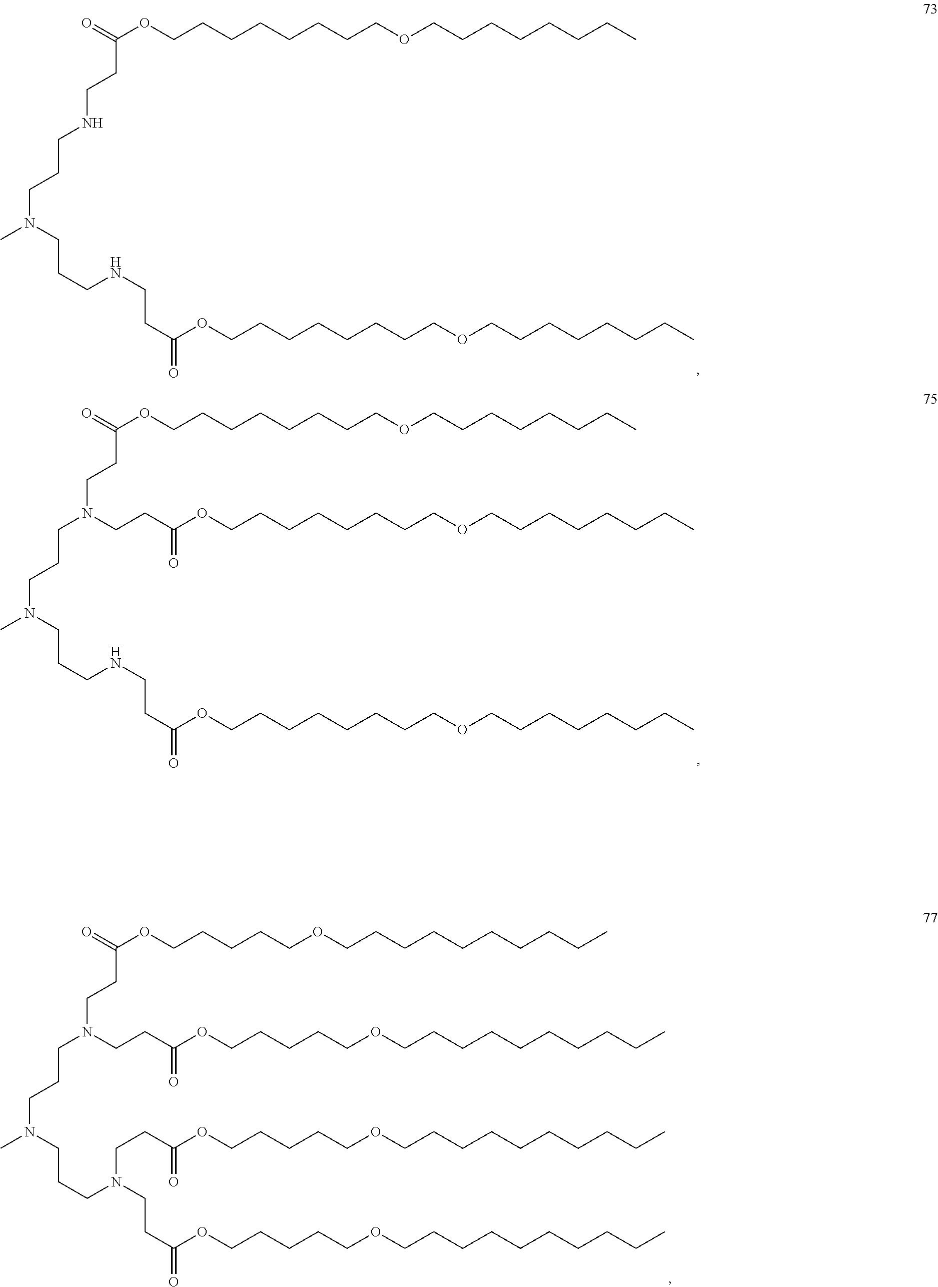 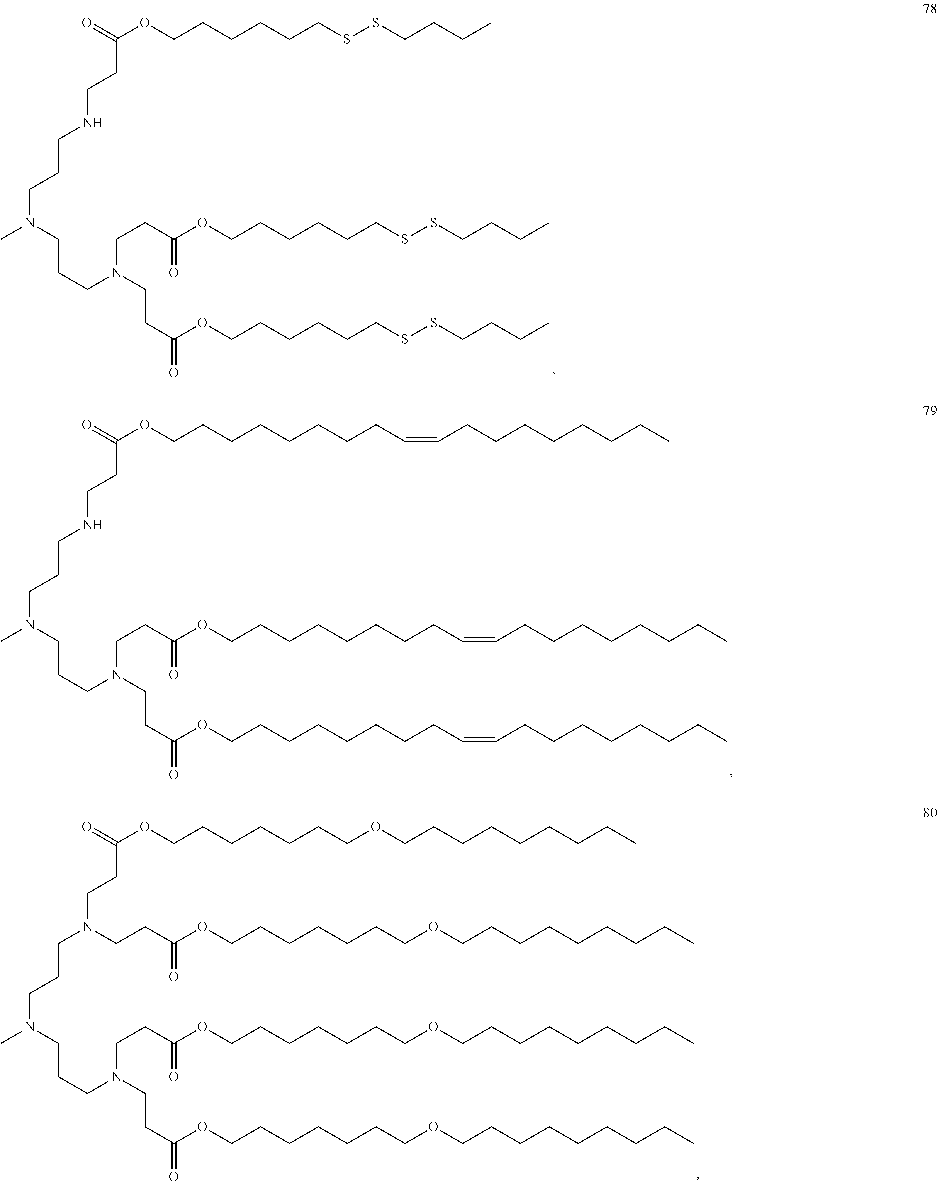 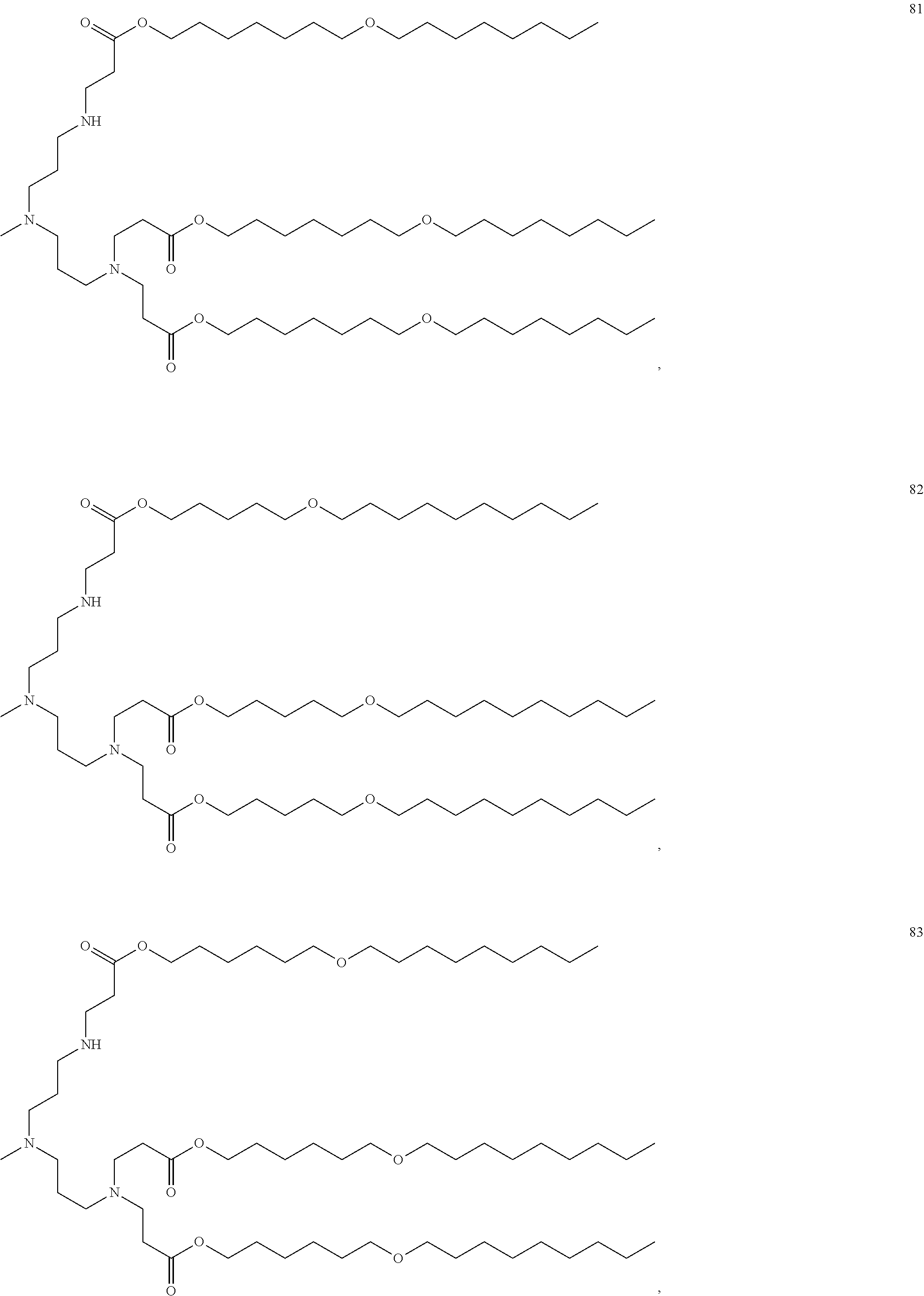 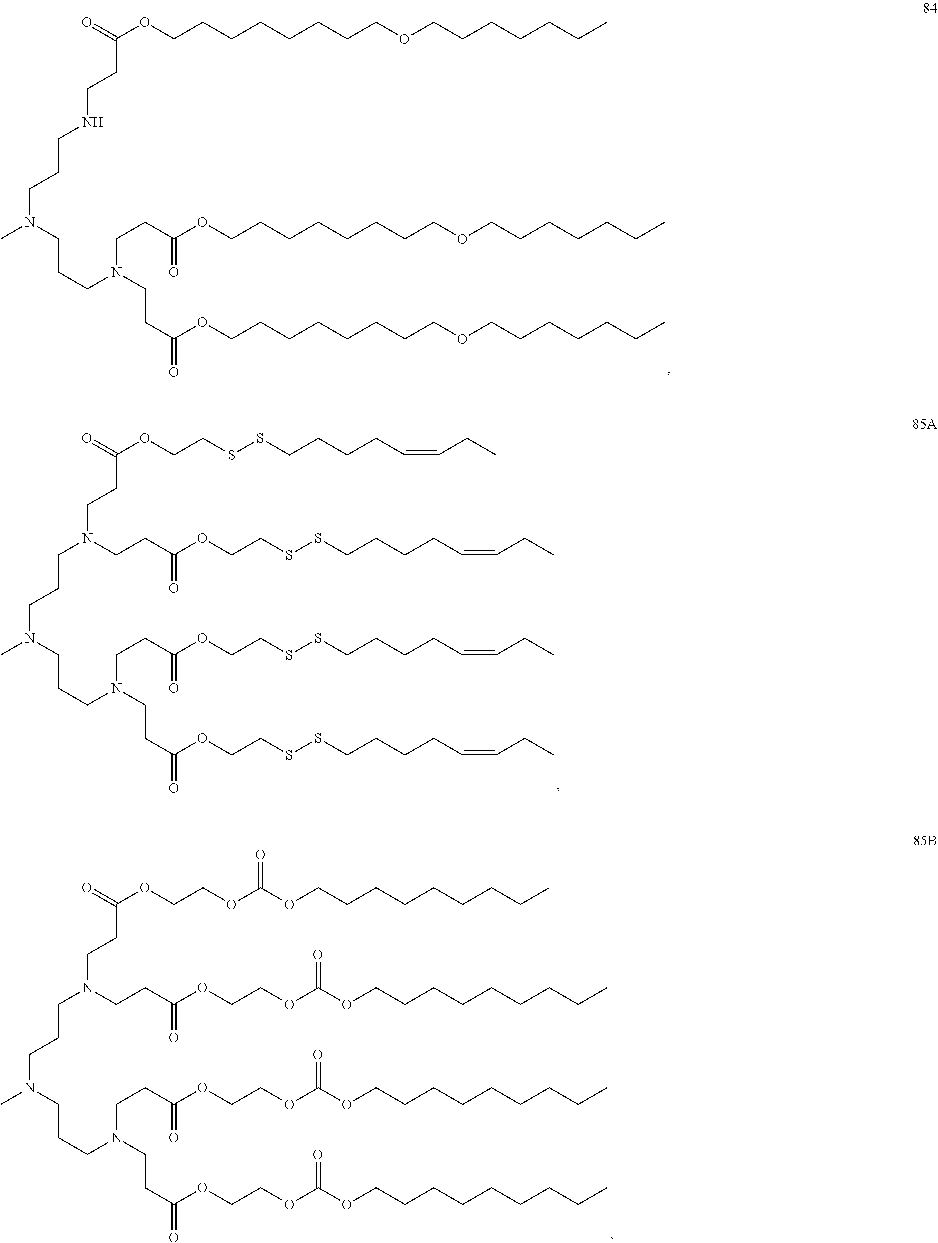 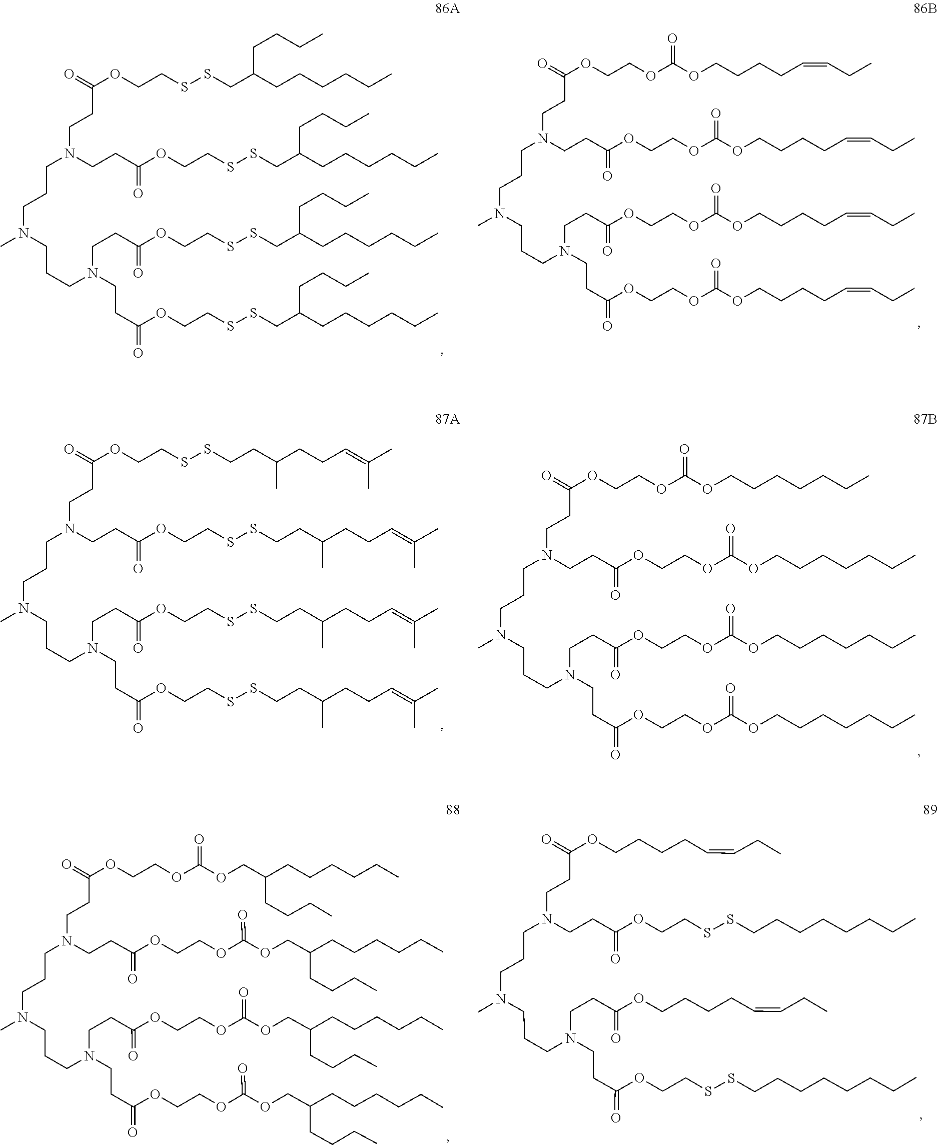 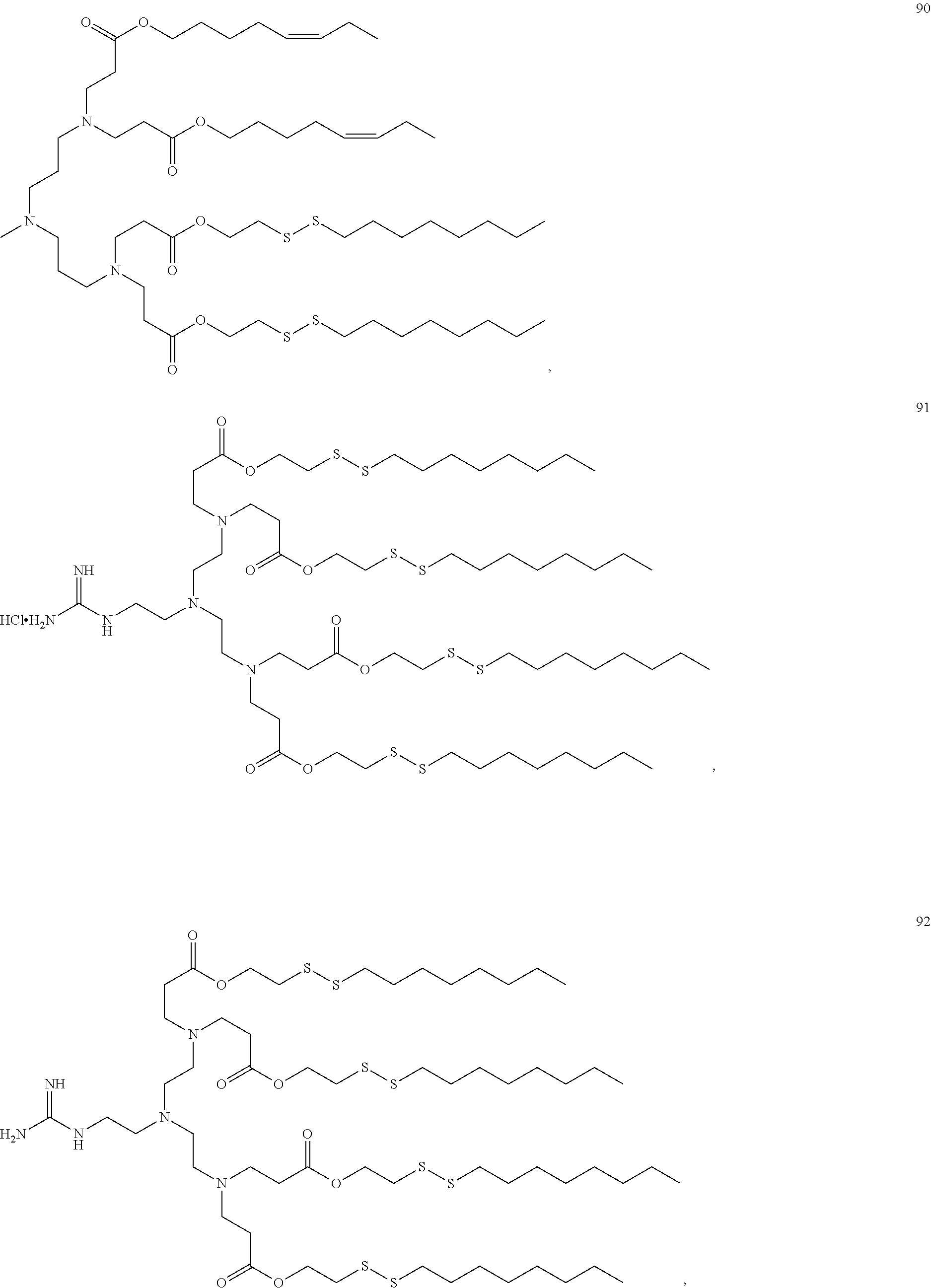 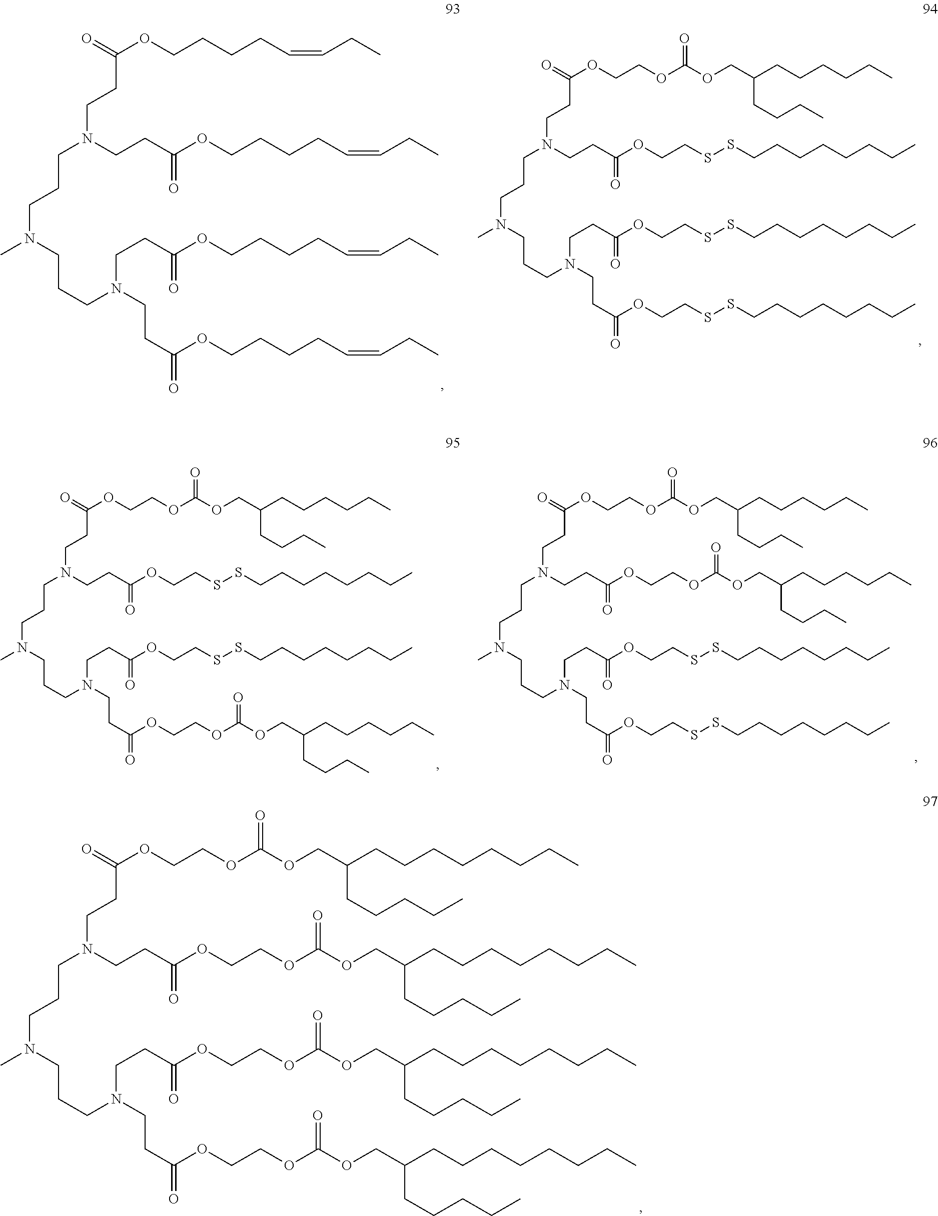 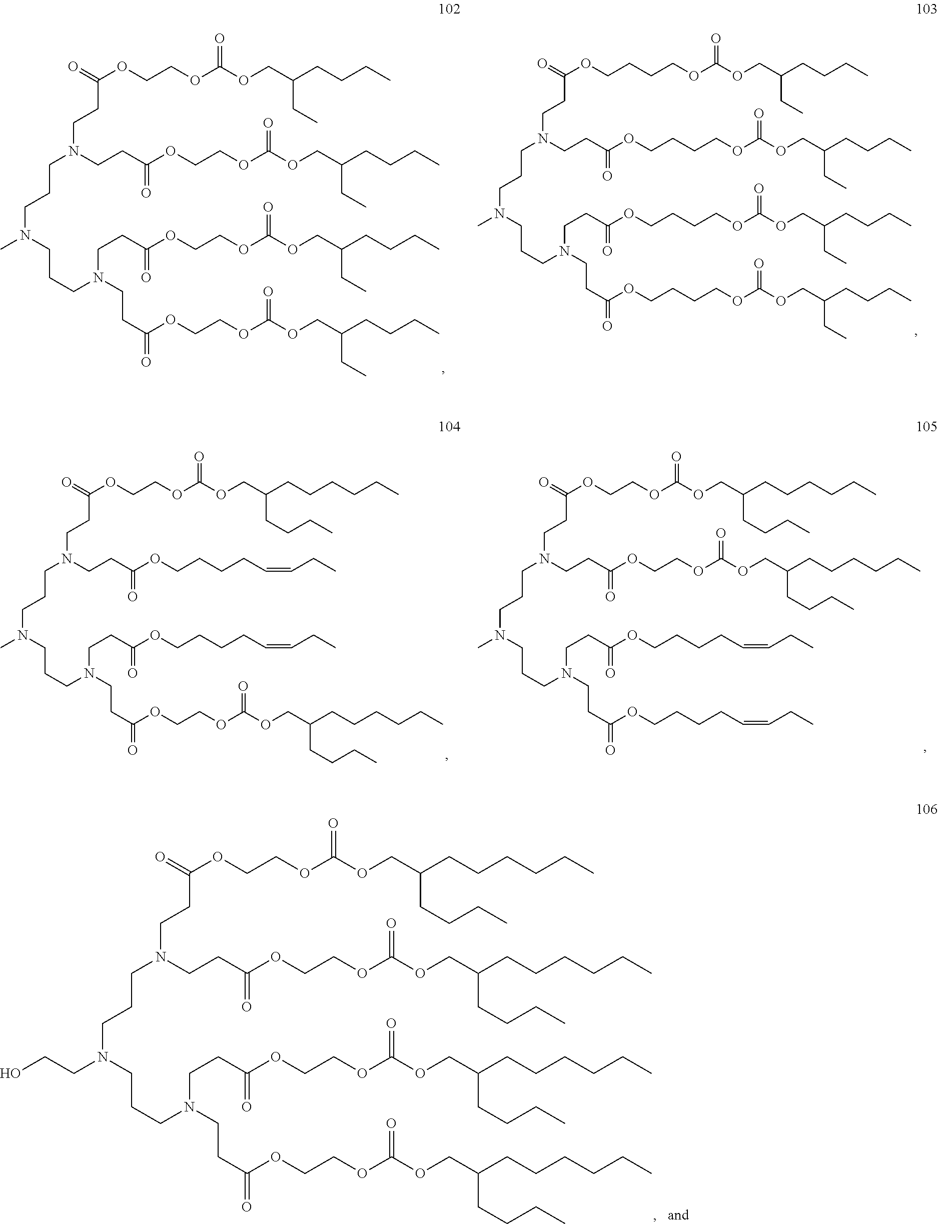 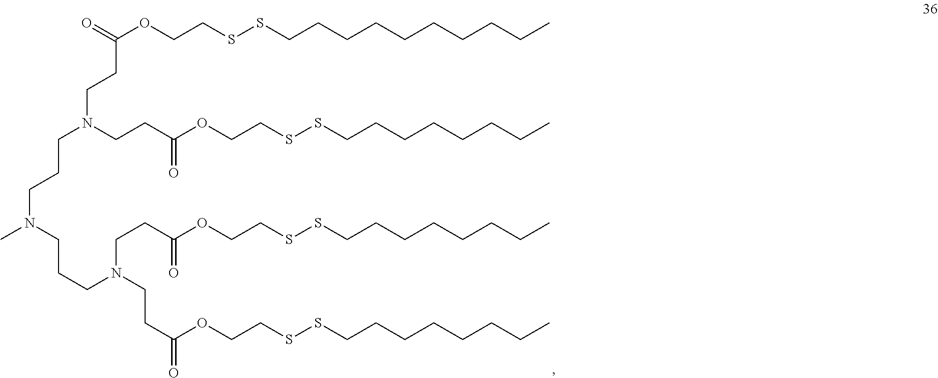 [SMILES: CCCCCCCCCCSSCCOC(=O)CCN(CCCN(C)CCCN(CCC(=O)OCCSSCCCCCCCC)CCC(=O)OCCSSCCCCCCCC)CCC(=O)OCCSSCCCCCCCC]
or a pharmaceutically acceptable salt thereof.

26. The composition of claim 1, wherein the lipidoid is: 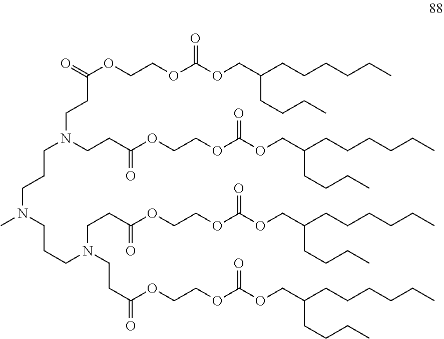 [SMILES: CCCCCCC(CCCC)COC(=O)OCCOC(=O)CCN(CCCN(C)CCCN(CCC(=O)OCCOC(=O)OCC(CCCC)CCCCCC)CCC(=O)OCCOC(=O)OCC(CCCC)CCCCCC)CCC(=O)OCCOC(=O)OCC(CCCC)CCCCCC]
or a pharmaceutically acceptable salt thereof.

27. A composition, comprising: a pharmaceutical agent and a lipid composition; wherein the pharmaceutical agent is assembled with the lipid composition; and the lipid composition comprises a lipidoid comprising an amine head group and at least one hydrophobic tail R^{Lipid}, wherein the amine head group has a structure of 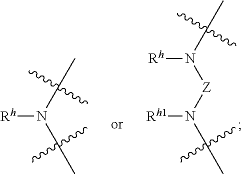
in which R^{h} and R^{h1} independently, is H, C₁₋₂₀ alkyl, C₂-C₂₀ alkenyl, C₂-C₂₀ alkynyl, C₃-C₂₀ cycloalkyl or heterocycloalkyl, C₁-C₂₀ heteroalkyl, C₃-C₂₀ aryl or heteroaryl, or a R^{Lipid}; and Z is a C₁-C₂₀ bivalent aliphatic radical, a C₁-C₂₀ bivalent heteroaliphatic radical, a bivalent aryl radical, or a bivalent heteroaryl radical;
wherein R^{Lipid} is selected from the group consisting of 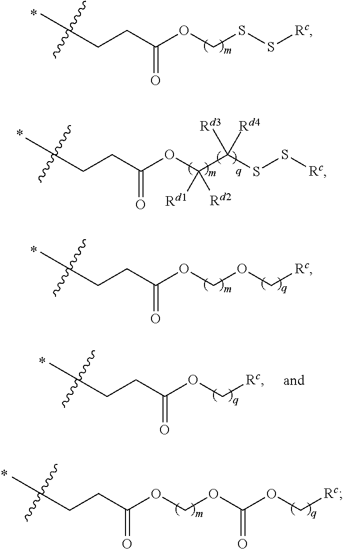
* indicates the point of attachment to N;
each R^{c} is independently an alkyl or an alkenyl;
R^{d1}, R^{d2}, R^{d3} and R^{d4} are each independently H or C₁-C₄ alkyl, wherein at least one of R^{d1}, R^{d2}, R^{d3} and R^{d4} is not H;
each m is independently an integer from 1-10; and
each q is independently an integer from 1-10.

28.-59. (canceled)

60. A method for preferential delivery of a pharmaceutical agent to liver or a liver cell in a subject in need thereof, the method comprising administering an effective amount of the composition according to claim 1, thereby providing a greater amount, expression or activity of the pharmaceutical agent in the liver or the liver cell of the subject as compared to that achieved in a non-liver organ or a non-liver cell in the subject.

61.-69. (canceled)

70. A method of vaccinating against a SARS-CoV2 variant comprising administering to a subject in need thereof an effective amount of the composition according to claim 1; wherein the composition comprises an mRNA encoding a spike protein of the SARS-CoV2.

71. A method of treating Cigler-Najjar (CN) syndrome comprising administering to a subject in need thereof an effective amount of the composition according to claim 1; wherein the composition comprises an mRNA encoding a UGTLAL protein.

72. A method of treating a dyslipidemia or a coronary artery disease comprising administering to a subject in need thereof an effective amount of the composition according to claim 1; wherein the composition comprises an mRNA encoding an ABE8e protein and a sgRNA targeting the ANPGTl3 gene.
